SEQUENCE LISTING (15 sequences)

SEQ ID NO: 1 - DNA, 756 bp, synthetic construct
  source 1..756: mol_type = other DNA
  misc_feature 1..756: note = Matrix protein 1
atgagcctcctgaccgaggtggagacctatgtgctgtccatcgtgcccagcggccctctgaaagccgagatcgcccagagactggaggatgtgttcgccggcaagaacaccgacctggaggctctgatggagtggctgaagaccaggcctatcctgtcccccctgaccaagggcatcctgggctttgtgttcaccctgaccgtgccctccgaaaggggactgcagaggaggaggttcgtgcagaatgccctgaacggcaacggcgaccctaacaacatggacagggccgtgaagctgtacaggaagctgaagagggagatcaccttccacggcgccaaggaggtggccctgagctacagcgctggagccctggccagctgcatgggcctgatttacaacaggatgggcaccgtgaccaccgaagtggccttcggcctggtgtgcgccacctgtgaacagatcgccgacagccagcataggagccatagacagatggtgaccaccacaaaccccctgatcaggcacgagaacagaatggtgctggcctccaccacagccaaggccatggagcagatggccggctccagcgaacaggctgccgaggccatggaggtggctagccaggccagacaaatggtccaagccatgaggaccatcggcacacaccctagcagcagcgctggcctgaaggacgatctgatcgagaacctgcaggcctaccagaagaggatgggcgtgcagatgcagaggttcaag

SEQ ID NO: 2 - DNA, 801 bp, synthetic construct
  source 1..801: mol_type = other DNA
  misc_feature 1..801: note = Matrix protein 1 with V5-tag
atgagcctcctgaccgaggtggagacctatgtgctgtccatcgtgcccagcggccctctgaaagccgagatcgcccagagactggaggatgtgttcgccggcaagaacaccgacctggaggctctgatggagtggctgaagaccaggcctatcctgtcccccctgaccaagggcatcctgggctttgtgttcaccctgaccgtgccctccgaaaggggactgcagaggaggaggttcgtgcagaatgccctgaacggcaacggcgaccctaacaacatggacagggccgtgaagctgtacaggaagctgaagagggagatcaccttccacggcgccaaggaggtggccctgagctacagcgctggagccctggccagctgcatgggcctgatttacaacaggatgggcaccgtgaccaccgaagtggccttcggcctggtgtgcgccacctgtgaacagatcgccgacagccagcataggagccatagacagatggtgaccaccacaaaccccctgatcaggcacgagaacagaatggtgctggcctccaccacagccaaggccatggagcagatggccggctccagcgaacaggctgccgaggccatggaggtggctagccaggccagacaaatggtccaagccatgaggaccatcggcacacaccctagcagcagcgctggcctgaaggacgatctgatcgagaacctgcaggcctaccagaagaggatgggcgtgcagatgcagaggttcaagggcaagcccattcctaaccctctgctgggcctggactccacatga

SEQ ID NO: 3 - DNA, 1536 bp, synthetic construct
  source 1..1536: mol_type = other DNA
  misc_feature 1..1536: note = Nucleoprote
atggcttcccagggcaccaagaggagctacgagcagatggagaccgacggagaaaggcagaacgccaccgaaatcagagccagcgtgggcaggatgatcggcggcatcggcaggttctacattcagatgtgcaccgagctgaagctgagcgactacgagggcagactgatccagaacagcatcaccatcgaaaggatggtcctgtccgctttcgacgagaggaggaacaagtacctggaagaacaccccagcgctggcaaggaccccaagaagaccggaggccccatctacaggagaatcgacggcaagtggatgagagagctcatcctctacgacaaggaggagatcagaaggatctggaggcaggctaataatggcgaagacgccaccgccggactgacccacatgatgatttggcacagcaatctgaacgacgctacctaccagaggaccagggccctcgtgaggacaggcatggaccccagaatgtgctccctgatgcaaggctccacactgcccaggaggagcggcgccgccggcgccgccgtcaaaggcgtgggaaccatggtgatggaactgattaggatgattaagaggggaatcaatgacaggaatttctggaggggagagaacggcagaaggaccagaatcgcctacgagaggatgtgcaacatcctcaagggcaagttccagaccgccgcccaaagggccatgatggaccaggtcagggagagcaggaatcctggcaacgccgaaatcgaggacctgattttcctggccaggagcgctctgatcctgaggggctccgtggcccacaagtcctgcctccctgcttgtgtgtacggccctgccgtggctagcggctacgacttcgagagggagggatatagcctggtcggaattgaccctttcagactgctgcagaacagccaggtctacagcctgatcaggcctaacgagaaccctgcccacaagagccagctggtgtggatggcctgccacagcgccgccttcgaggatctgagggtcagcagcttcatcagaggcacaagagtggtgcctagaggcaagctgtccacaaggggcgtgcagatcgccagcaacgagaatatggagaccatggattcctccaccctggaactgaggtccaggtactgggccatcagaaccaggtccggcggcaacacaaaccagcagagagccagcgctggccagatttccgtgcaacccacattctccgtgcagaggaatctgcccttcgagagggccaccatcatggccgctttcaccggaaacaccgagggaaggaccagcgatatgaggaccgagatcatcaggatgatggagtccgctaggcccgaggatgtgagcttccagggcaggggagtgttcgaactgtccgatgagaaagccacaagccctatcgtgccttccttcgacatgtccaacgagggctcctacttcttcggcgacaacgccgaggagtatgacaacgactataaggaccacgacggcgactacaaggatcacgacatc

SEQ ID NO: 4 - DNA, 1563 bp, synthetic construct
  source 1..1563: mol_type = other DNA; note = Nucleoprote in (NP) with Flag-tag
atggcttcccagggcaccaagaggagctacgagcagatggagaccgacggagaaaggcagaacgccaccgaaatcagagccagcgtgggcaggatgatcggcggcatcggcaggttctacattcagatgtgcaccgagctgaagctgagcgactacgagggcagactgatccagaacagcatcaccatcgaaaggatggtcctgtccgctttcgacgagaggaggaacaagtacctggaagaacaccccagcgctggcaaggaccccaagaagaccggaggccccatctacaggagaatcgacggcaagtggatgagagagctcatcctctacgacaaggaggagatcagaaggatctggaggcaggctaataatggcgaagacgccaccgccggactgacccacatgatgatttggcacagcaatctgaacgacgctacctaccagaggaccagggccctcgtgaggacaggcatggaccccagaatgtgctccctgatgcaaggctccacactgcccaggaggagcggcgccgccggcgccgccgtcaaaggcgtgggaaccatggtgatggaactgattaggatgattaagaggggaatcaatgacaggaatttctggaggggagagaacggcagaaggaccagaatcgcctacgagaggatgtgcaacatcctcaagggcaagttccagaccgccgcccaaagggccatgatggaccaggtcagggagagcaggaatcctggcaacgccgaaatcgaggacctgattttcctggccaggagcgctctgatcctgaggggctccgtggcccacaagtcctgcctccctgcttgtgtgtacggccctgccgtggctagcggctacgacttcgagagggagggatatagcctggtcggaattgaccctttcagactgctgcagaacagccaggtctacagcctgatcaggcctaacgagaaccctgcccacaagagccagctggtgtggatggcctgccacagcgccgccttcgaggatctgagggtcagcagcttcatcagaggcacaagagtggtgcctagaggcaagctgtccacaaggggcgtgcagatcgccagcaacgagaatatggagaccatggattcctccaccctggaactgaggtccaggtactgggccatcagaaccaggtccggcggcaacacaaaccagcagagagccagcgctggccagatttccgtgcaacccacattctccgtgcagaggaatctgcccttcgagagggccaccatcatggccgctttcaccggaaacaccgagggaaggaccagcgatatgaggaccgagatcatcaggatgatggagtccgctaggcccgaggatgtgagcttccagggcaggggagtgttcgaactgtccgatgagaaagccacaagccctatcgtgccttccttcgacatgtccaacgagggctcctacttcttcggcgacaacgccgaggagtatgacaacgactataaggaccacgacggcgactacaaggatcacgacatcgattataaggacgacgatgataagtga

SEQ ID NO: 5 - DNA, 2271 bp, synthetic construct
  source 1..2271: mol_type = other DNA
atggacgtcaaccccaccctgctcttcctgaaagtccctgctcagaacgccatctccaccacctttccctatacaggagaccccccctacagccacggaaccggaaccggatacaccatggacaccgtgaacagaacccatcagtactccgaaaaaggcaggtggaccaccaacaccgaaacaggagccccccagctgaatcctatcgatggccctctccccgaggataatgagccctccggctacgcccaaaccgactgtgtgctcgaggctatggctttcctggaggagagccaccctggcattttcgagaacagctgcctggagaccatggaggtggtgcagcagacaagagtcgataagctcacccaaggaaggcaaacctacgactggaccctcaataggaatcagcctgccgccaccgccctggccaatacaatcgaggtgttcaggagcaacggcctcaccgccaatgagtccggcagactgatcgacttcctgaaagacgtgatggagagcatggacaaagaagaaatggagatcaccacccattttcagaggaaaagaagggtgagggacaacatgacaaagaagatggtgacacagaggaccatcggcaagaaaaagcagaggctgaataaaagaagctatctgatcagggctctgaccctcaacacaatgaccaaagacgccgaaaggggaaaactcaagaggagggccattgccacccctggaatgcagatcaggggcttcgtctacttcgtcgagacactcgccaggtccatttgtgagaaactggagcagagcggcctgcctgtgggcggcaacgagaagaaggccaagctggccaacgtggtgaggaaaatgatgacaaatagccaggacacagagctgagcttcacaatcacaggcgataacaccaagtggaacgagaaccagaaccccaggatgttcctcgccatgattacctatattaccaggaaccagcctgagtggtttaggaacgtcctgagcatcgctcctatcatgttctccaataagatggccaggctgggaaaaggctatatgttcgagagcaagtccatgaaactgaggacccagatccccgccgagatgctggccagcatcgacctgaagtacttcaatgatagcaccaggaagaaaatcgaaaagatcagacccctgctcatcgatggcaccgcttccctgtcccccggaatgatgatgggcatgttcaatatgctgagcaccgtgctcggagtcagcattctcaacctcggccaaaaaaggtatacaaagacaacctactggtgggatggcctgcagagcagcgacgactttgccctgattgtgaacgcccccaatcacgagggcatccaggctggcgtcgacagattctacaggacctgcaaactgctgggcattaacatgtccaagaagaagagctacatcaacaggacaggcacattcgagttcacctccttcttctataggtacggctttgtggccaacttctccatggagctgccctccttcggcgtgtccggcattaatgagagcgccgacatgtccatcggcgtgacagtcatcaagaataacatgatcaataatgacctgggccctgccacagcccagatggccctgcagctgttcatcaaggattacagatacacatataggtgtcacaggggcgacacccagatccagaccagaaggtccttcgagatcaagaagctgtgggagcagacaagaagcaaggctggactcctggtgagcgacggcggccctaacctgtataacatcagaaatctgcatatccctgaggtgtgtctgaaatgggagctgatggacgaggattaccagggcagactctgtaaccccctcaacccctttgtgagccacaaggagatcgaatccgtgaacaacgccgtgatgatgcctgcccatggccctgccaagaatatggagtatgatgccgtcgccaccacccacagctggattcccaagaggaataggtccatcctgaatacaagccagagaggcatcctcgaagatgagcagatgtatcaaaaatgctgcaacctgttcgaaaagttcttcccctccagctcctataggaggcctgtgggaatcagcagcatggtggaggctatggtcagcagggccaggatcgatgctagaatcgacttcgagagcggcaggatcaagaaggaagagttcgccgagatcatgaagatttgcagcaccatcgaggagctgagaaggcagaag

SEQ ID NO: 6 - DNA, 2301 bp, synthetic construct
  source 1..2301: mol_type = other DNA; note = Polymerase B1 with HA-tag
atggacgtcaaccccaccctgctcttcctgaaagtccctgctcagaacgccatctccaccacctttccctatacaggagaccccccctacagccacggaaccggaaccggatacaccatggacaccgtgaacagaacccatcagtactccgaaaaaggcaggtggaccaccaacaccgaaacaggagccccccagctgaatcctatcgatggccctctccccgaggataatgagccctccggctacgcccaaaccgactgtgtgctcgaggctatggctttcctggaggagagccaccctggcattttcgagaacagctgcctggagaccatggaggtggtgcagcagacaagagtcgataagctcacccaaggaaggcaaacctacgactggaccctcaataggaatcagcctgccgccaccgccctggccaatacaatcgaggtgttcaggagcaacggcctcaccgccaatgagtccggcagactgatcgacttcctgaaagacgtgatggagagcatggacaaagaagaaatggagatcaccacccattttcagaggaaaagaagggtgagggacaacatgacaaagaagatggtgacacagaggaccatcggcaagaaaaagcagaggctgaataaaagaagctatctgatcagggctctgaccctcaacacaatgaccaaagacgccgaaaggggaaaactcaagaggagggccattgccacccctggaatgcagatcaggggcttcgtctacttcgtcgagacactcgccaggtccatttgtgagaaactggagcagagcggcctgcctgtgggcggcaacgagaagaaggccaagctggccaacgtggtgaggaaaatgatgacaaatagccaggacacagagctgagcttcacaatcacaggcgataacaccaagtggaacgagaaccagaaccccaggatgttcctcgccatgattacctatattaccaggaaccagcctgagtggtttaggaacgtcctgagcatcgctcctatcatgttctccaataagatggccaggctgggaaaaggctatatgttcgagagcaagtccatgaaactgaggacccagatccccgccgagatgctggccagcatcgacctgaagtacttcaatgatagcaccaggaagaaaatcgaaaagatcagacccctgctcatcgatggcaccgcttccctgtcccccggaatgatgatgggcatgttcaatatgctgagcaccgtgctcggagtcagcattctcaacctcggccaaaaaaggtatacaaagacaacctactggtgggatggcctgcagagcagcgacgactttgccctgattgtgaacgcccccaatcacgagggcatccaggctggcgtcgacagattctacaggacctgcaaactgctgggcattaacatgtccaagaagaagagctacatcaacaggacaggcacattcgagttcacctccttcttctataggtacggctttgtggccaacttctccatggagctgccctccttcggcgtgtccggcattaatgagagcgccgacatgtccatcggcgtgacagtcatcaagaataacatgatcaataatgacctgggccctgccacagcccagatggccctgcagctgttcatcaaggattacagatacacatataggtgtcacaggggcgacacccagatccagaccagaaggtccttcgagatcaagaagctgtgggagcagacaagaagcaaggctggactcctggtgagcgacggcggccctaacctgtataacatcagaaatctgcatatccctgaggtgtgtctgaaatgggagctgatggacgaggattaccagggcagactctgtaaccccctcaacccctttgtgagccacaaggagatcgaatccgtgaacaacgccgtgatgatgcctgcccatggccctgccaagaatatggagtatgatgccgtcgccaccacccacagctggattcccaagaggaataggtccatcctgaatacaagccagagaggcatcctcgaagatgagcagatgtatcaaaaatgctgcaacctgttcgaaaagttcttcccctccagctcctataggaggcctgtgggaatcagcagcatggtggaggctatggtcagcagggccaggatcgatgctagaatcgacttcgagagcggcaggatcaagaaggaagagttcgccgagatcatgaagatttgcagcaccatcgaggagctgagaaggcagaagtatccctacgatgtgcccgactatgcctga

SEQ ID NO: 7 - DNA, 982 bp, synthetic construct
  source 1..982: mol_type = other DNA; note = Influenza A virus matrix protein 1
atgagtcttttaaccgaggtcgaaacgtacgttctctctatcgtcccgtcaggccccctcaaagccgagatcgcgcagagacttgaagatgtctttgcagggaagaacaccgatcttgaggctctcatggaatggctaaagacaagaccaatcctgtcacctctgactaaggggattttaggatttgtgttcacgctcaccgtgcccagtgagcgaggactgcagcgtagacgctttgtccaaaatgcccttaatgggaacggggatccaaataacatggacagagcagttaaactgtacaggaagcttaagagggagataacattccatggggccaaagaagtagcactcagttattccgctggtgcacttgccagttgtatgggcctcatatacaacaggatggggactgtgaccactgaagtggcatttggcctggtatgcgcaacctgtgaacagattgctgattcccagcatcggtctcacaggcaaatggtgacaacaaccaatccactaatcagacatgagaacagaatggtactggccagcactacggctaaggctatggagcaaatggctggatcgagtgagcaagcagcagaggccatggaggttgctagtcaggctaggcaaatggtgcaggcgatgagaaccattgggactcatcctagctccagtgctggtctgaaagacgatcttattgaaaatttgcaggcctaccagaaacgaatgggggtgcagatgcaacgattcaagtgatcctctcgttattgccgcaagtatcattgggatcttgcacttgatattgtggattcttgatcgtctttttttcaaatgcatttatcgtcgccttaaatacggtttgaaaagagggccttctacggaaggagtgccggagtctatgagggaagaatatcgaaaggaacagcagagtgctgtggatgttgacgatggtcattttgtcaacatagagctggagtaa

SEQ ID NO: 8 - DNA, 1497 bp, synthetic construct
  source 1..1497: mol_type = other DNA
atggcgtctcaaggcaccaaacgatcttacgaacagatggagactgatggggaacgccagaatgccactgaaatcagagcatctgtcggaagaatgattggtggaattggacgattctacatccaaatgtgcaccgaacttaaactcagtgattatgagggacggctgatccagaacagcataacaatagagagaatggtgctctctgcttttgacgagaggaggaataaatatctggaagaacatcccagcgctgggaaagaccctaagaaaactggaggacccatatacaggagaatagatggaaagtggatgagagaactcatcctttatgacaaagaagaaataaggagaatctggcgccaagctaataatggtgaagatgcaacagctggtctgactcacatgatgatctggcattccaatttgaatgatgccacttatcagaggacaagagctcttgttcgcaccggaatggatcccaggatgtgctctctgatgcaaggttcaactctccctaggaggtctggagccgcaggtgctgcagtcaaaggagttggaacaatggtgatggagttgatcaggatgatcaaacgtgggatcaatgatcggaacttctggaggggtgagaatggacgaaggacaagaattgcctatgaaagaatgtgcaacattctcaaagggaaatttcaaacagctgcacagagagcaatgatggatcaagtgagagagagccggaatccaggaaatgctgaaattgaagatctcatctttctggcacggtctgcactcatattgagagggtcagttgctcacaagtcctgcctgcctgcctgtgtgtatggacctgctgtagccagtggatacgactttgaaagagagggatactctctggtcggaatagaccctttcagactgcttcaaaacagccaagtatacagtctaatcagaccaaatgagaatccagcacacaagagtcaactggtatggatggcatgccattctgctgcatttgaagatctgagagtatcaagcttcatcagagggacaagagtggttccaagagggaagctttccactagaggggttcagattgcttccaatgaaaacatggagactatggactccagtacccttgaactgagaagcagatactgggccataaggaccagaagtggaggaaacactaatcaacagagggcatctgcgggccaaatcagcgtgcaacctacattctcagtacagagaaacctcccttttgagagagcaaccattatggcagcattcactgggaacacagaggggagaacatctgacatgagaaccgaaatcataaggatgatggaaagtgcaagaccagaagatgtgtctttccaggggcggggagtcttcgagctctcggacgaaaaggcaacgagcccgatcgtgccttcctttgacatgagtaatgaaggatcttatttcttcggagacaatgcagaggagtacgacaattaa

SEQ ID NO: 9 - DNA, 2274 bp, synthetic construct
  source 1..2274: mol_type = other DNA; note = Influenza A virus polymerase PB1
atggatgtcaatccgactttacttttcttgaaagtgccagcgcaaaatgctataagcacaacgttcccttatactggagaccctccttacagccatgggacaggaacaggatacaccatggatactgtcaacaggacacatcagtactcagaaaagggaagatggacaacaaacaccgagactggagcaccacaactcaacccgattgatggaccactgccagaagacaatgaaccaagtggttatgcccaaacagattgtgtattggaagcaatggctttccttgaggagtcccaccccggtatctttgaaaactcgtgtcttgaaacgatggaggttgttcagcaaacacgagtggacaagctgacgcaaggccgacagacctatgactggactctaaataggaaccagcctgctgcaacagcattggccaacacaatagaagtgttcagatcaaatggcctcacggccaatgaatctggaaggctcatagacttcctcaaggatgtcatggagtcaatggacaaagaagaaatggaaatcacaactcattttcagagaaagagaagagtgagagacaacatgactaagaaaatggtgacacagagaacaataggtaaaaagaagcagaggttgaacaaaaggagttatctaatcagggcactgaccctgaacacaatgaccaaagatgctgagagagggaagctaaaacggagagcaattgcaaccccagggatgcaaatacgaggatttgtatactttgttgagacactggcaaggagtatatgtgagaaacttgaacaatcaggattgccagttggaggtaatgagaagaaagccaagttggcaaatgttgtaaggaagatgatgaccaattctcaggacactgagctttctttcaccatcactggagataacaccaaatggaacgaaaatcagaatcctcggatgtttttggccatgatcacatacataaccagaaatcagcccgaatggttcagaaatgttctaagtattgctccaataatgttctcaaacaaaatggcgagactggggaaggggtacatgttcgagagcaagagtatgaaacttagaactcaaatacctgcagagatgctagcaagcatcgatttgaaatatttcaatgattcaacaagaaagaaaattgaaaaaatccgaccgctcttaatagatgggactgcatcattgagccctggaatgatgatgggcatgttcaatatgttaagcactgtattaggcgtctccatcctgaatcttggacaaaagagatacaccaagactacttactggtgggatggtcttcaatcttctgatgattttgctctgattgtgaatgcacccaatcatgagggaattcaagccggagtcgacaggttttatcgaacctgtaagctgcttggaatcaatatgagcaagaaaaagtcttacataaacagaacaggcacatttgaattcacaagttttttctatcgctatgggtttgttgccaatttcagcatggagcttcccagctttggggtgtctgggatcaacgagtctgcggacatgagtattggagttactgtcatcaaaaacaatatgataaacaatgatcttggtccagcaacagctcaaatggcccttcagttgttcatcaaagattacaggtacacgtaccggtgccacaggggtgacacccaaatacaaacccgaagatcatttgaaataaagaaattgtgggagcaaacccgttccaaagctggactgctggtctccgacggaggccccaatttgtacaacattagaaatctccacattcctgaagtctgcttgaaatgggaattgatggatgaggattaccaggggcgtttatgcaacccactgaacccatttgtcagccataaagaaattgaatcagtgaataatgcagtgatgatgccagcacatggtccagccaaaaacatggagtatgatgctgttgcaacaacacactcctggattcccaaaagaaatcgatccatcttgaacacaagccagagaggaatacttgaagatgaacaaatgtaccaaaaatgctgcaatttatttgaaaaattcttccccagcagttcatacaggagaccagtcgggatttccagtatggtggaggccatggtttctagggcccgaattgatgcacggattgatttcgaatctggaaggataaagaaagaggagttcgctgagatcatgaagatctgttccaccattgaagagctcagacggcaaaagtag

SEQ ID NO: 10 - protein, 252 aa, synthetic construct
  source 1..252: note = Matrix protein
MSLLTEVETYVLSIVPSGPLKAEIAQRLEDVFAGKNTDLEALMEWLKTRPILSPLTKGILGFVFTLTVPSERGLQRRRFVQNALNGNGDPNNMDRAVKLYRKLKREITFHGAKEVALSYSAGALASCMGLIYNRMGTVTTEVAFGLVCATCEQIADSQHRSHRQMVTTTNPLIRHENRMVLASTTAKAMEQMAGSSEQAAEAMEVASQARQMVQAMRTIGTHPSSSAGLKDDLIENLQAYQKRMGVQMQRFK

SEQ ID NO: 11 - protein, 266 aa, synthetic construct
  source 1..266: note = Matrix protein with V5-tag
MSLLTEVETYVLSIVPSGPLKAEIAQRLEDVFAGKNTDLEALMEWLKTRPILSPLTKGILGFVFTLTVPSERGLQRRRFVQNALNGNGDPNNMDRAVKLYRKLKREITFHGAKEVALSYSAGALASCMGLIYNRMGTVTTEVAFGLVCATCEQIADSQHRSHRQMVTTTNPLIRHENRMVLASTTAKAMEQMAGSSEQAAEAMEVASQARQMVQAMRTIGTHPSSSAGLKDDLIENLQAYQKRMGVQMQRFKGKPIPNPLLGLDST

SEQ ID NO: 12 - protein, 512 aa, synthetic construct
  source 1..512: note = Nucleoprotein
MASQGTKRSYEQMETDGERQNATEIRASVGRMIGGIGRFYIQMCTELKLSDYEGRLIQNSITIERMVLSAFDERRNKYLEEHPSAGKDPKKTGGPIYRRIDGKWMRELILYDKEEIRRIWRQANNGEDATAGLTHMMIWHSNLNDATYQRTRALVRTGMDPRMCSLMQGSTLPRRSGAAGAAVKGVGTMVMELIRMIKRGINDRNFWRGENGRRTRIAYERMCNILKGKFQTAAQRAMMDQVRESRNPGNAEIEDLIFLARSALILRGSVAHKSCLPACVYGPAVASGYDFEREGYSLVGIDPFRLLQNSQVYSLIRPNENPAHKSQLVWMACHSAAFEDLRVSSFIRGTRVVPRGKLSTRGVQIASNENMETMDSSTLELRSRYWAIRTRSGGNTNQQRASAGQISVQPTFSVQRNLPFERATIMAAFTGNTEGRTSDMRTEIIRMMESARPEDVSFQGRGVFELSDEKATSPIVPSFDMSNEGSYFFGDNAEEYDNDYKDHDGDYKDHDI

SEQ ID NO: 13 - protein, 520 aa, synthetic construct
  source 1..520: note = Nucleoprotein with Flag-tag
MASQGTKRSYEQMETDGERQNATEIRASVGRMIGGIGRFYIQMCTELKLSDYEGRLIQNSITIERMVLSAFDERRNKYLEEHPSAGKDPKKTGGPIYRRIDGKWMRELILYDKEEIRRIWRQANNGEDATAGLTHMMIWHSNLNDATYQRTRALVRTGMDPRMCSLMQGSTLPRRSGAAGAAVKGVGTMVMELIRMIKRGINDRNFWRGENGRRTRIAYERMCNILKGKFQTAAQRAMMDQVRESRNPGNAEIEDLIFLARSALILRGSVAHKSCLPACVYGPAVASGYDFEREGYSLVGIDPFRLLQNSQVYSLIRPNENPAHKSQLVWMACHSAAFEDLRVSSFIRGTRVVPRGKLSTRGVQIASNENMETMDSSTLELRSRYWAIRTRSGGNTNQQRASAGQISVQPTFSVQRNLPFERATIMAAFTGNTEGRTSDMRTEIIRMMESARPEDVSFQGRGVFELSDEKATSPIVPSFDMSNEGSYFFGDNAEEYDNDYKDHDGDYKDHDIDYKDDDDK

SEQ ID NO: 14 - protein, 757 aa, synthetic construct
  source 1..757: note = Polymerase B1
MDVNPTLLFLKVPAQNAISTTFPYTGDPPYSHGTGTGYTMDTVNRTHQYSEKGRWTTNTETGAPQLNPIDGPLPEDNEPSGYAQTDCVLEAMAFLEESHPGIFENSCLETMEVVQQTRVDKLTQGRQTYDWTLNRNQPAATALANTIEVFRSNGLTANESGRLIDFLKDVMESMDKEEMEITTHFQRKRRVRDNMTKKMVTQRTIGKKKQRLNKRSYLIRALTLNTMTKDAERGKLKRRAIATPGMQIRGFVYFVETLARSICEKLEQSGLPVGGNEKKAKLANVVRKMMTNSQDTELSFTITGDNTKWNENQNPRMFLAMITYITRNQPEWFRNVLSIAPIMFSNKMARLGKGYMFESKSMKLRTQIPAEMLASIDLKYFNDSTRKKIEKIRPLLIDGTASLSPGMMMGMFNMLSTVLGVSILNLGQKRYTKTTYWWDGLQSSDDFALIVNAPNHEGIQAGVDRFYRTCKLLGINMSKKKSYINRTGTFEFTSFFYRYGFVANFSMELPSFGVSGINESADMSIGVTVIKNNMINNDLGPATAQMALQLFIKDYRYTYRCHRGDTQIQTRRSFEIKKLWEQTRSKAGLLVSDGGPNLYNIRNLHIPEVCLKWELMDEDYQGRLCNPLNPFVSHKEIESVNNAVMMPAHGPAKNMEYDAVATTHSWIPKRNRSILNTSQRGILEDEQMYQKCCNLFEKFFPSSSYRRPVGISSMVEAMVSRARIDARIDFESGRIKKEEFAEIMKICSTIEELRRQK

SEQ ID NO: 15 - protein, 766 aa, synthetic construct
MDVNPTLLFLKVPAQNAISTTFPYTGDPPYSHGTGTGYTMDTVNRTHQYSEKGRWTTNTETGAPQLNPIDGPLPEDNEPSGYAQTDCVLEAMAFLEESHPGIFENSCLETMEVVQQTRVDKLTQGRQTYDWTLNRNQPAATALANTIEVFRSNGLTANESGRLIDFLKDVMESMDKEEMEITTHFQRKRRVRDNMTKKMVTQRTIGKKKQRLNKRSYLIRALTLNTMTKDAERGKLKRRAIATPGMQIRGFVYFVETLARSICEKLEQSGLPVGGNEKKAKLANVVRKMMTNSQDTELSFTITGDNTKWNENQNPRMFLAMITYITRNQPEWFRNVLSIAPIMFSNKMARLGKGYMFESKSMKLRTQIPAEMLASIDLKYFNDSTRKKIEKIRPLLIDGTASLSPGMMMGMFNMLSTVLGVSILNLGQKRYTKTTYWWDGLQSSDDFALIVNAPNHEGIQAGVDRFYRTCKLLGINMSKKKSYINRTGTFEFTSFFYRYGFVANFSMELPSFGVSGINESADMSIGVTVIKNNMINNDLGPATAQMALQLFIKDYRYTYRCHRGDTQIQTRRSFEIKKLWEQTRSKAGLLVSDGGPNLYNIRNLHIPEVCLKWELMDEDYQGRLCNPLNPFVSHKEIESVNNAVMMPAHGPAKNMEYDAVATTHSWIPKRNRSILNTSQRGILEDEQMYQKCCNLFEKFFPSSSYRRPVGISSMVEAMVSRARIDARIDFESGRIKKEEFAEIMKICSTIEELRRQKYPYDVPDYA

(19) United States
(12) Patent Application Publication
(10) Pub. No.: US 2026/0091102 A1
(43) Pub. Date: Apr. 2, 2026

(54) UNIVERSAL T CELL-BASED, CMV-VECTORED VACCINE FOR INFLUENZA

(71) Applicant: Oregon Health & Science University, Portland, OR (US)

(72) Inventors: Jonah SACHA, Portland, OR (US); Louis PICKER, Portland, OR (US); Daniel MALOULI, Beaverton, OR (US); Scott HANSEN, Portland, OR (US); Klaus FRUEH, Portland, OR (US)

(73) Assignee: Oregon Health & Science University, Portland, OR (US)

(21) Appl. No.: 19/111,143

(22) PCT Filed: Sep. 13, 2023
(86) PCT No.: PCT/US2023/074072

§ 371 Date: Mar. 12, 2025

Related U.S. Application Data
(60) Provisional application No. 63/375,429, filed on Sep. 13, 2022.

(51) Int. Cl. A61K 39/145; A61K 39/00; A61K 40/11; A61K 40/32; A61P 31/16; C07K 14/725; C12N 5/0783; C12N 15/86; C12Q 1/6869

(52) U.S. Cl. CPC A61K 39/145; A61K 40/11; A61K 40/32; A61P 31/16; C07K 14/7051; C12N 5/0636; C12N 15/86; C12Q 1/6869; A61K 2039/5158; A61K 2039/525; A61K 2039/542; A61K 2039/57; C12N 2710/16143

(57) ABSTRACT

The invention relates to methods of generating an immune response for the treatment or prevention of a pathogenic infection. The invention also relates to methods of generating MHC-I, MHC-II, and/or MHC-E restricted CD8+ and/or CD4+ T cells for the treatment or prevention of a pathogenic infection.

### CROSS-REFERENCE TO RELATED APPLICATIONS

The present application claims the priority benefit of U.S. Provisional Application No. 63/375,429, filed Sep. 13, 2022, which is hereby incorporated by reference in its entirety.

### REFERENCE TO SEQUENCE LISTING SUBMITTED ELECTRONICALLY

The content of the electronically submitted sequence listing in ASCII text file (Name 4153_015PC01_SequenceListing_ST26; Size: 33,940 bytes; and Date of Creation: Aug. 18, 2023) filed with the application is incorporated herein by reference in its entirety.

### BACKGROUND OF THE INVENTION

The development of a universal influenza vaccine remains one of the top global health priorities, and novel approaches are urgently needed. An influenza pandemic has greater potential to cause large numbers of deaths and illnesses over a shorter time period than virtually any other natural health threat. Current antibody-eliciting influenza vaccines are extremely strain-specific due to the highly variable hemagglutinin (HA) and neuraminidase (NA) viral antigens they target. The best means of preventing or minimizing the global catastrophe that will result from widespread infection of a susceptible human population with a new influenza pandemic strain is prophylactic vaccination aimed at inducing broadly reactive, universal immune responses against a range of viral isolates and subtypes. For this reason, there is an urgent need to develop vaccines that control multiple strains over many years.

### BRIEF SUMMARY OF THE INVENTION

The present disclosure is directed to a recombinant cytomegalovirus (CMV) vector comprising: (a) a first nucleic acid sequence encoding an influenza matrix protein; (b) a second nucleic acid sequence encoding an influenza nucleoprotein; and (c) a third nucleic acid sequence encoding an influenza polymerase.

In some aspects, the vector does not express an active UL146 protein or ortholog thereof and does not express an active UL 147 protein or ortholog thereof.

The present disclosure is also directed to a recombinant cytomegalovirus (CMV) vector comprising: (a) a first nucleic acid sequence encoding an influenza matrix; and/or (b) a second nucleic acid sequence encoding an influenza nucleoprotein; and/or (c) a third nucleic acid sequence encoding an influenza polymerase; wherein the vector does not express an active UL128 protein or ortholog thereof; does not express an active UL130 protein or ortholog thereof; does not express an active UL 146 protein or ortholog thereof; does not express an active UL147 protein or ortholog thereof; and does not express an active UL 18 protein or ortholog thereof.

In one aspect, the CMV vector does not express an active UL18 protein or ortholog thereof. In another aspect, the CMV vector does not express an active pp71 protein or ortholog thereof.

In one aspect, the CMV vector is a human CMV (HCMV) vector, a rhesus macaque CMV (RhCMV) vector, or a cynomolgus macaque CMV (CyCMV) vector.

In one aspect, at least one of the influenza matrix protein, influenza nucleoprotein, or influenza polymerase is derived from an H1N1 influenza virus. In another aspect, the influenza matrix protein, influenza nucleoprotein, and influenza polymerase are derived from a H1N1 influenza virus. In another aspect, at least one of the influenza matrix protein, influenza nucleoprotein, or influenza polymerase is derived from a 1918 H1N1 influenza virus.

In one aspect, the influenza matrix protein, influenza nucleoprotein, and influenza polymerase are derived from a 1918 H1N1 influenza virus. In another aspect, the nucleic acid sequence encoding the matrix protein is a wild-type or a codon-optimized nucleic acid sequence. In another aspect, the nucleic acid sequence encoding the matrix protein is a codon-optimized nucleic acid sequence. In another aspect, the nucleic acid sequence encoding the matrix protein has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the sequence of SEQ ID NO: 1. In another aspect, the matrix protein has an amino acid sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the sequence of SEQ ID NO: 10.

In one aspect, the nucleic acid sequence encoding the nucleoprotein is a wild-type or a codon-optimized nucleic acid sequence. In another aspect, the nucleic acid sequence encoding the nucleoprotein is a codon-optimized nucleic acid sequence. In another aspect, the nucleic acid sequence encoding the nucleoprotein has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the sequence of SEQ ID NO: 3. In another aspect, the nucleoprotein has an amino acid sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the sequence of SEQ ID NO: 12.

In one aspect, the nucleic acid sequence encoding the polymerase encodes a polymerase basic 1 (PB1) protein, a polymerase basic 2 (PB2) protein, or a polymerase acidic protein. In another aspect, the nucleic acid sequence encoding the polymerase encodes a polymerase basic 1 (PB1) protein. In another aspect, the nucleic acid sequence encoding the polymerase is a wild-type or a codon-optimized nucleic acid sequence. In another aspect, the nucleic acid sequence encoding the polymerase is a codon-optimized nucleic acid sequence. In another aspect, the nucleic acid sequence encoding the polymerase has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the sequence of SEQ ID NO: 5. In another aspect, the polymerase has an amino acid sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the sequence of SEQ ID NO: 14.

In one aspect, the nucleic acid sequence encoding the matrix protein, the nucleic acid sequence encoding the nucleoprotein, and the nucleic acid sequence encoding the polymerase are under the transcriptional control of a UL82 promoter, CMV promoter, a CMV-gH promoter, a HCMV promoter, a RhCMV promoter, a EF1α promoter, a CAG promoter, or a MCMV promoter. In another aspect, the matrix, nucleoprotein, and polymerase are under the transcriptional control of a UL82 promoter.

The present disclosure is also directed to a pharmaceutical composition comprising a CMV vector described herein as a pharmaceutically acceptable carrier. The present disclosure is also directed to an immunogenic composition comprising a CMV vector described herein as a pharmaceutically acceptable carrier.

The present disclosure is also directed to a method of generating an immune response in a subject, comprising administering to the subject a CMV vector, pharmaceutical composition, or immunogenic composition disclosed herein in an amount effective to elicit a T cell response.

The present disclosure is also directed to the use of a CMV vector, pharmaceutical composition, or immunogenic composition disclosed herein in the manufacture of a medicament for use in generating an immune response in a subject.

The present disclosure is also directed to a method of generating an immune response in a subject to a pathogenic infection, comprising administering to the subject a CMV vector, pharmaceutical composition, or immunogenic composition disclosed herein in an amount effective to elicit a T cell response to a pathogen.

The present disclosure is also directed to the use of a CMV vector, pharmaceutical composition, or immunogenic composition disclosed herein in the manufacture of a medicament for use in generating an immune response to a pathogen in a subject.

The present disclosure is also directed to a method of treating or preventing a pathogenic infection in a subject, comprising administering a CMV vector, pharmaceutical composition, or immunogenic composition disclosed herein in an amount effective to elicit a T cell response to the pathogen.

The present disclosure is also directed to the use of a CMV vector, pharmaceutical composition, or immunogenic composition disclosed herein in the manufacture of a medicament for use in treating or preventing a pathogenic infection in a subject.

In one aspect, the method, CMV vector, pharmaceutical composition, or immunogenic composition for use, or use in manufacture disclosed herein, is an influenza virus. In another aspect, the influenza virus is a H1N1 virus, a H5N1 virus, a H2N2 virus, or a H3N2 virus. In another aspect, the pathogen is heterologous to the antigens encoded on the CMV vector. In another aspect, the pathogen is a H5N1 virus, a H2N2 virus, or a H3N2 virus. In another aspect, at least 10% of the CD8+ and/or CD4+ T cells elicited by the recombinant CMV vector are restricted by MHC-I or an ortholog thereof. In another aspect, at least 20%, at least 30%, at least 40%, at least 50%, at least 60%, at least 70%, at least 75%, at least 80%, at least 85%, at least 90%, or at least 95% of the CD8+ and/or CD4+ T cells elicited by the recombinant CMV vector are restricted by MHC-I or an ortholog thereof. In another aspect, at least 10% of the CD8+ and/or CD4+ T cells elicited by the recombinant CMV vector are restricted by MHC-E or an ortholog thereof. In another aspect, at least 20%, at least 30%, at least 40%, at least 50%, at least 60%, at least 70%, at least 75%, at least 80%, at least 85%, at least 90%, or at least 95% of the CD8+ and/or CD4+ T cells elicited by the recombinant CMV vector are restricted by MHC-E or an ortholog thereof. In another aspect, at least 10% of the CD8+ and/or CD4+ T cells elicited by the recombinant CMV vector are restricted by MHC-II or an ortholog thereof. In another aspect, at least 20%, at least 30%, at least 40%, at least 50%, at least 60%, at least 70%, at least 75%, at least 80%, at least 85%, at least 90%, or at least 95% of the CD8+ and/or CD4+ T cells elicited by the recombinant CMV vector are restricted by MHC-II or an ortholog thereof. In another aspect, fewer than 10%, fewer than 20%, fewer than 30%, fewer than 40%, or fewer than 50% of the CD8+ and/or CD4+ T cells elicited by the recombinant CMV vector are restricted by MHC-I or an ortholog thereof. In another aspect, fewer than 10% of the CD8+ and/or CD4+ T cells elicited by the recombinant CMV vector are restricted by MHC-I or an ortholog thereof. In another aspect, the subject is a human or non-human primate. In another aspect, administering the CMV vector comprises subcutaneous, intravenous, intramuscular, intraperitoneal, or oral administration of the CMV vector.

In one aspect, the methods further comprise identifying a T cell receptor (TCR) from the CD8+ and/or CD4+ T cells elicited by the CMV vector, wherein the TCR recognizes a MHC-E/influenza antigen-derived peptide complex. In another aspect, the method further comprises identifying a TCR from the CD8+ and/or CD4+ T cells elicited by the CMV vector, wherein the TCR recognizes a MHC-II/influenza antigen-derived peptide complex. In another aspect, the method further comprises identifying a TCR from the CD8+ and/or CD4+ T cells elicited by the CMV vector, wherein the TCR recognizes a MHC-I/influenza antigen-derived peptide complex. In another aspect, the TCR is identified by DNA or RNA sequencing.

The present disclosure is also directed to a method of generating CD8+ and/or CD4+ T cells that recognize MHC-E-peptide complexes, the method comprising: (1) administering to a subject a CMV vector disclosed herein in an amount effective to generate a set of CD8+ and/or CD4+ T cells that recognize MHC-E/peptide complexes; (2) identifying a first TCR from the set of CD8+ and/or CD4+ T cells, wherein the first TCR recognizes a MHC-E/influenza antigen-derived peptide complex; (3) isolating one or more CD8+ and/or CD4+ T cells from the subject; and (4) transfecting the one or more CD8+ and/or CD4+ cells with an expression vector, wherein the expression vector comprises a nucleic acid sequence encoding a second TCR and a promoter operably linked to the nucleic acid sequence encoding the second TCR, wherein the second TCR comprises CDR3α and CDR3β of the first TCR, thereby generating one or more transfected CD8+ and/or CD4+ T cells that recognize a MHC-E/influenza antigen-derived peptide complex.

In one aspect, the first TCR is identified by DNA or RNA sequencing. In another aspect, the second TCR comprises CDR1α, CDR2α, CDR3α, CDR1β, CDR2β, and CDR3β of the first TCR. In another aspect, the nucleic acid sequence encoding the second TCR is identical to the nucleic acid sequence encoding the first TCR.

In another aspect, administering the CMV vector to the subject comprises intravenous, intramuscular, intraperitoneal, or oral administration of the CMV vector to the subject. In another aspect, the subject is a human or nonhuman primate.

In another aspect, the method further comprises administering the transfected CD8+ and/or CD4+ T cells to the subject to treat a pathogenic infection. In another aspect, the pathogen is an influenza virus. In another aspect, the influenza virus is a H1N1 virus, a H5N1 virus, a H2N2 virus, or a H3N2 virus. In another aspect, the pathogen is heterologous to the antigens encoded on the CMV vector. In another aspect, the pathogen is a H5N1 virus, a H2N2 virus, or a H3N2 virus.

The present disclosure is also directed to a method of generating CD8+ and/or CD4+ T cells that recognize MHC-E-peptide complexes, the method comprising: (1) administering to a first subject a CMV vector disclosed herein in an amount effective to generate a set of CD8+ and/or CD4+ T cells that recognize MHC-E/peptide complexes; (2) identifying a first TCR from the set of CD8+ and/or CD4+ T cells, wherein the first TCR recognizes a MHC-E/influenza antigen-derived peptide complex; (3) isolating one or more CD8+ and/or CD4+ T cells from a second subject; and (4) transfecting the one or more CD8+ and/or CD4+ T cells with an expression vector, wherein the expression vector comprises a nucleic acid sequence encoding a second TCR and a promoter operably linked to the nucleic acid sequence encoding the second TCR, wherein the second TCR comprises CDR3α and CDR3β of the first TCR, thereby generating one or more transfected CD8+ and/or CD4+ T cells that recognize a MHC-E/influenza antigen-derived peptide complex.

In one aspect, the first TCR is identified by DNA or RNA sequencing. In another aspect, the first subject is a human or nonhuman primate. In another aspect, the second subject is a human or nonhuman primate. In another aspect, the first subject is a non-human primate and the second subject is a human and wherein the second TCR is a chimeric non-human primate-human TCR comprising the non-human primate CDR3α and CDR3β of the first TCR. In another aspect, the second TCR comprises the non-human primate CDR1α, CDR2α, CDR3α, CDR1β, CDR2β, and CDR3β of the first TCR. In another aspect, the nucleic acid sequence encoding the second TCR is identical to the nucleic acid sequence encoding the first TCR. In another aspect, the second TCR is a chimeric TCR. In another aspect, the nucleic acid sequence encoding the second TCR is identical to the nucleic acid sequence encoding the first TCR. In another aspect, administering the CMV vector to the subject comprises intravenous, intramuscular, intraperitoneal, or oral administration of the CMV vector to the subject.

In another aspect, the method further comprises administering the transfected CD8+ and/or CD4+ T cells to the subject to treat a pathogenic infection. In another aspect, the pathogen is an influenza virus. In another aspect, the influenza virus is a H1N1 virus, a H5N1 virus, a H2N2 virus, or a H3N2 virus. In another aspect, the pathogen is heterologous to the antigens encoded on the CMV vector. In another aspect, the pathogen is a H5N1 virus, a H2N2 virus, or a H3N2 virus.

The present disclosure is also directed to a method of generating CD8+ and/or CD4+ T cells that recognize MHC-II-peptide complexes, the method comprising: (1) administering to a subject a CMV vector disclosed herein in an amount effective to generate a set of CD8+ and/or CD4+ T cells that recognize MHC-II/peptide complexes; (2) identifying a first TCR from the set of CD8+ and/or CD4+ T cells, wherein the first TCR recognizes a MHC-II/influenza antigen-derived peptide complex; (3) isolating one or more CD8+ and/or CD4+ T cells from the subject; and (4) transfecting the one or more T cells with an expression vector, wherein the expression vector comprises a nucleic acid sequence encoding a second TCR and a promoter operably linked to the nucleic acid sequence encoding the second TCR, wherein the second TCR comprises CDR3α and CDR3β of the first TCR, thereby generating one or more transfected CD8+ and/or CD4+ T cells that recognize a MHC-II/influenza antigen-derived peptide complex. In one aspect, the first TCR is identified by DNA or RNA sequencing. In another aspect, the second TCR comprises CDR1α, CDR2α, CDR3α, CDR1B, CDR2β, and CDR3β of the first TCR. In another aspect, the nucleic acid sequence encoding the second TCR is identical to the nucleic acid sequence encoding the first TCR. In another aspect, administering the CMV vector to the subject comprises intravenous, intramuscular, intraperitoneal, or oral administration of the CMV vector to the subject. In another aspect, the subject is a human or nonhuman primate.

In one aspect, the method further comprises administering the transfected CD8+ and/or CD4+ T cells to the subject to treat a pathogenic infection. In another aspect, the pathogen is an influenza virus. In another aspect, the influenza virus is a H1N1 virus, a H5N1 virus, a H2N2 virus, or a H3N2 virus. In another aspect, the pathogen is heterologous to the antigens encoded by the CMV vector. In another aspect, the pathogen is a H5N1 virus, a H2N2 virus, or a H3N2 virus.

The present disclosure is also directed to a method of generating CD8+ and/or CD4+ T cells that recognize MHC-II-peptide complexes, the method comprising: (1) administering to a first subject a CMV vector disclosed herein in an amount effective to generate a set of CD8+ and/or CD4+ T cells that recognize MHC-II/peptide complexes; (2) identifying a first TCR from the set of CD8+ and/or CD4+ T cells, wherein the first TCR recognizes a MHC-II/influenza antigen-derived peptide complex; (3) isolating one or more T cells from a second subject; and (4) transfecting the one or more T cells with an expression vector, wherein the expression vector comprises a nucleic acid sequence encoding a second TCR and a promoter operably linked to the nucleic acid sequence encoding the second TCR, wherein the second TCR comprises CDR3α and CDR3β of the first TCR, thereby generating one or more transfected CD8+ and/or CD4+ T cells that recognize a MHC-II/influenza antigen-derived peptide complex. In one aspect, the first TCR is identified by DNA or RNA sequencing. In another aspect, the first subject is a human or nonhuman primate. In another aspect, the second subject is a human or nonhuman primate. In another aspect, the first subject is a non-human primate and the second subject is a human and wherein the second TCR is a chimeric non-human primate-human TCR comprising the non-human primate CDR3α and CDR3β of the first TCR. In another aspect, the second TCR comprises the non-human primate CDR1α, CDR2α, CDR3α, CDR1β, CDR2β, and CDR3β of the first TCR. In another aspect, the nucleic acid sequence encoding the second TCR is identical to the nucleic acid sequence encoding the first TCR. In another aspect, the second TCR is a chimeric TCR. In another aspect, the nucleic acid sequence encoding the second TCR is identical to the nucleic acid sequence encoding the first TCR. In another aspect, administering the CMV vector to the subject comprises intravenous, intramuscular, intraperitoneal, or oral administration of the CMV vector to the subject.

In one aspect, the method further comprises administering the transfected CD8+ and/or CD4+ T cells to the subject to treat a pathogenic infection. In another aspect, the pathogen is an influenza virus. In another aspect, the influenza virus a H1N1 virus, a H5N1 virus, a H2N2 virus, or a H3N2 virus. In another aspect, the pathogen is heterologous to the antigens encoded on the CMV vector. In another aspect, the pathogen a H5N1 virus, a H2N2 virus, or a H3N2 virus.

The present disclosure is also directed to a method of generating CD8+ and/or CD4+ T cells that recognize MHC-I-peptide complexes, the method comprising: (1) administering to a subject a CMV vector disclosed herein in an amount effective to generate a set of CD8+ and/or CD4+ T cells that recognize MHC-I/peptide complexes; (2) identifying a first TCR from the set of CD8+ and/or CD4+ T cells, wherein the first TCR recognizes a MHC-I/influenza antigen-derived peptide complex; (3) isolating one or more CD8+ and/or CD4+ T cells from the subject; and (4) transfecting the one or more CD8+ and/or CD4+ T cells with an expression vector, wherein the expression vector comprises a nucleic acid sequence encoding a second TCR and a promoter operably linked to the nucleic acid sequence encoding the second TCR, wherein the second TCR comprises CDR3α and CDR3β of the first TCR, thereby generating one or more transfected CD8+ and/or CD4+ T cells that recognize a MHC-I/influenza antigen-derived peptide complex.

In one aspect, the first TCR is identified by DNA or RNA sequencing. In another aspect, the second TCR comprises CDR1α, CDR2α, CDR3α, CDR1β, CDR2β, and CDR3β of the first TCR. In another aspect, the nucleic acid sequence encoding the second TCR is identical to the nucleic acid sequence encoding the first TCR. In another aspect, administering the CMV vector to the subject comprises intravenous, intramuscular, intraperitoneal, or oral administration of the CMV vector to the subject. In another aspect, the subject is a human or nonhuman primate.

In one aspect, the method further comprises administering the transfected CD8+ and/or CD4+ T cells to the subject to treat a pathogenic infection. In another aspect, the pathogen is an influenza virus. In another aspect, the influenza virus is a H1N1 virus, a H5N1 virus, a H2N2 virus, or a H3N2 virus. In another aspect, the pathogen is heterologous to the antigens encoded on the CMV vector. In another aspect, the pathogen is a H5N1 virus, a H2N2 virus, or a H3N2 virus.

The present disclosure is also directed to a method of generating CD8+ and/or CD4+ T cells that recognize MHC-I-peptide complexes, the method comprising: (1) administering to a first subject a CMV vector disclosed herein in an amount effective to generate a set of CD8+ and/or CD4+ T cells that recognize MHC-I/peptide complexes; (2) identifying a first TCR from the set of CD8+ and/or CD4+ T cells, wherein the first TCR recognizes a MHC-I/influenza antigen-derived peptide complex; (3) isolating one or more CD8+ and/or CD4+ T cells from a second subject; and (4) transfecting the one or more CD8+ and/or CD4+ T cells with an expression vector, wherein the expression vector comprises a nucleic acid sequence encoding a second TCR and a promoter operably linked to the nucleic acid sequence encoding the second TCR, wherein the second CDTCR comprises CDR3α and CDR3β of the first TCR, thereby generating one or more transfected CD8+ and/or CD4+ T cells that recognize a MHC-I/influenza antigen-derived peptide complex.

In one aspect, the first TCR is identified by DNA or RNA sequencing. In another aspect, the first subject is a human or nonhuman primate. In another aspect, the second subject is a human or nonhuman primate. In another aspect, the first subject is a non-human primate and the second subject is a human and wherein the second TCR is a chimeric non-human primate-human TCR comprising the non-human primate CDR3α and CDR3β of the first TCR. In another aspect, the second TCR comprises the non-human primate CDR1α, CDR2α, CDR3α, CDR1β, CDR2β, and CDR3β of the first TCR. In another aspect, the nucleic acid sequence encoding the second TCR is identical to the nucleic acid sequence encoding the first TCR. In another aspect, the second TCR is a chimeric TCR. In another aspect, the nucleic acid sequence encoding the second TCR is identical to the nucleic acid sequence encoding the first TCR. In another aspect, administering the CMV vector to the subject comprises intravenous, intramuscular, intraperitoneal, or oral administration of the CMV vector to the subject.

In one aspect, the method further comprises administering the transfected CD8+ and/or CD4+ T cells to the subject to treat a pathogenic infection. In another aspect, the pathogen is an influenza virus. In another aspect, the influenza virus is a H1N1 virus, a H5N1 virus, a H2N2 virus, or a H3N2 virus. In another aspect, the pathogen is heterologous to the antigens encoded on the CMV vector. In another aspect, the pathogen is a H5N1 virus, a H2N2 virus, or a H3N2 virus.

The present disclosure is also directed to a CD8+ and/or CD4+ T cell generated by a method disclosed herein.

The present disclosure is also directed to a method of treating or preventing a pathogenic infection in a subject, the method comprising administering a CD8+ and/or CD4+ T cell disclosed herein.

The present disclosure is also directed to the use of a CD8+ and/or CD4+ T cell disclosed herein in the manufacture of a medicament for use in treating or preventing a pathogenic infection in a subject.

The present disclosure is also directed to a recombinant cynomolgus cytomegalovirus (CyCMV) vector comprising at least one heterologous antigen, wherein the vector does not express an active UL128 protein or ortholog thereof; does not express an active UL130 protein or ortholog thereof; and does not express an active UL146 protein or ortholog thereof. In one aspect, the CyCMV vector does not express an active pp71 protein or ortholog thereof. In another aspect, the at least one heterologous antigen comprises a pathogen-specific antigen, a tumor antigen, a tissue-specific antigen, or a host self-antigen. In another aspect, the pathogen specific antigen is derived from a pathogen selected from the group consisting of human immunodeficiency virus, simian immunodeficiency virus, herpes simplex virus type 1, herpes simplex virus type 2, hepatitis B virus, hepatitis C virus, papillomavirus, Plasmodium parasites, and Mycobacterium tuberculosis. In another aspect, the tumor antigen is related to a cancer selected from the group consisting of acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, breast cancer, lung cancer, ovarian cancer, prostate cancer, pancreatic cancer, colon cancer, renal cell carcinoma (RCC), and germ cell tumors.

The present disclosure is also directed to a pharmaceutical composition comprising a CyCMV vector disclosed herein and a pharmaceutically acceptable carrier.

The present disclosure is also directed to an immunogenic composition comprising a CyCMV vector disclosed herein and a pharmaceutically acceptable carrier.

The present disclosure is also directed to a method of generating an immune response in a subject, comprising administering to the subject a CyCMV vector disclosed herein in an amount effective to elicit a CD8+ and/or CD4+ T cell response.

The present disclosure is also directed to the use of a CyCMV vector disclosed herein in the manufacture of a medicament for use in generating an immune response in a subject.

The present disclosure is also directed to a method of generating an immune response in a subject to a pathogenic infection, comprising administering to the subject a CyCMV vector disclosed herein in an amount effective to elicit a CD8+ and/or CD4+ T cell response to a pathogen.

The present disclosure is also directed to the use of a CyCMV vector disclosed herein in the manufacture of a medicament for use in generating an immune response to a pathogen in a subject.

The present disclosure is also directed to a method of treating or preventing a pathogenic infection in a subject, comprising administering a CyCMV vector disclosed herein in an amount effective to elicit a CD8+ and/or CD4+ T cell response to the pathogen.

The present disclosure is also directed to the use of a CyCMV vector disclosed herein in the manufacture of a medicament for use in treating or preventing a pathogenic infection in a subject.

In one aspect of the CyCMV vector, the pathogen is a pathogen selected from the group consisting of: human immunodeficiency virus, simian immunodeficiency virus, herpes simplex virus type 1, herpes simplex virus type 2, hepatitis B virus, hepatitis C virus, papillomavirus, Plasmodium parasites, and Mycobacterium tuberculosis.

The present disclosure is also directed to a method of treating a tumor in a subject, comprising administering a CyCMV vector disclosed herein in an amount effective to elicit a CD8+ and/or CD4+ T cell response to the tumor.

The present disclosure is also directed to the use of a CyCMV vector disclosed herein in the manufacture of a medicament for use in treating a tumor in a subject.

In one aspect, the tumor is related to a cancer selected from the group consisting of: acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, breast cancer, lung cancer, ovarian cancer, prostate cancer, pancreatic cancer, colon cancer, renal cell carcinoma (RCC), and germ cell tumors.

In one aspect, the method, CMV vector, pharmaceutical composition, or immunogenic composition for use, or use in manufacture is directed to methods wherein at least 10% of the CD8+ and/or CD4+ T cells elicited by the recombinant CMV vector are restricted by MHC-E or an ortholog thereof. In another aspect, wherein at least 20%, at least 30%, at least 40%, at least 50%, at least 60%, at least 70%, at least 75%, at least 80%, at least 85%, at least 90%, or at least 95% of the CD8+ and/or CD4+ T cells elicited by the recombinant CMV vector are restricted by MHC-E or an ortholog thereof. In another aspect, at least 10% of the CD8+ and/or CD4+ T cells elicited by the recombinant CMV vector are restricted by MHC-II or an ortholog thereof. In another aspect, at least 20%, at least 30%, at least 40%, at least 50%, at least 60%, at least 70%, at least 75%, at least 80%, at least 85%, at least 90%, or at least 95% of the CD8+ and/or CD4+ T cells elicited by the recombinant CMV vector are restricted by MHC-II or an ortholog thereof. In another aspect, fewer than 10%, fewer than 20%, fewer than 30%, fewer than 40%, or fewer than 50% of the CD8+ and/or CD4+ T cells elicited by the recombinant CMV vector are restricted by MHC-I or an ortholog thereof. In another aspect, fewer than 10% of the CD8+ and/or CD4+ T cells elicited by the recombinant CMV vector are restricted by MHC-I or an ortholog thereof. In another aspect, the subject is a human or non-human primate. In another aspect, administering the CMV vector comprises subcutaneous, intravenous, intramuscular, intraperitoneal, or oral administration of the CMV vector.

In one aspect, the methods further comprise identifying a TCR from the CD8+ and/or CD4+ T cells elicited by the CMV vector, wherein the TCR recognizes a MHC-E/heterologous antigen-derived peptide complex. In another aspect, the method further comprises identifying a TCR from the CD8+ and/or CD4+ T cells elicited by the CMV vector, wherein the TCR recognizes a MHC-II/heterologous antigen-derived peptide complex. In another aspect, the TCR is identified by DNA or RNA sequencing.

### BRIEF DESCRIPTION OF THE DRAWINGS/FIGURES

FIG. 1 shows the epitope targeting and MHC restriction of SIVmac239gag specific CD8+ T cells elicited by a CyCMV ΔCy157.5/ΔCy157.4 vaccine vector. Individual peptides resulting in specific CD8+ T cell responses are indicated by a box, with the color of the box indicating MHC restriction, as determined by blocking with the anti-pan-MHC-1 mAB W6/32 (which blocks recognition of both the non-polymorphic MHC-E and polymorphic MHC-Ia molecules), the MHC-E blocking peptide VL9, and the MHC-II blocking antibody G46.6.

FIG. 2 shows the epitope targeting and MHC restriction of SIVmac239gag specific CD8+ T cells elicited by a CyCMV ΔCy 157.5/ΔCy 157.4 ΔCy 158-Cy161 (double deleted) vaccine vector expressing the SIV Gag antigen. Individual peptides resulting in specific CD8+ T cell responses are indicated by a box, with the color of the box indicating MHC restriction, as determined by blocking with the anti-pan-MHC-1 mAB W6/32 (which blocks recognition of both the non-polymorphic MHC-E and polymorphic MHC-Ia), the MHC-E blocking peptide VL9, and the MHC-II blocking antibody G46.6.

FIGS. 3A-3C show the generation of dissemination impaired full-length (FL) CyCmv and double deleted (dd) CyCmv vectorslacking the homologs of HCMV UL128, UL130 and UL146. FIG. 3A is a schematic showing FL CyCMV vectors: FL CyCmv (first row), FL CyCMV ΔCy 110 in which the Cy110 gene encoding for pp71 was replaced with a gene encoding the 1918 influenza matrix protein tagged with a V5 epitope tag (second row), FL CyCMV ΔCy110 (pp71) expressing the 1918 influenza nucleoprotein tagged with the FLAG epitope by replacing Cy 110 (third row), and FL CyCMV ΔCy110 (pp71) expressing the 1918 influenza polymerase basic 1 (PB-1) protein tagged with HA-epitopes by replacing Cy110 (fourth row). FIG. 3B is a schematic showing dd CyCmv vectors deleted for two gene regions Cy 157.5-Cy157.4 (homologs of HCMV UL 128 and UL130) and Cy 158-Cy161, homologs of HCMV UL146. dd CyCmv (first row), dd CyCMV ΔCy110 (pp71) expressing the 1918 influenza matrix protein with a V5 tag by replacing Cy110 (second row), dd CyCMV ΔCy110 (pp71) expressing the 1918 influenza nucleoprotein with a FLAG tag by replacing Cy110 (third row), and dd CyCMV ΔCy110 (pp71) expressing the 1918 influenza polymerase basic 1 (PB-1) protein with a HA tag by replacing Cy 110 (fourth row). FIG. 3C is an immunoblot showing the protein expression of V5-tagged 1918 influenza matrix protein (MI), FLAG-tagged 1918 influenza nucleoprotein (NP), or HA-tagged 1918 influenza polymerase basic 1 (PB1) from uninfected cells or from cells infected with FL CyCMV ΔCy110 (pp71)+1918 influenza matrix protein with a V5 tag, FL CyCMV ΔCy110 (pp71)+1918 influenza nucleoprotein with a FLAG tag, FL CyCMV ΔCy110 (pp71)+1918 influenza polymerase basic 1 (PB-1) protein with a HA tag, dd CyCMV ΔCy110 (pp71)+1918 influenza matrix protein with a V5 tag, dd CyCMV ΔCy110 (pp71)+1918 influenza nucleoprotein with a Flag tag, or dd CyCMV ΔCy110 (pp71)+1918 influenza polymerase basic 1 (PB-1) protein with a HA tag.

FIGS. 4A-4F show the immunology of MCM vaccinated with dissemination impaired full length (FL) CyCMVΔCy110+1918 influenza matrix (M), nucleoprotein (NP), or polymerase basic 1 (PB-1) proteins (FL CyCMV ΔCy110/flu) or double deleted CyCMV+1918 influenza matrix (M), nucleoprotein (NP), or polymerase basic 1 (PB-1) proteins (dd CyCMVΔCy110/flu). FIG. 4A is a schematic depicting the MCM vaccination and challenge schedule. FIG. 4B are graphs showing the induction of influenza-specific CD4+ (left) or CD8+ (right) T cells in peripheral blood mononuclear cells (PBMC) of MCM vaccinated with either FL CyCMVΔCy 110/flu or dd CyCMVΔCy 110/flu up to 161 days post vaccination. FIG. 4C are graphs showing the induction of influenza-specific CD4+ T cells in peripheral blood mononuclear cells (PBMC) (top left), CD8+ T cells in PBMCs (top right), CD4+ T cells in bronchoalveolar lavage (BAL) (bottom left), and CD8+ T cells in bronchoalveolar lavage (bottom right) of MCM vaccinated with either FL CyCMVΔCy 110/flu or dd CyCMVΔCy110/flu. Responses are shown as frequencies of memory T cells in individual animals responding to all influenza antigens (total, left panels) or individual matrix protein (M), nucleoprotein (NP), and polymerase basic-1 protein (PB1) antigens (right panels). Open circles denote MCM that succumbed to challenge while closed circles denote those that survived. FIG. 4D shows the epitope targeting and MHC restriction of influenza specific CD8+ T cells elicited by the dissemination-impaired dd CyCMVΔCy 110/flu vaccine vector (left) or a FL CyCMVΔCy 110/flu vaccine vector (right). Individual peptides resulting in specific CD8+ T cell responses are indicated by a box, with the color of the box indicating MHC restriction, as determined by blocking with the anti-pan-MHC-1 mAB W6/32 (which blocks recognition of both the non-polymorphic MHC-E and polymorphic MHC-Ia molecules), the MHC-E blocking peptide VL9, and the MHC-II blocking antibody. FIG. 4E are graphs showing the memory phenotype of influenza nucleoprotein (NP) specific CD4+ T cells (left) or CD8+ T cells (right) as determined by CCR7 and CD28 expression in PBMC of MCM vaccinated with either FL CyCMVΔCy 110/flu or dd CyCMVΔCy110/flu. TCM-central memory T cells; TTHEM-resident memory T cells; TEM-effector memory T cells. Open circles denote MCM that succumbed to challenge while closed circles denote those that survived. FIG. 4F are graphs showing the recognition of the various inactivated whole influenza isolates (H5N1, H5N2, H7N7, H9N2, H1N1, H7N9, H5N6, and H3N2) indicated on the x axis by CD4+ (left) or CD8+ (right) T cells in the PBMC of dd CyCMVΔCy110/Flu-vaccinated (black) and FL CyCMVΔCy110/Flu-vaccinated (red) MCM following overnight co-culture. Note that open circles denote MCM that succumbed to challenge while closed circles denote those that survived. FIG. 4G is a graph showing the polyfunctionality profile of CD4+ or CD8+ T cells targeting the nucleoprotein insert

FIGS. 5A-5E show the response of dissemination impaired CyCMVΔCy110/influenza-vaccinated or unvaccinated MCM to challenge with aerosolized H5N1. FIG. 5A is a graph showing the viral titer of the aerosolized H5N1 inoculum used to challenge unvaccinated MCM (blue), dd CyCMVΔCy110/flu vaccinated MCM (black), or FL CyCMVΔCy110/flu vaccinated MCM (red). Open circles denote MCM that succumbed to challenge. NS-not significant. FIG. 5B is a graph showing the H5N1 viral titer in bronchoalveolar lavage (BAL) of unvaccinated MCM (blue), FL CyCMVΔCy110/flu vaccinated MCM (red), and dd CyCMVΔCy110/flu vaccinated MCM (black) after challenge with H5N1. FIG. 5C is a graph showing the thoracic radiographic scores of unvaccinated MCM (blue), FL CyCMVΔCy 110/flu vaccinated MCM (red), and dd CyCMVΔCy110/flu-vaccinated MCM (black) after challenge with H5N1. Open circles denote MCM that succumbed to the infection. FIG. 5D are graphs showing the temperature change from baseline in unvaccinated MCM (blue, left), dd CyCMV/flu vaccinated MCM (black, middle), and FL CyCMVΔCy110/flu vaccinated MCM (red, right) after challenge with H5N1. Open circles denote MCM that succumbed to the infection. FIG. 5E is a survival curve showing the survival of unvaccinated MCM (blue), FL CyCMVΔCy 110/flu vaccinated MCM (red), and dd CyCMVΔCy110/flu vaccinated MCM (black) after challenge with H5N1. FIG. 5F is a graph showing the respiratory frequency in unvaccinated MCM (blue), FL CyCMV/influenza-vaccinated MCM (red), and dd CyCMV/influenza-vaccinated MCM (black) after challenge with H5N1. FIG. 5G is a graph showing the inspiratory time of unvaccinated MCM (blue), FL CyCMV/influenza-vaccinated MCM (red), and dd CyCMV/influenza-vaccinated MCM (black) after challenge with H5N1.

FIGS. 6A-6B compare the immune responses in CyCMVΔCy 110/flu vaccinated MCM that survived or succumbed to lethal H5N1 influenza challenge. FIG. 6A are graphs showing the frequencies of influenza-specific CD4+ T cells in peripheral blood mononuclear cells (PBMC) (top left), CD8+ T cells in PBMCs (top right), CD4+ T cells in bronchoalveolar lavage (BAL) (bottom left), and CD8+ T cells in bronchoalveolar lavage (bottom right) in terminal or surviving MCM vaccinated with either FL CyCMVΔCy110/flu or dd CyCMVΔCy110/flu. T cell response frequencies are shown against all influenza antigens (total, left panels) and individual matrix protein (M), nucleoprotein (NP), and polymerase basic-1 protein (PB1) antigens (right panels). FIG. 6B is a time series heatmap of the expression of IL-15 protection signature genes in dd CyCMV/SIV vaccinated RhCMV (left) compared to dd CyCMVΔCy110/flu (middle) and FL CyCMVΔCy110/flu (right) vaccinated MCM. Protected animals are denoted with a red heading bar versus unprotected animals in black. Plotted are log 2 fold change values of the 122 leading edge genes across the vaccination time series (Days 0, 1, 3, and 7 post vaccination). dd CyCMV/SIV data are from Malouli et al. Cell Host & Microbe 2022.

### DETAILED DESCRIPTION OF THE INVENTION

### I. Terms

Unless otherwise noted, technical terms are used according to conventional usage.

All publications, patents, patent applications, internet sites, and accession numbers/database sequences (including both polynucleotide and polypeptide sequences) cited herein are hereby incorporated by reference in their entirety for all purposes to the same extent as if each individual publication, patent, patent application, internet site, or accession number/database sequence were specifically and individually indicated to be so incorporated by reference.

Although methods and materials similar or equivalent to those described herein may be used in the practice or testing of this disclosure, suitable methods and materials are described below. In addition, the materials, methods, and examples are illustrative only and not intended to be limiting. In order to facilitate review of the various embodiments of the disclosure, the following explanations of specific terms are provided.

Antigen: As used herein, the terms “antigen” or “immunogen” are used interchangeably to refer to a substance, typically a protein, which is capable of inducing an immune response in a subject. The term also refers to proteins that are immunologically active in the sense that once administered to a subject (either directly or by administering to the subject a nucleotide sequence or vector that encodes the protein) the protein is able to evoke an immune response of the humoral and/or cellular type directed against that protein.

Antigen-specific T cell: A CD8⁺ or CD4⁺ lymphocyte that recognizes a particular antigen. Generally, antigen-specific T cells specifically bind to a particular antigen presented by MHC molecules, but not other antigens presented by the same MHC. In some aspects, the antigen-specific T cell binds to an influenza antigen.

Administration: As used herein, the term “administration” means to provide or give a subject an agent, such as a composition comprising an effective amount of a CMV vector comprising an exogenous antigen by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes.

Codon optimized: As used herein, the term “codon optimized” refers to the coding region or gene of a nucleic acid molecule for transformation of various hosts, so as to reflect the typical codon usage of the host organism without altering the polypeptide encoded by the DNA. Refers to altering the codon in the coding region or gene of the nucleic acid molecule. In the context of the present invention, gene and DNA coding regions are codon-optimized for optimal expression in mammalian cells. The codon-optimization can synthesize all of the DNA (or a portion of it) to remove all the destabilizing sequences or regions of the secondary structure that may be present in the transcribed mRNA, it may synthesize all of the DNA (or some portion), or it may change the DNA to be more preferred in the desired host cell.

Effective amount: As used herein, the term “effective amount” refers to an amount of an agent, such as a CMV vector comprising a heterologous antigen (e.g., an influenza antigen) or a transfected CD8+ and/or CD4+ T cell that recognizes a MHC-E/heterologous antigen (e.g., an influenza antigen)-derived peptide complex, a MHC-II/heterologous antigen (e.g., an influenza antigen)-derived peptide complex, or a MHC-I/heterologous antigen (e.g., an influenza antigen)-derived peptide complex, that is sufficient to generate a desired response, such as reduce or eliminate a sign or symptom of a condition or disease or induce an immune response to an antigen. In some examples, an “effective amount” is one that treats (including prophylaxis) one or more symptoms and/or underlying causes of any of a disorder or disease. An effective amount may be a therapeutically effective amount, including an amount that prevents one or more signs or symptoms of a particular disease or condition from developing, such as one or more signs or symptoms associated with infectious disease or cancer.

Heterologous antigen: As used herein, the term “heterologous antigen” refers to any protein or fragment thereof that is not derived from CMV. Heterologous antigens may be pathogen-specific antigens, tumor virus antigens, tumor antigens, host self-antigens, or any other antigen. In some aspects, heterologous antigens are influenza antigens.

Immune response: As used herein, the term “immune response” refers to the concerted action of lymphocytes, antigen presenting cells, phagocytic cells, granulocytes, and soluble macromolecules produced by the above cells or the liver (including antibodies, cytokines, chemokines, interferons, and complement) that results in selective damage to, destruction of, or elimination from the human body of cancerous cells, metastatic tumor cells, malignant melanoma, invading pathogens, cells or tissues infected with pathogens, or, in cases of autoimmunity or pathological inflammation, normal human cells or tissues.

Influenza: As used herein, the term “influenza” refers to a contagious respiratory illness or infection caused by a number of viruses. Symptoms of the flu include extreme tiredness, muscle aches, chills, cough, fever, headache, sore throat, runny or stuffy nose, nausea, vomiting, and diarrhea. Influenza viruses are divided into three types, designated A, B, and C. Influenza types A and B are responsible for epidemics of respiratory illness that occur almost every winter and are often associated with increased rates of hospitalization and death. Type C infection usually causes either a very mild respiratory illness or no symptoms at all; it does not cause epidemics and does not have the severe public health impact of influenza types A and B.

Mutation: As used herein, the term “mutation” refers to any difference in a nucleic acid or polypeptide sequence from a normal, consensus, or “wild type” sequence. A mutant is any protein or nucleic acid sequence comprising a mutation. In addition, a cell or an organism with a mutation may also be referred to as a mutant. Some types of coding sequence mutations include point mutations (differences in individual nucleotides potentially resulting in amino acid changes or in amino acids themselves); silent mutations (differences in nucleotides that do not result in an amino acid changes); deletions (differences in which one or more nucleotides are missing, up to and including a deletion of the entire coding sequence of a gene); frameshift mutations (differences in which deletion of a number of nucleotides indivisible by 3 results in an alteration of the amino acid sequence). A mutation that results in a difference in an amino acid may also be called an amino acid substitution mutation. Amino acid substitution mutations may be described by the amino acid change relative to wild type at a particular position in the amino acid sequence.

Nucleotide sequences or nucleic acid sequences: The terms “nucleotide sequences” and “nucleic acid sequences” refer to deoxyribonucleic acid (DNA) or ribonucleic acid (RNA) sequences, including, without limitation, messenger RNA (mRNA), DNA/RNA hybrids, or synthetic nucleic acids. The nucleic acid may be single-stranded, or partially or completely double stranded (duplex). Duplex nucleic acids may be homoduplex or heteroduplex.

Operably Linked: As the term “operably linked” is used herein, a first nucleic acid sequence is operably linked with a second nucleic acid sequence when the first nucleic acid sequence is placed in such a way that it has an effect upon the second nucleic acid sequence. Operably linked DNA sequences may be contiguous, or they may operate at a distance.

Promoter: As used herein, the term “promoter” may refer to any of a number of nucleic acid control sequences that directs transcription of a nucleic acid, for example mRNA. Typically, a eukaryotic promoter includes necessary nucleic acid sequences near the start site of transcription, such as, in the case of a polymerase II type promoter, a TATA element or any other specific DNA sequence that is recognized by one or more transcription factors. Expression by a promoter may be further modulated by enhancer or repressor elements. Numerous examples of promoters are available and well known to those of ordinary skill in the art. A nucleic acid comprising a promoter operably linked to a nucleic acid sequence that codes for a particular polypeptide may be termed an expression vector.

Recombinant: As used herein, the term “recombinant” with reference to a nucleic acid or polypeptide refers to one that has a sequence that is not naturally occurring or has a sequence that is made by an artificial combination of two or more otherwise separated segments of sequence, for example a CMV vector comprising a heterologous antigen. This artificial combination is often accomplished by chemical synthesis or, more commonly, by the artificial manipulation of isolated segments of nucleic acids, e.g., by genetic engineering techniques. A recombinant polypeptide may also refer to a polypeptide that has been made using recombinant nucleic acids, including recombinant nucleic acids transferred to a host organism that is not the natural source of the polypeptide (for example, nucleic acids encoding polypeptides that form a CMV vector comprising a heterologous antigen).

Ortholog: As used herein, the term “ortholog” refers to genes in different species that evolved from a common ancestral gene by speciation. Normally, orthologs retain the same function in the course of evolution. Orthologs of proteins are typically characterized by possession of greater than 75% sequence identity counted over the full-length alignment with the amino acid sequence of specific protein using ALIGN set to default parameters.

Pharmaceutically acceptable carriers: As used herein, a “pharmaceutically acceptable carrier” of use is conventional. Remington's Pharmaceutical Sciences, by E. W. Martin, Mack Publishing Co., Easton, PA, 19th Edition, 1995, describes compositions and formulations suitable for pharmaceutical delivery of the compositions disclosed herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol, or the like as a vehicle. For solid compositions (such as powder, pill, tablet, or capsule forms), conventional non-toxic solid carriers may include, for example, pharmaceutical grades of mannitol, lactose, starch, or magnesium stearate. In addition to biologically neutral carriers, pharmaceutical compositions to be administered may contain minor amounts of non-toxic auxiliary substances, such as wetting or emulsifying agents, preservatives, and pH buffering agents and the like, for example sodium acetate or sorbitan monolaurate.

Polynucleotide: As used herein, the term “polynucleotide” refers to a polymer of ribonucleic acid (RNA) or deoxyribonucleic acid (DNA). A polynucleotide is made up of four bases; adenine, cytosine, guanine, and thymine/uracil (uracil is used in RNA). A coding sequence from a nucleic acid is indicative of the sequence of the protein encoded by the nucleic acid.

Polypeptide: The terms “protein”, “peptide”, “polypeptide”, and “amino acid sequence” are used interchangeably herein to refer to polymers of amino acid residues of any length. The polymer may be linear or branched, it may comprise modified amino acids or amino acid analogs, and it may be interrupted by chemical moieties other than amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling or bioactive component.

Orthologs of proteins are typically characterized by possession of greater than 75% sequence identity counted over the full-length alignment with the amino acid sequence of specific protein using ALIGN set to default parameters. Proteins with even greater similarity to a reference sequence will show increasing percentage identities when assessed by this method, such as at least 80%, at least 85%, at least 90%, at least 92%, at least 95%, or at least 98% sequence identity. In addition, sequence identity can be compared over the full length of particular domains of the disclosed peptides.

Sequence identity/similarity: As used herein, the identity/similarity between two or more nucleic acid sequences, or two or more amino acid sequences, is expressed in terms of the identity or similarity between the sequences. Sequence identity may be measured in terms of percentage identity; the higher the percentage, the more identical the sequences are. Sequence similarity may be measured in terms of percentage identity or similarity (which takes into account conservative amino acid substitutions); the higher the percentage, the more similar the sequences are. Polypeptides or protein domains thereof that have a significant amount of sequence identity and also function the same or similarly to one another (for example, proteins that serve the same functions in different species or mutant forms of a protein that do not change the function of the protein or the magnitude thereof) may be called “homologs.”

Methods of alignment of sequences for comparison are well known in the art. Various programs and alignment algorithms are described in: Smith & Waterman, Adv Appl Math 2, 482 (1981); Needleman & Wunsch, J Mol Biol 48, 443 (1970); Pearson & Lipman, Proc Natl Acad Sci USA 85, 2444 (1988); Higgins & Sharp, Gene 73, 237-244 (1988); Higgins & Sharp, CABIOS 5, 151-153 (1989); Corpet et al, Nuc Acids Res 16, 10881-10890 (1988); Huang et al, Computer App Biosci 8, 155-165 (1992); and Pearson et al, Meth Mol Bio 24, 307-331 (1994). In addition, Altschul et al, J Mol Biol 215, 403-410 (1990), presents a detailed consideration of sequence alignment methods and homology calculations.

The NCBI Basic Local Alignment Search Tool (BLAST) (Altschul et al, (1990) supra) is available from several sources, including the National Center for Biological Information (NCBI, National Library of Medicine, Building 38A, Room 8N805, Bethesda, MD 20894) and on the Internet, for use in connection with the sequence analysis programs blastp, blastn, blastx, tblastn and tblastx. Additional information may be found at the NCBI web site.

BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr database, swissprot database, and patented sequences database. Queries searched with the blastn program are filtered with DUST (Hancock & Armstrong, Comput Appl Biosci 10, 67-70 (1994.) Other programs use SEG. In addition, a manual alignment may be performed. Proteins with even greater similarity will show increasing percentage identities when assessed by this method, such as at least about 75%, 80%, 85%, 90%, 95%, 98%, or 99% sequence identity to a protein.

When aligning short peptides (fewer than around 30 amino acids), the alignment is performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, or 99% sequence identity to a protein. When less than the entire sequence is being compared for sequence identity, homologs will typically possess at least 75% sequence identity over short windows of 10-20 amino acids, and may possess sequence identities of at least 85%, 90%, 95% or 98% depending on their identity to the reference sequence. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence may be made using this degeneracy to produce multiple nucleic acid molecules that all encode substantially the same protein. Such homologous nucleic acid sequences can, for example, possess at least about 50%, 60%, 70%, 80%, 90%, 95%, 98%, or 99% sequence identity to a nucleic acid that encodes a protein.

Subject: As used herein, the term “subject” refers to a living multi-cellular vertebrate organisms, a category that includes both human and non-human mammals.

T cell response: As used herein, the term “T cell response” refers to causing or stimulating a T cell to have a sustained or amplified biological function. For example, induced or enhanced T cell responses include increased production of cytokines by CD8+ T cells, increased proliferation, increased antigen responsiveness, increased persistence, or increased target cell cytotoxicity relative to the response before intervention.

Treatment: As used herein, the term “treatment” refers to an intervention that ameliorates a sign or symptom of a disease or pathological condition. As used herein, the terms “treatment”, “treat” and “treating,” with reference to a disease, pathological condition or symptom, also refers to any observable beneficial effect of the treatment. The beneficial effect may be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease. A prophylactic treatment is a treatment administered to a subject who does not exhibit signs of a disease or exhibits only early signs, for the purpose of decreasing the risk of developing pathology. A therapeutic treatment is a treatment administered to a subject after signs and symptoms of the disease have developed.

Vaccine: An immunogenic composition that can be administered to a mammal, such as a human, to confer immunity, such as active immunity, to a disease or other pathological condition. Vaccines can be used prophylactically or therapeutically. Thus, vaccines can be used to reduce the likelihood of developing a disease (such as a tumor or pathological infection) or to reduce the severity of symptoms of a disease or condition, limit the progression of the disease or condition (such as a tumor or a pathological infection), or limit the recurrence of a disease or condition (such as a tumor). In some aspects, a vaccine is a dissemination-impaired CMV expressing a heterologous antigen, such as a tumor associated antigen derived from a tumor of the lung, prostate, ovary, breast, colon, cervix, liver, kidney, bone, or a melanoma.

Vector: Nucleic acid molecules of particular sequence can be incorporated into a vector that is then introduced into a host cell. A vector may include nucleic acid sequences that permit it to replicate in a host cell, such as an origin of replication. A vector may also include one or more selectable marker genes and other genetic elements known in the art, including promoter elements that direct nucleic acid expression. Vectors can be viral vectors, such as CMV vectors. Viral vectors may be constructed from wild type or attenuated virus, including replication deficient virus, dissemination-impaired virus, and cells containing the viral vectors are used to reconstitute and propagate the vector.

Wild-type: The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene.

### II. Methods for the Treatment and Prevention of a Pathogenic Infection

Disclosed herein are methods for the treatment of a pathogenic infection. In some aspects, the pathogen is an influenza virus. The methods involve administering an effective amount of at least one recombinant CMV vector comprising a) a first nucleic acid sequence encoding an influenza matrix protein, b) a second nucleic acid sequence encoding an influenza nucleoprotein, and a third nucleic acid sequence encoding an influenza polymerase. In some aspects, the influenza polymerase is the PB1 subunit. In some aspects, the first, second, and third nucleic acid sequences are comprised on three different recombinant CMV vectors.

In some aspects, the CMV vector does not express an active UL146 protein or ortholog thereof and does not express an active UL147 protein or ortholog thereof.

In some aspects, the methods also comprise induction of MHC-E restricted T cells. In some aspects, the methods comprise administration the one or more recombinant CMV vectors vector comprising a) a first nucleic acid sequence encoding an influenza matrix protein, b) a second nucleic acid sequence encoding an influenza nucleoprotein, and a third nucleic acid sequence encoding an influenza polymerase.

In some aspects, the recombinant CMV vector does not express an active UL128 protein or ortholog thereof; does not express an active UL 130 protein or ortholog thereof; and does not express an active UL 146 protein or ortholog thereof. In some aspects, the recombinant CMV vector does not express an active UL147 protein or ortholog thereof. In some aspects, the recombinant CMV vector does not express an active UL 18 protein or ortholog thereof. In some aspects, the recombinant CMV vector does not express an active pp71 (UL82) protein or ortholog thereof.

In some aspects, the CMV vector comprises: a) a first nucleic acid sequence encoding an influenza matrix protein; b) a second nucleic acid sequence encoding an influenza nucleoprotein; and c) a third nucleic acid sequence encoding an influenza polymerase; and does not express an active UL 146 protein or ortholog thereof and does not express an active UL147 protein or ortholog thereof.

In some aspects, the CMV vector comprises: a) a first nucleic acid sequence encoding an influenza matrix protein; b) a second nucleic acid sequence encoding an influenza nucleoprotein; and c) a third nucleic acid sequence encoding an influenza polymerase; and does not express an active UL128 protein or ortholog thereof; does not express an active UL 130 protein or ortholog thereof; does not express an active UL146 protein or ortholog thereof; does not express an active UL 147 protein or ortholog thereof; and does not express an active UL 18 protein or ortholog thereof.

In some aspects, the recombinant CMV vector is a human CMV (HCMV) vector, a rhesus macaque CMV (RhCMV) vector, or a cynomolgus macaque CMV (CyCMV) vector. In some aspects, the CMV vector is a human CMV (HCMV) vector. In some aspects, the CMV vector is a rhesus macaque CMV (RhCMV) vector. In some aspects, the CMV vector is a cynomolgus macaque CMV (CyCMV) vector.

In some aspects, the HCMV vector does not express an active UL128 protein or ortholog thereof, does not express an active UL 130 protein or ortholog thereof, does not express an active UL 146 protein or ortholog thereof, does not express an active UL147 protein or ortholog thereof, and does not express an active UL 18 protein or ortholog thereof. In some aspects, the HCMV vector does not express an active UL 128 protein or ortholog thereof, does not express an active UL 130 protein or ortholog thereof, does not express an active UL 146 protein or ortholog thereof, does not express an active UL147 protein or ortholog thereof, does not express an active UL 18 protein or ortholog thereof, and does not express an active pp71 (UL82) protein or ortholog thereof.

In some aspects, the RhCMV vector does not express an active UL128 protein or ortholog thereof, does not express an active UL 130 protein or ortholog thereof, and does not express an active UL 146 protein or ortholog thereof. In some aspects, the RhCMV vector does not express an active UL 128 protein or ortholog thereof, does not express an active UL 130 protein or ortholog thereof, does not express an active UL146 protein or ortholog thereof, and does not express an active pp71 (UL82) protein or ortholog thereof.

In some aspects, the CyCMV vector does not express an active UL 128 protein or ortholog thereof, does not express an active UL130 protein or ortholog thereof, and does not express an active UL 146 protein or ortholog thereof. In some aspects, the CyCMV vector does not express an active UL 128 protein or ortholog thereof, does not express an active UL 130 protein or ortholog thereof, does not express an active UL146 protein or ortholog thereof, and does not express an active pp71 (UL82) protein or ortholog thereof.

The influence matrix protein, influenza nucleoprotein, and influenza polymerase can be from any Influenza A or Influenza B virus subtype. In some aspects, at least one of the influenza matrix protein, influenza nucleoprotein, or influenza polymerase is derived from a H1N1 influenza virus, H1N2 influenza virus, H2N2 influenza virus, H3N2 influenza virus, H5N1 influenza virus, H5N2 influenza virus, H5N9 influenza virus, H7N2 influenza virus, H7N3 influenza virus, H7N7 influenza virus, H7N9 influenza virus, H9N2 influenza virus, H10N7 influenza virus, or H10N3 influenza virus. In some aspects, the influenza matrix protein is derived from a H1N1 influenza virus, H1N2 influenza virus, H2N2 influenza virus, H3N2 influenza virus, H5N1 influenza virus, H5N2 influenza virus, H5N9 influenza virus, H7N2 influenza virus, H7N3 influenza virus, H7N7 influenza virus, H7N9 influenza virus, H9N2 influenza virus, H10N7 influenza virus, or H10N3 influenza virus. In some aspects, the influenza nucleoprotein is derived from a H1N1 influenza virus, H1N2 influenza virus, H2N2 influenza virus, H3N2 influenza virus, H5N1 influenza virus, H5N2 influenza virus, H5N9 influenza virus, H7N2 influenza virus, H7N3 influenza virus, H7N7 influenza virus, H7N9 influenza virus, H9N2 influenza virus, H10N7 influenza virus, or H10N3 influenza virus. In some aspects, the influenza matrix protein is derived from a H1N1 influenza virus, H1N2 influenza virus, H2N2 influenza virus, H3N2 influenza virus, H5N1 influenza virus, H5N2 influenza virus, H5N9 influenza virus, H7N2 influenza virus, H7N3 influenza virus, H7N7 influenza virus, H7N9 influenza virus, H9N2 influenza virus, H10N7 influenza virus, or H10N3 influenza virus. In some aspects, the influenza matrix protein, influenza nucleoprotein, and influenza polymerase are derived from a H1N1 influenza virus, H1N2 influenza virus, H2N2 influenza virus, H3N2 influenza virus, H5N1 influenza virus, H5N2 influenza virus, H5N9 influenza virus, H7N2 influenza virus, H7N3 influenza virus, H7N7 influenza virus, H7N9 influenza virus, H9N2 influenza virus, H10N7 influenza virus, or H10N3 influenza virus. In some aspects the influenza polymerase is a PB1 subunit.

In some aspects, at least one of the influenza matrix protein, influenza nucleoprotein, or influenza polymerase is derived from an H1N1 influenza virus. In some aspects, the influenza matrix protein is derived from a H1N1 influenza virus. In some aspects, the influenza nucleoprotein is derived from a H1N1 influenza virus. In some aspects, the influenza matrix protein is derived from a H1N1 influenza virus. In some aspects, the influenza polymerase, influenza nucleoprotein, and influenza polymerase are derived from a H1N1 influenza virus.

In some aspects, at least one of the influenza matrix protein, influenza nucleoprotein, or influenza polymerase is derived from a 1918 H1N1 influenza virus. In some aspects, the influenza matrix protein is derived from a 1918 H1N1 influenza virus. In some aspects, the influenza nucleoprotein is derived from a 1918 H1N1 influenza virus. In some aspects, the influenza polymerase is derived from a 1918 H1N1 influenza virus. In some aspects, the influenza matrix protein, influenza nucleoprotein, and influenza polymerase are derived from a 1918 H1N1 influenza virus.

In some aspects, the nucleic acid sequence encoding the matrix protein is a wild-type or a codon-optimized nucleic acid sequence. In some aspects, the nucleic acid sequence encoding the matrix protein is a codon-optimized nucleic acid sequence.

In some aspects, the nucleic acid sequence encoding the matrix protein has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the sequence of SEQ ID NOs: 1 or 2. In some aspects, the nucleic acid sequence encoding the matrix protein has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the sequence of SEQ ID NO: 1. In some aspects, the nucleic acid sequence encoding the matrix protein has the sequence of SEQ ID NO: 1.

In some aspects, the matrix protein has an amino acid sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the sequence of SEQ ID NOs: 7 or 8. In some aspects, the matrix protein has an amino acid sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the sequence of SEQ ID NO: 10. In some aspects, matrix protein the amino acid sequence of SEQ ID NO: 10.

In some aspects, the nucleic acid sequence encoding the nucleoprotein is a wild-type or a codon-optimized nucleic acid sequence. In some aspects, the nucleic acid sequence encoding the nucleoprotein has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the sequence of SEQ ID NOs: 3 or 4. In some aspects, the nucleic acid sequence encoding the nucleoprotein has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the sequence of SEQ ID NO: 3. In some aspects, the nucleic acid sequence encoding the nucleoprotein has the sequence of SEQ ID NO: 3.

In some aspects, the nucleoprotein has an amino acid sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the sequence of SEQ ID NOs: 9 or 10. In some aspects, the nucleoprotein has an amino acid sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the sequence of SEQ ID NO: 12. In some aspects, the nucleoprotein has the amino acid sequence of SEQ ID NO: 12.

In some aspects, the nucleic acid sequence encoding the polymerase encodes a polymerase basic 1 (PB1) protein, a polymerase basic 2 (PB2) protein, or a polymerase acidic protein. In some aspects, the nucleic acid sequence encoding the polymerase encodes a polymerase basic 1 (PB1) protein.

In some aspects, the nucleic acid sequence encoding the polymerase is a wild-type or a codon-optimized nucleic acid sequence. In some aspects, the nucleic acid sequence encoding the polymerase is a codon-optimized nucleic acid sequence.

In some aspects, the nucleic acid sequence encoding the polymerase has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the sequence of SEQ ID NOs: 5 or 6. In some aspects, the nucleic acid sequence encoding the polymerase has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the sequence of SEQ ID NO: 5. In some aspects, the nucleic acid sequence encoding the polymerase has the sequence of SEQ ID NO: 5.

In some aspects, the polymerase has an amino acid sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the sequence of SEQ ID NOs: 14 or 15. In some aspects, the polymerase has an amino acid sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the sequence of SEQ ID NO: 14. In some aspects, the polymerase has the amino acid sequence of SEQ ID NO: 14.

TABLE 1
Nucleic Acid Sequences of Vector Inserts 1

| | SEQ | |
|---|---|---|
| | ID | |
| Description | NO: | Sequence |
| Codon- | 1 | atgagcctcctgaccgaggtggagacctatgtgctgtccatcgtg |
| optimized | | cccagcggccctctgaaagccgagatcgcccagagactggaggat |
| Matrix protein | | gtgttcgccggcaagaacaccgacctggaggctctgatggagtgg |
| 1 (M1) | | ctgaagaccaggcctatcctgtcccccctgaccaagggcatcctg |
| | | ggctttgtgttcaccctgaccgtgccctccgaaaggggactgcag |
| | | aggaggaggttcgtgcagaatgccctgaacggcaacggcgaccct |
| | | aacaacatggacagggccgtgaagctgtacaggaagctgaagagg |
| | | gagatcaccttccacggcgccaaggaggtggccctgagctacagc |
| | | gctggagccctggccagctgcatgggcctgatttacaacaggatg |
| | | ggcaccgtgaccaccgaagtggccttcggcctggtgtgcgccacc |
| | | tgtgaacagatcgccgacagccagcataggagccatagacagatg |
| | | gtgaccaccacaaaccccctgatcaggcacgagaacagaatggtg |
| | | ctggcctccaccacagccaaggccatggagcagatggccggctcc |
| | | agcgaacaggctgccgaggccatggaggtggctagccaggccaga |
| | | caaatggtccaagccatgaggaccatcggcacacaccctagcagc |
| | | agcgctggcctgaaggacgatctgatcgagaacctgcaggcctac |
| | | cagaagaggatgggcgtgcagatgcagaggttcaag |
| Codon- | 2 | atgagcctcctgaccgaggtggagacctatgtgctgtccatcgtg |
| optimized | | cccagcggccctctgaaagccgagatcgcccagagactggaggat |
| Matrix protein | | gtgttcgccggcaagaacaccgacctggaggctctgatggagtgg |
| 1 (M1) with | | ctgaagaccaggcctatcctgtcccccctgaccaagggcatcctg |
| V5-tag | | ggctttgtgttcaccctgaccgtgccctccgaaaggggactgcag |
| | | aggaggaggttcgtgcagaatgccctgaacggcaacggcgaccct |
| | | aacaacatggacagggccgtgaagctgtacaggaagctgaagagg |
| | | gagatcaccttccacggcgccaaggaggtggccctgagctacagc |
| | | gctggagccctggccagctgcatgggcctgatttacaacaggatg |
| | | ggcaccgtgaccaccgaagtggccttcggcctggtgtgcgccacc |
| | | tgtgaacagatcgccgacagccagcataggagccatagacagatg |
| | | gtgaccaccacaaaccccctgatcaggcacgagaacagaatggtg |
| | | ctggcctccaccacagccaaggccatggagcagatggccggctcc |
| | | agcgaacaggctgccgaggccatggaggtggctagccaggccaga |
| | | caaatggtccaagccatgaggaccatcggcacacaccctagcagc |
| | | agcgctggcctgaaggacgatctgatcgagaacctgcaggcctac |
| | | cagaagaggatgggcgtgcagatgcagaggttcaagggcaagccc |
| | | attcctaaccctctgctgggcctggactccacatga |
| Codon- | 3 | atggcttcccagggcaccaagaggagctacgagcagatggagacc |
| optimized | | gacggagaaaggcagaacgccaccgaaatcagagccagcgtgggc |
| Nucleoprotein | | aggatgatcggcggcatcggcaggttctacattcagatgtgcacc |
| (NP) | | gagctgaagctgagcgactacgagggcagactgatccagaacagc |
| | | atcaccatcgaaaggatggtcctgtccgctttcgacgagaggagg |
| | | aacaagtacctggaagaacaccccagcgctggcaaggaccccaag |
| | | aagaccggaggccccatctacaggagaatcgacggcaagtggatg |
| | | agagagctcatcctctacgacaaggaggagatcagaaggatctgg |
| | | aggcaggctaataatggcgaagacgccaccgccggactgacccac |
| | | atgatgatttggcacagcaatctgaacgacgctacctaccagagg |
| | | accagggccctcgtgaggacaggcatggaccccagaatgtgctcc |
| | | ctgatgcaaggctccacactgcccaggaggagcggcgccgccggc |
| | | gccgccgtcaaaggcgtgggaaccatggtgatggaactgattagg |
| | | atgattaagaggggaatcaatgacaggaatttctggaggggagag |
| | | aacggcagaaggaccagaatcgcctacgagaggatgtgcaacatc |
| | | ctcaagggcaagttccagaccgccgcccaaagggccatgatggac |
| | | caggtcagggagagcaggaatcctggcaacgccgaaatcgaggac |
| | | ctgattttcctggccaggagcgctctgatcctgaggggctccgtg |
| | | gcccacaagtcctgcctccctgcttgtgtgtacggccctgccgtg |
| | | gctagcggctacgacttcgagagggagggatatagcctggtcgga |
| | | attgaccctttcagactgctgcagaacagccaggtctacagcctg |
| | | atcaggcctaacgagaaccctgcccacaagagccagctggtgtgg |
| | | atggcctgccacagcgccgccttcgaggatctgagggtcagcagc |
| | | ttcatcagaggcacaagagtggtgcctagaggcaagctgtccaca |
| | | aggggcgtgcagatcgccagcaacgagaatatggagaccatggat |
| | | tcctccaccctggaactgaggtccaggtactgggccatcagaacc |
| | | aggtccggcggcaacacaaaccagcagagagccagcgctggccag |
| | | atttccgtgcaacccacattctccgtgcagaggaatctgcccttc |
| | | gagagggccaccatcatggccgctttcaccggaaacaccgaggga |
| | | aggaccagcgatatgaggaccgagatcatcaggatgatggagtcc |
| | | gctaggcccgaggatgtgagcttccagggcaggggagtgttcgaa |
| | | ctgtccgatgagaaagccacaagccctatcgtgccttccttcgac |
| | | atgtccaacgagggctcctacttcttcggcgacaacgccgaggag |
| | | tatgacaacgactataaggaccacgacggcgactacaaggatcac |
| | | gacatc |
| Codon- | 4 | atggcttcccagggcaccaagaggagctacgagcagatggagacc |
| optimized | | gacggagaaaggcagaacgccaccgaaatcagagccagcgtgggc |
| Nucleoprotein | | aggatgatcggcggcatcggcaggttctacattcagatgtgcacc |
| (NP) with | | gagctgaagctgagcgactacgagggcagactgatccagaacagc |
| Flag-tag | | atcaccatcgaaaggatggtcctgtccgctttcgacgagaggagg |
| | | aacaagtacctggaagaacaccccagcgctggcaaggaccccaag |
| | | aagaccggaggccccatctacaggagaatcgacggcaagtggatg |
| | | agagagctcatcctctacgacaaggaggagatcagaaggatctgg |
| | | aggcaggctaataatggcgaagacgccaccgccggactgacccac |
| | | atgatgatttggcacagcaatctgaacgacgctacctaccagagg |
| | | accagggccctcgtgaggacaggcatggaccccagaatgtgctcc |
| | | ctgatgcaaggctccacactgcccaggaggagcggcgccgccggc |
| | | gccgccgtcaaaggcgtgggaaccatggtgatggaactgattagg |
| | | atgattaagaggggaatcaatgacaggaatttctggaggggagag |
| | | aacggcagaaggaccagaatcgcctacgagaggatgtgcaacatc |
| | | ctcaagggcaagttccagaccgccgcccaaagggccatgatggac |
| | | caggtcagggagagcaggaatcctggcaacgccgaaatcgaggac |
| | | ctgattttcctggccaggagcgctctgatcctgaggggctccgtg |
| | | gcccacaagtcctgcctccctgcttgtgtgtacggccctgccgtg |
| | | gctagcggctacgacttcgagagggagggatatagcctggtcgga |
| | | attgaccctttcagactgctgcagaacagccaggtctacagcctg |
| | | atcaggcctaacgagaaccctgcccacaagagccagctggtgtgg |
| | | atggcctgccacagcgccgccttcgaggatctgagggtcagcagc |
| | | ttcatcagaggcacaagagtggtgcctagaggcaagctgtccaca |
| | | aggggcgtgcagatcgccagcaacgagaatatggagaccatggat |
| | | tcctccaccctggaactgaggtccaggtactgggccatcagaacc |
| | | aggtccggcggcaacacaaaccagcagagagccagcgctggccag |
| | | atttccgtgcaacccacattctccgtgcagaggaatctgcccttc |
| | | gagagggccaccatcatggccgctttcaccggaaacaccgaggga |
| | | aggaccagcgatatgaggaccgagatcatcaggatgatggagtcc |
| | | gctaggcccgaggatgtgagcttccagggcaggggagtgttcgaa |
| | | ctgtccgatgagaaagccacaagccctatcgtgccttccttcgac |
| | | atgtccaacgagggctcctacttcttcggcgacaacgccgaggag |
| | | tatgacaacgactataaggaccacgacggcgactacaaggatcac |
| | | gacatcgattataaggacgacgatgataagtga |
| Codon- | 5 | atggacgtcaaccccaccctgctcttcctgaaagtccctgctcag |
| Optimized | | aacgccatctccaccacctttccctatacaggagaccccccctac |
| Polymerase B1 | | agccacggaaccggaaccggatacaccatggacaccgtgaacaga |
| (PB1) | | acccatcagtactccgaaaaaggcaggtggaccaccaacaccgaa |
| | | acaggagccccccagctgaatcctatcgatggccctctccccgag |
| | | gataatgagccctccggctacgcccaaaccgactgtgtgctcgag |
| | | gctatggctttcctggaggagagccaccctggcattttcgagaac |
| | | agctgcctggagaccatggaggtggtgcagcagacaagagtcgat |
| | | aagctcacccaaggaaggcaaacctacgactggaccctcaatagg |
| | | aatcagcctgccgccaccgccctggccaatacaatcgaggtgttc |
| | | aggagcaacggcctcaccgccaatgagtccggcagactgatcgac |
| | | ttcctgaaagacgtgatggagagcatggacaaagaagaaatggag |
| | | atcaccacccattttcagaggaaaagaagggtgagggacaacatg |
| | | acaaagaagatggtgacacagaggaccatcggcaagaaaaagcag |
| | | aggctgaataaaagaagctatctgatcagggctctgaccctcaac |
| | | acaatgaccaaagacgccgaaaggggaaaactcaagaggagggcc |
| | | attgccacccctggaatgcagatcaggggcttcgtctacttcgtc |
| | | gagacactcgccaggtccatttgtgagaaactggagcagagcggc |
| | | ctgcctgtgggggcaacgagaagaaggccaagctggccaacgtgg |
| | | tgaggaaaatgatgacaaatagccaggacacagagctgagcttca |
| | | caatcacaggcgataacaccaagtggaacgagaaccagaacccca |
| | | ggatgttcctcgccatgattacctatattaccaggaaccagcctg |
| | | agtggtttaggaacgtcctgagcatcgctcctatcatgttctcca |
| | | ataagatggccaggctgggaaaaggctatatgttcgagagcaagt |
| | | ccatgaaactgaggacccagatccccgccgagatgctggccagca |
| | | tcgacctgaagtacttcaatgatagcaccaggaagaaaatcgaaa |
| | | agatcagacccctgctcatcgatggcaccgcttccctgtcccccg |
| | | gaatgatgatgggcatgttcaatatgctgagcaccgtgctcggag |
| | | tcagcattctcaacctcggccaaaaaaggtatacaaagacaacct |
| | | actggtgggatggcctgcagagcagcgacgactttgccctgattg |
| | | tgaacgcccccaatcacgagggcatccaggctggcgtcgacagat |
| | | tctacaggacctgcaaactgctgggcattaacatgtccaagaaga |
| | | agagctacatcaacaggacaggcacattcgagttcacctccttct |
| | | tctataggtacggctttgtggccaacttctccatggagctgccct |
| | | ccttcggcgtgtccggcattaatgagagcgccgacatgtccatcg |
| | | gcgtgacagtcatcaagaataacatgatcaataatgacctgggcc |
| | | ctgccacagcccagatggccctgcagctgttcatcaaggattaca |
| | | gatacacatataggtgtcacaggggcgacacccagatccagacca |
| | | gaaggtccttcgagatcaagaagctgtgggagcagacaagaagca |
| | | aggctggactcctggtgagcgacggcggccctaacctgtataaca |
| | | tcagaaatctgcatatccctgaggtgtgtctgaaatgggagctga |
| | | tggacgaggattaccagggcagactctgtaaccccctcaacccct |
| | | ttgtgagccacaaggagatcgaatccgtgaacaacgccgtgatga |
| | | tgcctgcccatggccctgccaagaatatggagtatgatgccgtcg |
| | | ccaccacccacagctggattcccaagaggaataggtccatcctga |
| | | atacaagccagagaggcatcctcgaagatgagcagatgtatcaaa |
| | | aatgctgcaacctgttcgaaaagttcttcccctccagctcctata |
| | | ggaggcctgtgggaatcagcagcatggtggaggctatggtcagca |
| | | gggccaggatcgatgctagaatcgacttcgagagcggcaggatca |
| | | agaaggaagagttcgccgagatcatgaagatttgcagcaccatcg |
| | | aggagctgagaaggcagaag |
| Codon- | 6 | atggacgtcaaccccaccctgctcttcctgaaagtccctgctcag |
| optimized | | aacgccatctccaccacctttccctatacaggagaccccccctac |
| Polymerase B1 | | agccacggaaccggaaccggatacaccatggacaccgtgaacaga |
| (PB1) with | | acccatcagtactccgaaaaaggcaggtggaccaccaacaccgaa |
| HA-tag | | acaggagccccccagctgaatcctatcgatggccctctccccgag |
| | | gataatgagccctccggctacgcccaaaccgactgtgtgctcgag |
| | | gctatggctttcctggaggagagccaccctggcattttcgagaac |
| | | agctgcctggagaccatggaggtggtgcagcagacaagagtcgat |
| | | aagctcacccaaggaaggcaaacctacgactggaccctcaatagg |
| | | aatcagcctgccgccaccgccctggccaatacaatcgaggtgttc |
| | | aggagcaacggcctcaccgccaatgagtccggcagactgatcgac |
| | | ttcctgaaagacgtgatggagagcatggacaaagaagaaatggag |
| | | atcaccacccattttcagaggaaaagaagggtgagggacaacatg |
| | | acaaagaagatggtgacacagaggaccatcggcaagaaaaagcag |
| | | aggctgaataaaagaagctatctgatcagggctctgaccctcaac |
| | | acaatgaccaaagacgccgaaaggggaaaactcaagaggagggcc |
| | | attgccacccctggaatgcagatcaggggcttcgtctacttcgtc |
| | | gagacactcgccaggtccatttgtgagaaactggagcagagcggc |
| | | ctgcctgtgggcggcaacgagaagaaggccaagctggccaacgtg |
| | | gtgaggaaaatgatgacaaatagccaggacacagagctgagcttc |
| | | acaatcacaggcgataacaccaagtggaacgagaaccagaacccc |
| | | aggatgttcctcgccatgattacctatattaccaggaaccagcct |
| | | gagtggtttaggaacgtcctgagcatcgctcctatcatgttctcc |
| | | aataagatggccaggctgggaaaaggctatatgttcgagagcaag |
| | | tccatgaaactgaggacccagatccccgccgagatgctggccagc |
| | | atcgacctgaagtacttcaatgatagcaccaggaagaaaatcgaa |
| | | aagatcagacccctgctcatcgatggcaccgcttccctgtccccc |
| | | ggaatgatgatgggcatgttcaatatgctgagcaccgtgctcgga |
| | | gtcagcattctcaacctcggccaaaaaaggtatacaaagacaacc |
| | | tactggtgggatggcctgcagagcagcgacgactttgccctgatt |
| | | gtgaacgcccccaatcacgagggcatccaggctggcgtcgacaga |
| | | ttctacaggacctgcaaactgctgggcattaacatgtccaagaag |
| | | aagagctacatcaacaggacaggcacattcgagttcacctccttc |
| | | ttctataggtacggctttgtggccaacttctccatggagctgccc |
| | | tccttcggcgtgtccggcattaatgagagcgccgacatgtccatc |
| | | ggcgtgacagtcatcaagaataacatgatcaataatgacctgggc |
| | | cctgccacagcccagatggccctgcagctgttcatcaaggattac |
| | | agatacacatataggtgtcacaggggcgacacccagatccagacc |
| | | agaaggtccttcgagatcaagaagctgtgggagcagacaagaagc |
| | | aaggctggactcctggtgagcgacggcggccctaacctgtataac |
| | | atcagaaatctgcatatccctgaggtgtgtctgaaatgggagctg |
| | | atggacgaggattaccagggcagactctgtaaccccctcaacccc |
| | | tttgtgagccacaaggagatcgaatccgtgaacaacgccgtgatg |
| | | atgcctgcccatggccctgccaagaatatggagtatgatgccgtc |
| | | gccaccacccacagctggattcccaagaggaataggtccatcctg |
| | | aatacaagccagagaggcatcctcgaagatgagcagatgtatcaa |
| | | aaatgctgcaacctgttcgaaaagttcttcccctccagctcctat |
| | | aggaggcctgtgggaatcagcagcatggtggaggctatggtcagc |
| | | agggccaggatcgatgctagaatcgacttcgagagcggcaggatc |
| | | aagaaggaagagttcgccgagatcatgaagatttgcagcaccatc |
| | | gaggagctgagaaggcagaagtatccctacgatgtgcccgactat |
| | | gcctga |
| Influenza A | 7 | Atgagtcttttaaccgaggtcgaaacgtacgttctctctatcgtc |
| virus | | ccgtcaggccccctcaaagccgagatcgcgcagagacttgaagat |
| (A/Brevig_Mis | | gtctttgcagggaagaacaccgatcttgaggctctcatggaatgg |
| sion/1/1918 | | ctaaagacaagaccaatcctgtcacctctgactaaggggatttta |
| (H1N1)) | | ggatttgtgttcacgctcaccgtgcccagtgagcgaggactgcag |
| matrix protein | | cgtagacgctttgtccaaaatgcccttaatgggaacggggatcca |
| 1 (M1) | | aataacatggacagagcagttaaactgtacaggaagcttaagagg |
| | | gagataacattccatggggccaaagaagtagcactcagttattcc |
| | | gctggtgcacttgccagttgtatgggcctcatatacaacaggatg |
| | | gggactgtgaccactgaagtggcatttggcctggtatgcgcaacc |
| | | tgtgaacagattgctgattcccagcatcggtctcacaggcaaatg |
| | | gtgacaacaaccaatccactaatcagacatgagaacagaatggta |
| | | ctggccagcactacggctaaggctatggagcaaatggctggatcg |
| | | agtgagcaagcagcagaggccatggaggttgctagtcaggctagg |
| | | caaatggtgcaggcgatgagaaccattgggactcatcctagctcc |
| | | agtgctggtctgaaagacgatcttattgaaaatttgcaggcctac |
| | | cagaaacgaatgggggtgcagatgcaacgattcaagtgatcctct |
| | | cgttattgccgcaagtatcattgggatcttgcacttgatattgtg |
| | | gattcttgatcgtctttttttcaaatgcatttatcgtcgccttaa |
| | | atacggtttgaaaagagggccttctacggaaggagtgccggagtc |
| | | tatgagggaagaatatcgaaaggaacagcagagtgctgtggatgt |
| | | tgacgatggtcattttgtcaacatagagctggagtaa |
| Influenza A | 8 | atggcgtctcaaggcaccaaacgatcttacgaacagatggagact |
| virus | | gatggggaacgccagaatgccactgaaatcagagcatctgtcgga |
| (A/Brevig | | agaatgattggtggaattggacgattctacatccaaatgtgcacc |
| Mission/1/191 | | gaacttaaactcagtgattatgagggacggctgatccagaacagc |
| 8(H1N1)) | | ataacaatagagagaatggtgctctctgcttttgacgagaggagg |
| nucleoprotein | | aataaatatctggaagaacatcccagcgctgggaaagaccctaag |
| (NP) | | aaaactggaggacccatatacaggagaatagatggaaagtggatg |
| | | agagaactcatcctttatgacaaagaagaaataaggagaatctgg |
| | | cgccaagctaataatggtgaagatgcaacagctggtctgactcac |
| | | atgatgatctggcattccaatttgaatgatgccacttatcagagg |
| | | acaagagctcttgttcgcaccggaatggatcccaggatgtgctct |
| | | ctgatgcaaggttcaactctccctaggaggtctggagccgcaggt |
| | | gctgcagtcaaaggagttggaacaatggtgatggagttgatcagg |
| | | atgatcaaacgtgggatcaatgatcggaacttctggaggggtgag |
| | | aatggacgaaggacaagaattgcctatgaaagaatgtgcaacatt |
| | | ctcaaagggaaatttcaaacagctgcacagagagcaatgatggat |
| | | caagtgagagagagccggaatccaggaaatgctgaaattgaagat |
| | | ctcatctttctggcacggtctgcactcatattgagagggtcagtt |
| | | gctcacaagtcctgcctgcctgcctgtgtgtatggacctgctgta |
| | | gccagtggatacgactttgaaagagagggatactctctggtcgga |
| | | atagaccctttcagactgcttcaaaacagccaagtatacagtcta |
| | | atcagaccaaatgagaatccagcacacaagagtcaactggtatgg |
| | | atggcatgccattctgctgcatttgaagatctgagagtatcaagc |
| | | ttcatcagagggacaagagtggttccaagagggaagctttccact |
| | | agaggggttcagattgcttccaatgaaaacatggagactatggac |
| | | tccagtacccttgaactgagaagcagatactgggccataaggacc |
| | | agaagtggaggaaacactaatcaacagagggcatctgcgggccaa |
| | | atcagcgtgcaacctacattctcagtacagagaaacctccctttt |
| | | gagagagcaaccattatggcagcattcactgggaacacagagggg |
| | | agaacatctgacatgagaaccgaaatcataaggatgatggaaagt |
| | | gcaagaccagaagatgtgtctttccaggggggggagtcttcgagc |
| | | tctcggacgaaaaggcaacgagcccgatcgtgccttcctttgaca |
| | | tgagtaatgaaggatcttatttcttcggagacaatgcagaggagt |
| | | acgacaattaa |
| Influenza A | 9 | Atggatgtcaatccgactttacttttcttgaaagtgccagcgcaa |
| Virus | | aatgctataagcacaacgttcccttatactggagaccctccttac |
| (A/Brevig | | agccatgggacaggaacaggatacaccatggatactgtcaacagg |
| Mission/1/191 | | acacatcagtactcagaaaagggaagatggacaacaaacaccgag |
| 8(H1N1)) | | gacaatgaaccaagtggttatgcccaaacagattgtgtattggaa |
| Polymerase | | actggagcaccacaactcaacccgattgatggaccactgccagaa |
| PB1 (PB1) | | gcaatggctttccttgaggagtcccaccccggtatctttgaaaac |
| | | tcgtgtcttgaaacgatggaggttgttcagcaaacacgagtggac |
| | | aagctgacgcaaggccgacagacctatgactggactctaaatagg |
| | | aaccagcctgctgcaacagcattggccaacacaatagaagtgttc |
| | | agatcaaatggcctcacggccaatgaatctggaaggctcatagac |
| | | ttcctcaaggatgtcatggagtcaatggacaaagaagaaatggaa |
| | | atcacaactcattttcagagaaagagaagagtgagagacaacatg |
| | | actaagaaaatggtgacacagagaacaataggtaaaaagaagcag |
| | | aggttgaacaaaaggagttatctaatcagggcactgaccctgaac |
| | | acaatgaccaaagatgctgagagagggaagctaaaacggagagca |
| | | attgcaaccccagggatgcaaatacgaggatttgtatactttgtt |
| | | gagacactggcaaggagtatatgtgagaaacttgaacaatcagga |
| | | ttgccagttggaggtaatgagaagaaagccaagttggcaaatgtt |
| | | gtaaggaagatgatgaccaattctcaggacactgagctttctttc |
| | | accatcactggagataacaccaaatggaacgaaaatcagaatcct |
| | | cggatgtttttggccatgatcacatacataaccagaaatcagccc |
| | | gaatggttcagaaatgttctaagtattgctccaataatgttctca |
| | | aacaaaatggcgagactggggaaggggtacatgttcgagagcaag |
| | | agtatgaaacttagaactcaaatacctgcagagatgctagcaagc |
| | | atcgatttgaaatatttcaatgattcaacaagaaagaaaattgaa |
| | | aaaatccgaccgctcttaatagatgggactgcatcattgagccct |
| | | ggaatgatgatgggcatgttcaatatgttaagcactgtattaggc |
| | | gtctccatcctgaatcttggacaaaagagatacaccaagactact |
| | | tactggtgggatggtcttcaatcttctgatgattttgctctgatt |
| | | gtgaatgcacccaatcatgagggaattcaagccggagtcgacagg |
| | | ttttatcgaacctgtaagctgcttggaatcaatatgagcaagaaa |
| | | aagtcttacataaacagaacaggcacatttgaattcacaagtttt |
| | | ttctatcgctatgggtttgttgccaatttcagcatggagcttccc |
| | | agctttggggtgtctgggatcaacgagtctgcggacatgagtatt |
| | | ggagttactgtcatcaaaaacaatatgataaacaatgatcttggt |
| | | ccagcaacagctcaaatggcccttcagttgttcatcaaagattac |
| | | aggtacacgtaccggtgccacaggggtgacacccaaatacaaacc |
| | | cgaagatcatttgaaataaagaaattgtgggagcaaacccgttcc |
| | | aaagctggactgctggtctccgacggaggccccaatttgtacaac |
| | | attagaaatctccacattcctgaagtctgcttgaaatgggaattg |
| | | atggatgaggattaccaggggcgtttatgcaacccactgaaccca |
| | | tttgtcagccataaagaaattgaatcagtgaataatgcagtgatg |
| | | atgccagcacatggtccagccaaaaacatggagtatgatgctgtt |
| | | gcaacaacacactcctggattcccaaaagaaatcgatccatcttg |
| | | aacacaagccagagaggaatacttgaagatgaacaaatgtaccaa |
| | | aaatgctgcaatttatttgaaaaattcttccccagcagttcatac |
| | | aggagaccagtcgggatttccagtatggtggaggccatggtttct |
| | | agggcccgaattgatgcacggattgatttcgaatctggaaggata |
| | | aagaaagaggagttcgctgagatcatgaagatctgttccaccatt |
| | | gaagagctcagacggcaaaagtag |

TABLE 2
Amino Acid Sequences of Vector Inserts

| | SEQ ID | |
|---|---|---|
| Description | NO | Sequence |
| Matrix protein | 10 | MSLLTEVETYVLSIVPSGPLKAEIAQRLED |
| | | VFAGKNTDLEALMEWLKTRPILSPLTKGIL |
| | | GFVFTLTVPSERGLQRRRFVQNALNGNGDP |
| | | NNMDRAVKLYRKLKREITFHGAKEVALSYS |
| | | AGALASCMGLIYNRMGTVTTEVAFGLVCAT |
| | | CEQIADSQHRSHRQMVTTTNPLIRHENRMV |
| | | LASTTAKAMEQMAGSSEQAAEAMEVASQAR |
| | | QMVQAMRTIGTHPSSSAGLKDDLIENLQAY |
| | | QKRMGVQMQRFK |
| Matrix protein | 11 | MSLLTEVETYVLSIVPSGPLKAEIAQRLED |
| with V5-tag | | VFAGKNTDLEALMEWLKTRPILSPLTKGIL |
| | | GFVFTLTVPSERGLQRRRFVQNALNGNGDP |
| | | NNMDRAVKLYRKLKREITFHGAKEVALSYS |
| | | AGALASCMGLIYNRMGTVTTEVAFGLVCAT |
| | | CEQIADSQHRSHRQMVTTTNPLIRHENRMV |
| | | LASTTAKAMEQMAGSSEQAAEAMEVASQAR |
| | | QMVQAMRTIGTHPSSSAGLKDDLIENLQAY |
| | | QKRMGVQMQRFKGKPIPNPLLGLDST |
| Nucleoprotein | 12 | MASQGTKRSYEQMETDGERQNATEIRASVG |
| | | RMIGGIGRFYIQMCTELKLSDYEGRLIQNS |
| | | ITIERMVLSAFDERRNKYLEEHPSAGKDPK |
| | | KTGGPIYRRIDGKWMRELILYDKEEIRRIW |
| | | RQANNGEDATAGLTHMMIWHSNLNDATYQR |
| | | TRALVRTGMDPRMCSLMQGSTLPRRSGAAG |
| | | AAVKGVGTMVMELIRMIKRGINDRNFWRGE |
| | | NGRRTRIAYERMCNILKGKFQTAAQRAMMD |
| | | QVRESRNPGNAEIEDLIFLARSALILRGSV |
| | | AHKSCLPACVYGPAVASGYDFEREGYSLVG |
| | | IDPFRLLQNSQVYSLIRPNENPAHKSQLVW |
| | | MACHSAAFEDLRVSSFIRGTRVVPRGKLST |
| | | RGVQIASNENMETMDSSTLELRSRYWAIRT |
| | | RSGGNTNQQRASAGQISVQPTFSVQRNLPF |
| | | ERATIMAAFTGNTEGRTSDMRTEIIRMMES |
| | | ARPEDVSFQGRGVFELSDEKATSPIVPSFD |
| | | MSNEGSYFFGDNAEEYDNDYKDHDGDYKDH |
| | | DI |
| Nucleoprotein | 13 | MASQGTKRSYEQMETDGERQNATEIRASVG |
| with Flag-tag | | RMIGGIGRFYIQMCTELKLSDYEGRLIQNS |
| | | ITIERMVLSAFDERRNKYLEEHPSAGKDPK |
| | | KTGGPIYRRIDGKWMRELILYDKEEIRRIW |
| | | RQANNGEDATAGLTHMMIWHSNLNDATYQR |
| | | TRALVRTGMDPRMCSLMQGSTLPRRSGAAG |
| | | AAVKGVGTMVMELIRMIKRGINDRNFWRGE |
| | | NGRRTRIAYERMCNILKGKFQTAAQRAMMD |
| | | QVRESRNPGNAEIEDLIFLARSALILRGSV |
| | | AHKSCLPACVYGPAVASGYDFEREGYSLVG |
| | | IDPFRLLQNSQVYSLIRPNENPAHKSQLVW |
| | | MACHSAAFEDLRVSSFIRGTRVVPRGKLST |
| | | RGVQIASNENMETMDSSTLELRSRYWAIRT |
| | | RSGGNTNQQRASAGQISVQPTFSVQRNLPF |
| | | ERATIMAAFTGNTEGRTSDMRTEIIRMMES |
| | | ARPEDVSFQGRGVFELSDEKATSPIVPSFD |
| | | MSNEGSYFFGDNAEEYDNDYKDHDGDYKDH |
| | | DIDYKDDDDK |
| Polymerase | 14 | MDVNPTLLFLKVPAQNAISTTFPYTGDPPY |
| B1 | | SHGTGTGYTMDTVNRTHQYSEKGRWTTNTE |
| | | TGAPQLNPIDGPLPEDNEPSGYAQTDCVLE |
| | | AMAFLEESHPGIFENSCLETMEVVQQTRVD |
| | | KLTQGRQTYDWTLNRNQPAATALANTIEVF |
| | | RSNGLTANESGRLIDFLKDVMESMDKEEME |
| | | ITTHFQRKRRVRDNMTKKMVTQRTIGKKKQ |
| | | RLNKRSYLIRALTLNTMTKDAERGKLKRRA |
| | | IATPGMQIRGFVYFVETLARSICEKLEQSG |
| | | LPVGGNEKKAKLANVVRKMMTNSQDTELSF |
| | | TITGDNTKWNENQNPRMFLAMITYITRNQP |
| | | EWFRNVLSIAPIMFSNKMARLGKGYMFESK |
| | | SMKLRTQIPAEMLASIDLKYFNDSTRKKIE |
| | | KIRPLLIDGTASLSPGMMMGMFNMLSTVLG |
| | | VSILNLGQKRYTKTTYWWDGLQSSDDFALI |
| | | VNAPNHEGIQAGVDRFYRTCKLLGINMSKK |
| | | KSYINRTGTFEFTSFFYRYGFVANFSMELP |
| | | SFGVSGINESADMSIGVTVIKNNMINNDLG |
| | | PATAQMALQLFIKDYRYTYRCHRGDTQIQT |
| | | RRSFEIKKLWEQTRSKAGLLVSDGGPNLYN |
| | | IRNLHIPEVCLKWELMDEDYQGRLCNPLNP |
| | | FVSHKEIESVNNAVMMPAHGPAKNMEYDAV |
| | | ATTHSWIPKRNRSILNTSQRGILEDEQMYQ |
| | | KCCNLFEKFFPSSSYRRPVGISSMVEAMVS |
| | | RARIDARIDFESGRIKKEEFAEIMKICSTI |
| | | EELRRQK |
| Polymerase | 15 | MDVNPTLLFLKVPAQNAISTTFPYTGDPPY |
| B1 with HA- | | SHGTGTGYTMDTVNRTHQYSEKGRWTTNTE |
| tag | | TGAPQLNPIDGPLPEDNEPSGYAQTDCVLE |
| | | AMAFLEESHPGIFENSCLETMEVVQQTRVD |
| | | KLTQGRQTYDWTLNRNQPAATALANTIEVF |
| | | RSNGLTANESGRLIDFLKDVMESMDKEEME |
| | | ITTHFQRKRRVRDNMTKKMVTQRTIGKKKQ |
| | | RLNKRSYLIRALTLNTMTKDAERGKLKRRA |
| | | IATPGMQIRGFVYFVETLARSICEKLEQSG |
| | | LPVGGNEKKAKLANVVRKMMTNSQDTELSF |
| | | TITGDNTKWNENQNPRMFLAMITYITRNQP |
| | | EWFRNVLSIAPIMFSNKMARLGKGYMFESK |
| | | SMKLRTQIPAEMLASIDLKYFNDSTRKKIE |
| | | KIRPLLIDGTASLSPGMMMGMFNMLSTVLG |
| | | VSILNLGQKRYTKTTYWWDGLQSSDDFALI |
| | | VNAPNHEGIQAGVDRFYRTCKLLGINMSKK |
| | | KSYINRTGTFEFTSFFYRYGFVANFSMELP |
| | | SFGVSGINESADMSIGVTVIKNNMINNDLG |
| | | PATAQMALQLFIKDYRYTYRCHRGDTQIQT |
| | | RRSFEIKKLWEQTRSKAGLLVSDGGPNLYN |
| | | IRNLHIPEVCLKWELMDEDYQGRLCNPLNP |
| | | FVSHKEIESVNNAVMMPAHGPAKNMEYDAV |
| | | ATTHSWIPKRNRSILNTSQRGILEDEQMYQ |
| | | KCCNLFEKFFPSSSYRRPVGISSMVEAMVS |
| | | RARIDARIDFESGRIKKEEFAEIMKICSTI |
| | | EELRRQKYPYDVPDYA |

In some aspects, the nucleic acid sequence encoding the matrix protein, the nucleic acid sequence encoding the nucleoprotein, and the nucleic acid sequence encoding the polymerase are under the transcriptional control of a UL82 promoter, CMV promoter, a CMV-gH promoter, a HCMV promoter, a RhCMV promoter, a EF1α promoter, a CAG promoter, or a MCMV promoter. In some aspects, the nucleic acid sequence encoding the matrix protein, the nucleic acid sequence encoding the nucleoprotein, and the nucleic acid sequence encoding the polymerase are under the transcriptional control of a UL82 promoter.

In some aspects, the disclosure relates to a pharmaceutical composition comprising the recombinant CMV vector disclosed herein and a pharmaceutically acceptable carrier.

In some aspects, the disclosure relates to an immunogenic composition comprising the recombinant CMV vector disclosed herein and a pharmaceutically acceptable carrier.

Also disclosed herein are methods of generating an immune response in a subject. In some aspects, the methods comprise administering to the subject the CMV vector, pharmaceutical composition, or immunogenic composition disclosed herein in an amount effective to elicit a CD8+ and/or CD4+ T cell response. In some aspects, the CMV vector, pharmaceutical composition, or immunogenic composition disclosed herein are used in generating an immune response in a subject. In some aspects, the CMV vector, pharmaceutical composition, or immunogenic composition disclosed herein are used in the manufacture of a medicament for use in generating an immune response in a subject.

Also disclosed herein are methods of generating an immune response in a subject to a pathogenic infection. In some aspects, the methods comprise administering to the subject the CMV vector, pharmaceutical composition, or immunogenic composition disclosed herein in an amount effective to elicit a CD8+ and/or CD4+ T cell response to a pathogen. In some aspects, the CMV vector, pharmaceutical composition, or immunogenic composition disclosed herein are used in generating an immune response to a pathogen in a subject. In some aspects, the CMV vector, pharmaceutical composition, or immunogenic composition disclosed herein are used in the manufacture of a medicament for use in generating an immune response to a pathogen in a subject.

Also disclosed herein are methods of treating or preventing a pathogenic infection in a subject. In some aspects, the methods comprise administering to the subject the CMV vector, pharmaceutical composition, or immunogenic composition disclosed herein in an amount effective to elicit a CD8+ and/or CD4+ T cell response to a pathogen. In some aspects, the CMV vector, pharmaceutical composition, or immunogenic composition disclosed herein are used for treating or preventing a pathogenic infection in a subject. In some aspects, the CMV vector, pharmaceutical composition, or immunogenic composition disclosed herein are used in the manufacture of a medicament for use in treating or preventing a pathogenic infection in a subject.

In some aspects, the pathogen is an influenza virus. In some aspects, the influenza virus is a H1N1 influenza virus, H1N2 influenza virus, H2N2 influenza virus, H3N2 influenza virus, H5N1 influenza virus, H5N2 influenza virus, H5N9 influenza virus, H7N2 influenza virus, H7N3 influenza virus, H7N7 influenza virus, H7N9 influenza virus, H9N2 influenza virus, H10N7 influenza virus, or H10N3 influenza virus.

In some aspects, the influenza virus is a H1N1 influenza virus. In some aspects, the influenza virus is a H5N1 influenza virus.

In some aspects, the influenza virus is a H1N2 influenza virus. In some aspects, the influenza virus is a H2N2 influenza virus. In some aspects, the influenza virus is a H3N2 influenza virus. In some aspects, the influenza virus is a H5N2 influenza virus. In some aspects, the influenza virus is a H5N9 influenza virus. In some aspects, the influenza virus is a H7N2 influenza virus. In some aspects, the influenza virus is a H7N3 influenza virus. In some aspects, the influenza virus is a H7N7 influenza virus. In some aspects, the influenza virus is a H7N9 influenza virus. In some aspects, the influenza virus is a H9N2 influenza virus. In some aspects, the influenza virus is a H10N7 influenza virus. In some aspects, the influenza virus is a H10N3 influenza virus.

In some aspects, the pathogen is heterologous to the antigens encoded on the CMV vector. In some aspects, the pathogen is an influenza virus. In some aspects, the influenza virus is a H1N2 influenza virus, H2N2 influenza virus, H3N2 influenza virus, H5N1 influenza virus, H5N2 influenza virus, H5N9 influenza virus, H7N2 influenza virus, H7N3 influenza virus, H7N7 influenza virus, H7N9 influenza virus, H9N2 influenza virus, H10N7 influenza virus, or H10N3 influenza virus.

In some aspects, the influenza virus is a H1N1 influenza virus. In some aspects, the influenza virus is a H5N1 influenza virus.

In some aspects, the influenza virus is a H1N2 influenza virus. In some aspects, the influenza virus is a H2N2 influenza virus. In some aspects, the influenza virus is a H3N2 influenza virus. In some aspects, the influenza virus is a H5N2 influenza virus. In some aspects, the influenza virus is a H5N9 influenza virus. In some aspects, the influenza virus is a H7N2 influenza virus. In some aspects, the influenza virus is a H7N3 influenza virus. In some aspects, the influenza virus is a H7N7 influenza virus. In some aspects, the influenza virus is a H7N9 influenza virus. In some aspects, the influenza virus is a H9N2 influenza virus. In some aspects, the influenza virus is a H10N7 influenza virus. In some aspects, the influenza virus is a H10N3 influenza virus.

In some aspects, at least 10% of the CD8+ and/or CD4+ T cells elicited by the recombinant CMV vector are restricted by MHC-I or an ortholog thereof. In some aspects, at least 20%, at least 30%, at least 40%, at least 50%, at least 60%, at least 70%, at least 75%, at least 80%, at least 85%, at least 90%, or at least 95% of the CD8+ and/or CD4+ T cells elicited by the recombinant CMV vector are restricted by MHC-I or an ortholog thereof.

In some aspects, at least 10% of the CD8+ and/or CD4+ T cells elicited by the recombinant CMV vector are restricted by MHC-E or an ortholog thereof. In some aspects, at least 20%, at least 30%, at least 40%, at least 50%, at least 60%, at least 70%, at least 75%, at least 80%, at least 85%, at least 90%, or at least 95% of the CD8+ and/or CD4+ T cells elicited by the recombinant CMV vector are restricted by MHC-E or an ortholog thereof.

In some aspects, at least 10% of the CD8+ and/or CD4+ T cells elicited by the recombinant CMV vector are restricted by MHC-II or an ortholog thereof. In some aspects, at least 20%, at least 30%, at least 40%, at least 50%, at least 60%, at least 70%, at least 75%, at least 80%, at least 85%, at least 90%, or at least 95% of the CD8+ and/or CD4+ T cells elicited by the recombinant CMV vector are restricted by MHC-II or an ortholog thereof.

In some aspects, fewer than 10%, fewer than 20%, fewer than 30%, fewer than 40%, or fewer than 50% of the CD8+ and/or CD4+ T cells elicited by the recombinant CMV vector are restricted by MHC-I or an ortholog thereof. In some aspects, fewer than 10% of the CD8+ and/or CD4+ T cells elicited by the recombinant CMV vector are restricted by MHC-I or an ortholog thereof.

In some aspects, the subject is a human or non-human primate.

In some aspects, administering the CMV vector comprises subcutaneous, intravenous, intramuscular, intraperitoneal, or oral administration of the CMV vector.

In some aspects, the method further comprises identifying a TCR from the CD8+ and/or CD4+ T cells elicited by the CMV vector, wherein the TCR recognizes a MHC-E/influenza antigen-derived peptide complex. In some aspects, the method further comprises identifying a TCR from the CD8+ and/or CD4+ T cells elicited by the CMV vector, wherein the TCR recognizes a MHC-II/influenza antigen-derived peptide complex. In some aspects, the method further comprises identifying a TCR from the CD8+ and/or CD4+ T cells elicited by the CMV vector, wherein the TCR recognizes a MHC-I/influenza antigen-derived peptide complex.

In some aspects, the TCR is identified by DNA or RNA sequencing.

Also disclosed herein are methods of generating CD8+ and/or CD4+ T cells that recognize MHC-E-peptide complexes, the method comprising: (1) administering to a subject the CMV vector as disclosed herein in an amount effective to generate a set of CD8+ and/or CD4+ T cells that recognize MHC-E/peptide complexes; (2) identifying a first TCR from the set of CD8+ and/or CD4+ T cells, wherein the first TCR recognizes a MHC-E/influenza antigen-derived peptide complex; (3) isolating one or more CD8+ and/or CD4+ T cells from the subject; and (4) transfecting the one or more CD8+ and/or CD4+ T cells with an expression vector, wherein the expression vector comprises a nucleic acid sequence encoding a second TCR and a promoter operably linked to the nucleic acid sequence encoding the second TCR, wherein the second TCR comprises CDR3α and CDR3β of the first TCR, thereby generating one or more transfected CD8+ and/or CD4+ T cells that recognize a MHC-E/influenza antigen-derived peptide complex.

In some aspects, the first TCR is identified by DNA or RNA sequencing. In some aspects, the second TCR comprises CDR1α, CDR2α, CDR3α, CDR1β, CDR2β, and CDR3β of the first TCR. In some aspects, the nucleic acid sequence encoding the second TCR is identical to the nucleic acid sequence encoding the first TCR. In some aspects, administering the CMV vector to the subject comprises intravenous, intramuscular, intraperitoneal, or oral administration of the CMV vector to the subject. In some aspects, the subject is a human or nonhuman primate. In some aspects, the method further comprises administering the transfected CD8+ and/or CD4+ T cells to the subject to treat a pathogenic infection. In some aspects, the pathogen is an influenza virus.

In some aspects, the pathogen is an influenza virus. In some aspects, the influenza virus is a H1N1 influenza virus, H1N2 influenza virus, H2N2 influenza virus, H3N2 influenza virus, H5N1 influenza virus, H5N2 influenza virus, H5N9 influenza virus, H7N2 influenza virus, H7N3 influenza virus, H7N7 influenza virus, H7N9 influenza virus, H9N2 influenza virus, H10N7 influenza virus, or H10N3 influenza virus.

In some aspects, the influenza virus is a H1N1 influenza virus. In some aspects, the influenza virus is a H5N1 influenza virus.

In some aspects, the influenza virus is a H1N2 influenza virus. In some aspects, the influenza virus is a H2N2 influenza virus. In some aspects, the influenza virus is a H3N2 influenza virus. In some aspects, the influenza virus is a H5N2 influenza virus. In some aspects, the influenza virus is a H5N9 influenza virus. In some aspects, the influenza virus is a H7N2 influenza virus. In some aspects, the influenza virus is a H7N3 influenza virus. In some aspects, the influenza virus is a H7N7 influenza virus. In some aspects, the influenza virus is a H7N9 influenza virus. In some aspects, the influenza virus is a H9N2 influenza virus. In some aspects, the influenza virus is a H10N7 influenza virus. In some aspects, the influenza virus is a H10N3 influenza virus.

In some aspects, the pathogen is heterologous to the antigens encoded on the CMV vector. In some aspects, the pathogen is an influenza virus. In some aspects, the influenza virus is a H1N2 influenza virus, H2N2 influenza virus, H3N2 influenza virus, virus, H7N3 influenza virus, H7N7 influenza virus, H7N9 influenza virus, H9N2 influenza virus, H10N7 influenza virus, or H10N3 influenza virus.

In some aspects, the influenza virus is a H1N1 influenza virus. In some aspects, the influenza virus is a H5N1 influenza virus.

In some aspects, the influenza virus is a H1N2 influenza virus. In some aspects, the influenza virus is a H2N2 influenza virus. In some aspects, the influenza virus is a H3N2 influenza virus. In some aspects, the influenza virus is a H5N2 influenza virus. In some aspects, the influenza virus is a H5N9 influenza virus. In some aspects, the influenza virus is a H7N2 influenza virus. In some aspects, the influenza virus is a H7N3 influenza virus. In some aspects, the influenza virus is a H7N7 influenza virus. In some aspects, the influenza virus is a H7N9 influenza virus. In some aspects, the influenza virus is a H9N2 influenza virus. In some aspects, the influenza virus is a H10N7 influenza virus. In some aspects, the influenza virus is a H10N3 influenza virus.

Also disclosed herein are methods of generating CD8+ and/or CD4+ T cells that recognize MHC-E-peptide complexes, the method comprising: (1) administering to a first subject the CMV vector as disclosed herein in an amount effective to generate a set of CD8+ and/or CD4+ T cells that recognize MHC-E/peptide complexes; (2) identifying a first TCR from the set of CD8+ and/or CD4+ T cells, wherein the first TCR recognizes a MHC-E/influenza antigen-derived peptide complex; (3) isolating one or more CD8+ and/or CD4+ T cells from a second subject; and (4) transfecting the one or more CD8+ and/or CD4+ T cells with an expression vector, wherein the expression vector comprises a nucleic acid sequence encoding a second TCR and a promoter operably linked to the nucleic acid sequence encoding the second TCR, wherein the second TCR comprises CDR3α and CDR3β of the first TCR, thereby generating one or more transfected CD8+ and/or CD4+ T cells that recognize a MHC-E/influenza antigen-derived peptide complex.

In some aspects, the first TCR is identified by DNA or RNA sequencing. In some aspects, the second TCR comprises CDR1α, CDR2α, CDR3α, CDR1β, CDR2β, and CDR3β of the first TCR. In some aspects, the nucleic acid sequence encoding the second TCR is identical to the nucleic acid sequence encoding the first TCR. In some aspects, the method comprises administering the CMV vector to the subject comprises intravenous, intramuscular, intraperitoneal, or oral administration of the CMV vector to the subject. In some aspects, the subject is a human or nonhuman primate. In some aspects, the method further comprises administering the transfected CD8+ and/or CD4+ T cells to the subject to treat a pathogenic infection.

In some aspects, the pathogen is an influenza virus. In some aspects, the influenza virus is a H1N1 influenza virus, H1N2 influenza virus, H2N2 influenza virus, H3N2 influenza virus, H5N1 influenza virus, H5N2 influenza virus, H5N9 influenza virus, H7N2 influenza virus, H7N3 influenza virus, H7N7 influenza virus, H7N9 influenza virus, H9N2 influenza virus, H10N7 influenza virus, or H10N3 influenza virus.

In some aspects, the influenza virus is a H1N1 influenza virus. In some aspects, the influenza virus is a H5N1 influenza virus.

In some aspects, the influenza virus is a H1N2 influenza virus. In some aspects, the influenza virus is a H2N2 influenza virus. In some aspects, the influenza virus is a H3N2 influenza virus. In some aspects, the influenza virus is a H5N2 influenza virus. In some aspects, the influenza virus is a H5N9 influenza virus. In some aspects, the influenza virus is a H7N2 influenza virus. In some aspects, the influenza virus is a H7N3 influenza virus. In some aspects, the influenza virus is a H7N7 influenza virus. In some aspects, the influenza virus is a H7N9 influenza virus. In some aspects, the influenza virus is a H9N2 influenza virus. In some aspects, the influenza virus is a H10N7 influenza virus. In some aspects, the influenza virus is a H10N3 influenza virus.

In some aspects, the pathogen is heterologous to the antigens encoded on the CMV vector. In some aspects, the pathogen is an influenza virus. In some aspects, the influenza virus is a H1N2 influenza virus, H2N2 influenza virus, H3N2 influenza virus, H5N1 influenza virus, H5N2 influenza virus, H5N9 influenza virus, H7N2 influenza virus, H7N3 influenza virus, H7N7 influenza virus, H7N9 influenza virus, H9N2 influenza virus, H10N7 influenza virus, or H10N3 influenza virus.

In some aspects, the influenza virus is a H1N1 influenza virus. In some aspects, the influenza virus is a H5N1 influenza virus.

In some aspects, the influenza virus is a H1N2 influenza virus. In some aspects, the influenza virus is a H2N2 influenza virus. In some aspects, the influenza virus is a H3N2 influenza virus. In some aspects, the influenza virus is a H5N2 influenza virus. In some aspects, the influenza virus is a H5N9 influenza virus. In some aspects, the influenza virus is a H7N2 influenza virus. In some aspects, the influenza virus is a H7N3 influenza virus. In some aspects, the influenza virus is a H7N7 influenza virus. In some aspects, the influenza virus is a H7N9 influenza virus. In some aspects, the influenza virus is a H9N2 influenza virus. In some aspects, the influenza virus is a H10N7 influenza virus. In some aspects, the influenza virus is a H10N3 influenza virus.

In some aspects, the first TCR is identified by DNA or RNA sequencing. In some aspects, the first subject is a human or nonhuman primate. In some aspects, the second subject is a human or nonhuman primate. In some aspects, the first subject is a non-human primate and the second subject is a human and wherein the second TCR is a chimeric non-human primate-human TCR comprising the non-human primate CDR3α and CDR3β of the first TCR. In some aspects, the second TCR comprises the non-human primate CDR1α, CDR2α, CDR3α, CDR1β, CDR2β, and CDR3β of the first TCR. In some aspects, the nucleic acid sequence encoding the second TCR is identical to the nucleic acid sequence encoding the first TCR. In some aspects, the second TCR is a chimeric TCR. In some aspects, the nucleic acid sequence encoding the second TCR is identical to the nucleic acid sequence encoding the first TCR. In some aspects, the method further comprises administering the CMV vector to the subject comprises intravenous, intramuscular, intraperitoneal, or oral administration of the CMV vector to the subject. In some aspects, the method further comprises administering the transfected CD8+ and/or CD4+ T cells to the subject to treat a pathogenic infection.

In some aspects, the pathogen is an influenza virus. In some aspects, the influenza virus is a H1N1 influenza virus, H1N2 influenza virus, H2N2 influenza virus, H3N2 influenza virus, H5N1 influenza virus, H5N2 influenza virus, H5N9 influenza virus, H7N2 influenza virus, H7N3 influenza virus, H7N7 influenza virus, H7N9 influenza virus, H9N2 influenza virus, H10N7 influenza virus, or H10N3 influenza virus.

In some aspects, the influenza virus is a H1N1 influenza virus. In some aspects, the influenza virus is a H5N1 influenza virus.

In some aspects, the influenza virus is a H1N2 influenza virus. In some aspects, the influenza virus is a H2N2 influenza virus. In some aspects, the influenza virus is a H3N2 influenza virus. In some aspects, the influenza virus is a H5N2 influenza virus. In some aspects, the influenza virus is a H5N9 influenza virus. In some aspects, the influenza virus is a H7N2 influenza virus. In some aspects, the influenza virus is a H7N3 influenza virus. In some aspects, the influenza virus is a H7N7 influenza virus. In some aspects, the influenza virus is a H7N9 influenza virus. In some aspects, the influenza virus is a H9N2 influenza virus. In some aspects, the influenza virus is a H10N7 influenza virus. In some aspects, the influenza virus is a H10N3 influenza virus.

In some aspects, the pathogen is heterologous to the antigens encoded on the CMV vector. In some aspects, the pathogen is an influenza virus. In some aspects, the influenza virus is a H1N2 influenza virus, H2N2 influenza virus, H3N2 influenza virus, H5N1 influenza virus, H5N2 influenza virus, H5N9 influenza virus, H7N2 influenza virus, H7N3 influenza virus, H7N7 influenza virus, H7N9 influenza virus, H9N2 influenza virus, H10N7 influenza virus, or H10N3 influenza virus.

In some aspects, the influenza virus is a H1N1 influenza virus. In some aspects, the influenza virus is a H5N1 influenza virus.

In some aspects, the influenza virus is a H1N2 influenza virus. In some aspects, the influenza virus is a H2N2 influenza virus. In some aspects, the influenza virus is a H3N2 influenza virus. In some aspects, the influenza virus is a H5N2 influenza virus. In some aspects, the influenza virus is a H5N9 influenza virus. In some aspects, the influenza virus is a H7N2 influenza virus. In some aspects, the influenza virus is a H7N3 influenza virus. In some aspects, the influenza virus is a H7N7 influenza virus. In some aspects, the influenza virus is a H7N9 influenza virus. In some aspects, the influenza virus is a H9N2 influenza virus. In some aspects, the influenza virus is a H10N7 influenza virus. In some aspects, the influenza virus is a H10N3 influenza virus.

Also disclosed herein are methods of generating CD8+ and/or CD4+ T cells that recognize MHC-II-peptide complexes, the method comprising: (1) administering to a subject the CMV vector as disclosed herein in an amount effective to generate a set of CD8+ and/or CD4+ T cells that recognize MHC-II/peptide complexes; (2) identifying a first TCR from the set of CD8+ and/or CD4+ T cells, wherein the first TCR recognizes a MHC-II/influenza antigen-derived peptide complex; (3) isolating one or more CD8+ and/or CD4+ T cells from the subject; and (4) transfecting the one or more CD8+ and/or CD4+ T cells with an expression vector, wherein the expression vector comprises a nucleic acid sequence encoding a second TCR and a promoter operably linked to the nucleic acid sequence encoding the second TCR, wherein the second TCR comprises CDR3α and CDR3β of the first TCR, thereby generating one or more transfected CD8+ and/or CD4+ T cells that recognize a MHC-II/influenza antigen-derived peptide complex.

In some aspects, the first TCR is identified by DNA or RNA sequencing. In some aspects, the second TCR comprises CDR1α, CDR2α, CDR3α, CDR1β, CDR2β, and CDR3β of the first TCR. In some aspects, the nucleic acid sequence encoding the second TCR is identical to the nucleic acid sequence encoding the first TCR. In some aspects, the method further comprises administering the CMV vector to the subject comprises intravenous, intramuscular, intraperitoneal, or oral administration of the CMV vector to the subject. In some aspects, the subject is a human or nonhuman primate. In some aspects, the method further comprises administering the transfected CD8+ and/or CD4+ T cells to the subject to treat a pathogenic infection.

In some aspects, the pathogen is an influenza virus. In some aspects, the influenza virus is a H1N1 influenza virus, H1N2 influenza virus, H2N2 influenza virus, H3N2 influenza virus, H5N1 influenza virus, H5N2 influenza virus, H5N9 influenza virus, H7N2 influenza virus, H7N3 influenza virus, H7N7 influenza virus, H7N9 influenza virus, H9N2 influenza virus, H10N7 influenza virus, or H10N3 influenza virus.

In some aspects, the influenza virus is a H1N1 influenza virus. In some aspects, the influenza virus is a H5N1 influenza virus.

In some aspects, the influenza virus is a H1N2 influenza virus. In some aspects, the influenza virus is a H2N2 influenza virus. In some aspects, the influenza virus is a H3N2 influenza virus. In some aspects, the influenza virus is a H5N2 influenza virus. In some aspects, the influenza virus is a H5N9 influenza virus. In some aspects, the influenza virus is a H7N2 influenza virus. In some aspects, the influenza virus is a H7N3 influenza virus. In some aspects, the influenza virus is a H7N7 influenza virus. In some aspects, the influenza virus is a H7N9 influenza virus. In some aspects, the influenza virus is a H9N2 influenza virus. In some aspects, the influenza virus is a H10N7 influenza virus. In some aspects, the influenza virus is a H10N3 influenza virus.

In some aspects, the pathogen is heterologous to the antigens encoded on the CMV vector. In some aspects, the pathogen is an influenza virus. In some aspects, the influenza virus is a H1N2 influenza virus, H2N2 influenza virus, H3N2 influenza virus, H5N1 influenza virus, H5N2 influenza virus, H5N9 influenza virus, H7N2 influenza virus, H7N3 influenza virus, H7N7 influenza virus, H7N9 influenza virus, H9N2 influenza virus, H10N7 influenza virus, or H10N3 influenza virus.

In some aspects, the influenza virus is a H1N1 influenza virus. In some aspects, the influenza virus is a H5N1 influenza virus.

In some aspects, the influenza virus is a H1N2 influenza virus. In some aspects, the influenza virus is a H2N2 influenza virus. In some aspects, the influenza virus is a H3N2 influenza virus. In some aspects, the influenza virus is a H5N2 influenza virus. In some aspects, the influenza virus is a H5N9 influenza virus. In some aspects, the influenza virus is a H7N2 influenza virus. In some aspects, the influenza virus is a H7N3 influenza virus. In some aspects, the influenza virus is a H7N7 influenza virus. In some aspects, the influenza virus is a H7N9 influenza virus. In some aspects, the influenza virus is a H9N2 influenza virus. In some aspects, the influenza virus is a H10N7 influenza virus. In some aspects, the influenza virus is a H10N3 influenza virus.

Also disclosed herein are methods of generating CD8+ and/or CD4+ T cells that recognize MHC-II-peptide complexes, the method comprising: (1) administering to a first subject the CMV vector as disclosed herein in an amount effective to generate a set of CD8+ and/or CD4+ T cells that recognize MHC-II/peptide complexes; (2) identifying a first TCR from the set of CD8+ and/or CD4+ T cells, wherein the first TCR recognizes a MHC-II/influenza antigen-derived peptide complex; (3) isolating one or more CD8+ and/or CD4+ T cells from a second subject; and (4) transfecting the one or more CD8+ and/or CD4+ cells with an expression vector, wherein the expression vector comprises a nucleic acid sequence encoding a second TCR and a promoter operably linked to the nucleic acid sequence encoding the second TCR, wherein the second TCR comprises CDR3α and CDR3β of the first TCR, thereby generating one or more transfected CD8+ and/or CD4+ T cells that recognize a MHC-II/influenza antigen-derived peptide complex.

In some aspects, the first TCR is identified by DNA or RNA sequencing. In some aspects, the first subject is a non-human primate and the second subject is a human and wherein the second TCR is a chimeric non-human primate-human TCR comprising the non-human primate CDR3α and CDR3β of the first TCR. In some aspects, the second TCR comprises the non-human primate CDR1α, CDR2α, CDR3α, CDR1B, CDR2β, and CDR3β of the first TCR. In some aspects, the nucleic acid sequence encoding the second TCR is identical to the nucleic acid sequence encoding the first TCR. In some aspects, the second TCR is a chimeric TCR. In some aspects, the nucleic acid sequence encoding the second TCR is identical to the nucleic acid sequence encoding the first TCR. In some aspects, the method comprises administering the CMV vector to the subject comprises intravenous, intramuscular, intraperitoneal, or oral administration of the CMV vector to the subject. In some aspects, the method comprises administering the transfected CD8+ and/or CD4+ T cells to the subject to treat a pathogenic infection.

In some aspects, the pathogen is an influenza virus. In some aspects, the influenza virus is a H1N1 influenza virus, H1N2 influenza virus, H2N2 influenza virus, H3N2 influenza virus, H5N1 influenza virus, H5N2 influenza virus, H5N9 influenza virus, H7N2 influenza virus, H7N3 influenza virus, H7N7 influenza virus, H7N9 influenza virus, H9N2 influenza virus, H10N7 influenza virus, or H10N3 influenza virus.

In some aspects, the influenza virus is a H1N1 influenza virus. In some aspects, the influenza virus is a H5N1 influenza virus.

In some aspects, the influenza virus is a H1N2 influenza virus. In some aspects, the influenza virus is a H2N2 influenza virus. In some aspects, the influenza virus is a H3N2 influenza virus. In some aspects, the influenza virus is a H5N2 influenza virus. In some aspects, the influenza virus is a H5N9 influenza virus. In some aspects, the influenza virus is a H7N2 influenza virus. In some aspects, the influenza virus is a H7N3 influenza virus. In some aspects, the influenza virus is a H7N7 influenza virus. In some aspects, the influenza virus is a H7N9 influenza virus. In some aspects, the influenza virus is a H9N2 influenza virus. In some aspects, the influenza virus is a H10N7 influenza virus. In some aspects, the influenza virus is a H10N3 influenza virus.

In some aspects, the pathogen is heterologous to the antigens encoded on the CMV vector. In some aspects, the pathogen is an influenza virus. In some aspects, the influenza virus is a H1N2 influenza virus, H2N2 influenza virus, H3N2 influenza virus, H5N1 influenza virus, H5N2 influenza virus, H5N9 influenza virus, H7N2 influenza virus, H7N3 influenza virus, H7N7 influenza virus, H7N9 influenza virus, H9N2 influenza virus, H10N7 influenza virus, or H10N3 influenza virus.

In some aspects, the influenza virus is a H1N1 influenza virus. In some aspects, the influenza virus is a H5N1 influenza virus.

In some aspects, the influenza virus is a H1N2 influenza virus. In some aspects, the influenza virus is a H2N2 influenza virus. In some aspects, the influenza virus is a H3N2 influenza virus. In some aspects, the influenza virus is a H5N2 influenza virus. In some aspects, the influenza virus is a H5N9 influenza virus. In some aspects, the influenza virus is a H7N2 influenza virus. In some aspects, the influenza virus is a H7N3 influenza virus. In some aspects, the influenza virus is a H7N7 influenza virus. In some aspects, the influenza virus is a H7N9 influenza virus. In some aspects, the influenza virus is a H9N2 influenza virus. In some aspects, the influenza virus is a H10N7 influenza virus. In some aspects, the influenza virus is a H10N3 influenza virus.

Also disclosed herein are methods of generating CD8+ and/or CD4+ T cells that recognize MHC-I-peptide complexes, the method comprising: (1) administering to a subject the CMV vector as disclosed herein in an amount effective to generate a set of CD8+ and/or CD4+ T cells that recognize MHC-I/peptide complexes; (2) identifying a first TCR from the set of CD8+ and/or CD4+ T cells, wherein the first TCR recognizes a MHC-I/influenza antigen-derived peptide complex; (3) isolating one or more CD8+ and/or CD4+ T cells from the subject; and (4) transfecting the one or more CD8+ and/or CD4+ T cells with an expression vector, wherein the expression vector comprises a nucleic acid sequence encoding a second TCR and a promoter operably linked to the nucleic acid sequence encoding the second TCR, wherein the second TCR comprises CDR3α and CDR3β of the first TCR, thereby generating one or more transfected CD8+ and/or CD4+ T cells that recognize a MHC-I/influenza antigen-derived peptide complex.

In some aspects, the first TCR is identified by DNA or RNA sequencing. In some aspects, the second TCR comprises CDR1α, CDR2α, CDR3α, CDR1β, CDR2β, and CDR3β of the first TCR. In some aspects, the nucleic acid sequence encoding the second TCR is identical to the nucleic acid sequence encoding the first TCR. In some aspects, the method further comprises administering the CMV vector to the subject comprises intravenous, intramuscular, intraperitoneal, or oral administration of the CMV vector to the subject. In some aspects, the subject is a human or nonhuman primate. In some aspects, the method further comprises administering the transfected CD8+ and/or CD4+ T cells to the subject to treat a pathogenic infection.

In some aspects, the pathogen is an influenza virus. In some aspects, the influenza virus is a H1N1 influenza virus, H1N2 influenza virus, H2N2 influenza virus, H3N2 influenza virus, H5N1 influenza virus, H5N2 influenza virus, H5N9 influenza virus, H7N2 influenza virus, H7N3 influenza virus, H7N7 influenza virus, H7N9 influenza virus, H9N2 influenza virus, H10N7 influenza virus, or H10N3 influenza virus.

In some aspects, the influenza virus is a H1N1 influenza virus. In some aspects, the influenza virus is a H5N1 influenza virus.

In some aspects, the influenza virus is a H1N2 influenza virus. In some aspects, the influenza virus is a H2N2 influenza virus. In some aspects, the influenza virus is a H3N2 influenza virus. In some aspects, the influenza virus is a H5N2 influenza virus. In some aspects, the influenza virus is a H5N9 influenza virus. In some aspects, the influenza virus is a H7N2 influenza virus. In some aspects, the influenza virus is a H7N3 influenza virus. In some aspects, the influenza virus is a H7N7 influenza virus. In some aspects, the influenza virus is a H7N9 influenza virus. In some aspects, the influenza virus is a H9N2 influenza virus. In some aspects, the influenza virus is a H10N7 influenza virus. In some aspects, the influenza virus is a H10N3 influenza virus.

In some aspects, the pathogen is heterologous to the antigens encoded on the CMV vector. In some aspects, the pathogen is an influenza virus. In some aspects, the influenza virus is a H1N2 influenza virus, H2N2 influenza virus, H3N2 influenza virus, H5N1 influenza virus, H5N2 influenza virus, H5N9 influenza virus, H7N2 influenza virus, H7N3 influenza virus, H7N7 influenza virus, H7N9 influenza virus, H9N2 influenza virus, H10N7 influenza virus, or H10N3 influenza virus.

In some aspects, the influenza virus is a H1N1 influenza virus. In some aspects, the influenza virus is a H5N1 influenza virus.

In some aspects, the influenza virus is a H1N2 influenza virus. In some aspects, the influenza virus is a H2N2 influenza virus. In some aspects, the influenza virus is a H3N2 influenza virus. In some aspects, the influenza virus is a H5N2 influenza virus. In some aspects, the influenza virus is a H5N9 influenza virus. In some aspects, the influenza virus is a H7N2 influenza virus. In some aspects, the influenza virus is a H7N3 influenza virus. In some aspects, the influenza virus is a H7N7 influenza virus. In some aspects, the influenza virus is a H7N9 influenza virus. In some aspects, the influenza virus is a H9N2 influenza virus. In some aspects, the influenza virus is a H10N7 influenza virus. In some aspects, the influenza virus is a H10N3 influenza virus.

Also disclosed herein are methods of generating CD8+ and/or CD4+ T cells that recognize MHC-I-peptide complexes, the method comprising: (1) administering to a first subject the CMV vector as disclosed herein in an amount effective to generate a set of CD8+ and/or CD4+ T cells that recognize MHC-I/peptide complexes; (2) identifying a first TCR from the set of CD8+ and/or CD4+ T cells, wherein the first TCR recognizes a MHC-I/influenza antigen-derived peptide complex; (3) isolating one or more CD8+ and/or CD4+ T cells from a second subject; and (4) transfecting the one or more CD8+ and/or CD4+ T cells with an expression vector, wherein the expression vector comprises a nucleic acid sequence encoding a second TCR and a promoter operably linked to the nucleic acid sequence encoding the second TCR, wherein the second TCR comprises CDR3α and CDR3β of the first TCR, thereby generating one or more transfected CD8+ and/or CD4+ T cells that recognize a MHC-I/influenza antigen-derived peptide complex.

In some aspects, the first TCR is identified by DNA or RNA sequencing. In some aspects, the first subject is a human or nonhuman primate. In some aspects, the second subject is a human or nonhuman primate. In some aspects, the first subject is a non-human primate and the second subject is a human and wherein the second TCR is a chimeric non-human primate-human TCR comprising the non-human primate CDR3α and CDR3β of the first TCR. In some aspects, the second TCR comprises the non-human primate CDR1α, CDR2α, CDR3α, CDR1β, CDR2β, and CDR3β of the first TCR. In some aspects, the nucleic acid sequence encoding the second TCR is identical to the nucleic acid sequence encoding the first TCR. In some aspects, the second TCR is a chimeric TCR. In some aspects, the nucleic acid sequence encoding the second TCR is identical to the nucleic acid sequence encoding the first TCR. In some aspects, the method further comprises administering the CMV vector to the subject comprises intravenous, intramuscular, intraperitoneal, or oral administration of the CMV vector to the subject. In some aspects, the method further comprises administering the transfected CD8+ and/or CD4+ T cells to the subject to treat a pathogenic infection.

In some aspects, the pathogen is an influenza virus. In some aspects, the influenza virus is a H1N1 influenza virus, H1N2 influenza virus, H2N2 influenza virus, H3N2 influenza virus, H5N1 influenza virus, H5N2 influenza virus, H5N9 influenza virus, H7N2 influenza virus, H7N3 influenza virus, H7N7 influenza virus, H7N9 influenza virus, H9N2 influenza virus, H10N7 influenza virus, or H10N3 influenza virus.

In some aspects, the influenza virus is a H1N1 influenza virus. In some aspects, the influenza virus is a H5N1 influenza virus.

In some aspects, the influenza virus is a H1N2 influenza virus. In some aspects, the influenza virus is a H2N2 influenza virus. In some aspects, the influenza virus is a H3N2 influenza virus. In some aspects, the influenza virus is a H5N2 influenza virus. In some aspects, the influenza virus is a H5N9 influenza virus. In some aspects, the influenza virus is a H7N2 influenza virus. In some aspects, the influenza virus is a H7N3 influenza virus. In some aspects, the influenza virus is a H7N7 influenza virus. In some aspects, the influenza virus is a H7N9 influenza virus. In some aspects, the influenza virus is a H9N2 influenza virus. In some aspects, the influenza virus is a H10N7 influenza virus. In some aspects, the influenza virus is a H10N3 influenza virus.

In some aspects, the pathogen is heterologous to the antigens encoded on the CMV vector. In some aspects, the pathogen is an influenza virus. In some aspects, the influenza virus is a H1N2 influenza virus, H2N2 influenza virus, H3N2 influenza virus, virus, H7N3 influenza virus, H7N7 influenza virus, H7N9 influenza virus, H9N2 influenza virus, H10N7 influenza virus, or H10N3 influenza virus.

In some aspects, the influenza virus is a H1N1 influenza virus. In some aspects, the influenza virus is a H5N1 influenza virus.

In some aspects, the influenza virus is a H1N2 influenza virus. In some aspects, the influenza virus is a H2N2 influenza virus. In some aspects, the influenza virus is a H3N2 influenza virus. In some aspects, the influenza virus is a H5N2 influenza virus. In some aspects, the influenza virus is a H5N9 influenza virus. In some aspects, the influenza virus is a H7N2 influenza virus. In some aspects, the influenza virus is a H7N3 influenza virus. In some aspects, the influenza virus is a H7N7 influenza virus. In some aspects, the influenza virus is a H7N9 influenza virus. In some aspects, the influenza virus is a H9N2 influenza virus. In some aspects, the influenza virus is a H10N7 influenza virus. In some aspects, the influenza virus is a H10N3 influenza virus.

Also disclosed herein is a CD8+ and/or CD4+ T cell generated by the methods disclosed herein. Also disclosed herein are methods of treating or preventing a pathogenic infection in a subject, the method comprising administering a CD8+ and/or CD4+ T cell as disclosed herein. In some aspects, the CD8+ and/or CD4+ T cell is used in the manufacture of a medicament for use in treating or preventing a pathogenic infection in a subject. In some aspects, the CD8+ and/or CD4+ T cell is used in treating or preventing a pathogenic infection in a subject.

In some aspects, the pathogen is an influenza virus. In some aspects, the influenza virus is a H1N1 influenza virus, H1N2 influenza virus, H2N2 influenza virus, H3N2 influenza virus, H5N1 influenza virus, H5N2 influenza virus, H5N9 influenza virus, H7N2 influenza virus, H7N3 influenza virus, H7N7 influenza virus, H7N9 influenza virus, H9N2 influenza virus, H10N7 influenza virus, or H10N3 influenza virus.

In some aspects, the influenza virus is a H1N1 influenza virus. In some aspects, the influenza virus is a H5N1 influenza virus.

In some aspects, the influenza virus is a H1N2 influenza virus. In some aspects, the influenza virus is a H2N2 influenza virus. In some aspects, the influenza virus is a H3N2 influenza virus. In some aspects, the influenza virus is a H5N2 influenza virus. In some aspects, the influenza virus is a H5N9 influenza virus. In some aspects, the influenza virus is a H7N2 influenza virus. In some aspects, the influenza virus is a H7N3 influenza virus. In some aspects, the influenza virus is a H7N7 influenza virus. In some aspects, the influenza virus is a H7N9 influenza virus. In some aspects, the influenza virus is a H9N2 influenza virus. In some aspects, the influenza virus is a H10N7 influenza virus. In some aspects, the influenza virus is a H10N3 influenza virus.

In some aspects, the pathogen is heterologous to the antigens encoded on the CMV vector. In some aspects, the pathogen is an influenza virus. In some aspects, the influenza virus is a H1N2 influenza virus, H2N2 influenza virus, H3N2 influenza virus, H5N1 influenza virus, H5N2 influenza virus, H5N9 influenza virus, H7N2 influenza virus, H7N3 influenza virus, H7N7 influenza virus, H7N9 influenza virus, H9N2 influenza virus, H10N7 influenza virus, or H10N3 influenza virus.

In some aspects, the influenza virus is a H1N1 influenza virus. In some aspects, the influenza virus is a H5N1 influenza virus.

In some aspects, the influenza virus is a H1N2 influenza virus. In some aspects, the influenza virus is a H2N2 influenza virus. In some aspects, the influenza virus is a H3N2 influenza virus. In some aspects, the influenza virus is a H5N2 influenza virus. In some aspects, the influenza virus is a H5N9 influenza virus. In some aspects, the influenza virus is a H7N2 influenza virus. In some aspects, the influenza virus is a H7N3 influenza virus. In some aspects, the influenza virus is a H7N7 influenza virus. In some aspects, the influenza virus is a H7N9 influenza virus. In some aspects, the influenza virus is a H9N2 influenza virus. In some aspects, the influenza virus is a H10N7 influenza virus. In some aspects, the influenza virus is a H10N3 influenza virus.

### III. Methods for the Modulation of T Cell Responses by CyCMV Vectors

Also disclosed herein is a recombinant cynomolgus cytomegalovirus (CyCMV) vector comprising at least one heterologous antigen, wherein the vector does not express an active UL128 protein or ortholog thereof; does not express an active UL130 protein or ortholog thereof; and does not express an active UL 146 protein or ortholog thereof. In some aspects, the CyCMV vector does not express an active pp71 protein or ortholog thereof.

In some aspects, the at least one heterologous antigen comprises a pathogen-specific antigen, a tumor antigen, a tissue-specific antigen, or a host self-antigen.

The pathogen may be a bacterial pathogen and the antigen may be a protein derived from the bacterial pathogen. The pathogenic bacteria include, but are not limited to, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsii, Salmonella typhi, Sahnonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Vibrio cholera and Yersinia pestis.

The pathogen may be a parasite and the antigen may be a protein derived from the parasite pathogen. The parasite may be a protozoan organism or a protozoan organism causing a disease such as, but not limited to, Acanthamoeha, Bahesiosis, Balantidiasis, Blastocystosis, Coccidia, Dientamoebiasis, Amoebiasis, Giardia, Isosporiasis, Leishmaniasis, Primary amoebic meningoencephalitis (PAM), Malaria, Rhinosporidiosis, Toxoplasmosis—Parasitic pneumonia, Trichomoniasis, Sleeping sickness and Chagas disease. The parasite may be a helminth organism or worm or a disease caused by a helminth organism such as, but not limited to, Ancylostomiasis/Hoolnvorm, Anisakiasis, Roundworm—Parasitic pneumonia, Roundworm-Baylisascariasis, Tapeworm—Tapeworm infection, Clonorchiasis, Dioctophyme renalis infection, Diphyllobothriasis—tapeworm, Guinea worm—Dracunculiasis, Echinococcosis—tapeworm, Pinworm Enterobiasis, Liver fluke Fasciolosis, Fasciolopsiasis intestinal fluke, Gnathostomiasis, Hymenolepiasis, Loa loa filariasis, Calabar swellings, Mansonelliasis, Filariasis, Metagonimiasis intestinal fluke, River blindness, Chinese Liver Fluke, Paragonimiasis, Lung Fluke, Schistosomiasis bilharzia, bilharziosis or snail fever (all types), intestinal schistosomiasis, urinary schistosomiasis, Schistosomiasis by Schistosoma japonicum, Asian intestinal schistosomiasis, Sparganosis, Strongyloidiasis—Parasitic pneumonia, Beef tapevvorm, Pork tapeworm, Toxocariasis, Trichinosis, Swimmer's itch, whipworm and Elephantiasis Lymphatic filariasis.

The parasite may be an organism or disease caused by an organism such as, but not limited to, parasitic worm, Halzom1 Syndrome, Myiasis, Chigoe flea, Human Botfly and Candiru. The parasite may be an ectoparasite or disease caused by an ectoparasite such as, but not limited to, Bedbug, Head louse Pediculosis, Body louse-Pediculosis, Crab louse—Pediculosis, Demodex—Demodicosis, Scabies, Screwworm and Cochlimnyia.

In some aspects, the pathogen specific antigen is derived from a pathogen selected from the group consisting of human immunodeficiency virus, simian immunodeficiency virus, herpes simplex virus type 1, herpes simplex virus type 2, hepatitis B virus, hepatitis C virus, papillomavirus, Plasmodium parasites, and Mycobacterium tuberculosis.

The antigen may be a protein derived from a tumor. As described herein, cancers or tumors include, but are not limited to, Acute lymphoblastic leukemia; Acute myeloid leukemia; Adrenocortical carcinoma; AIDS-related cancers; AIDS-related lymphoma: Anal cancer; Appendix cancer; Astrocytoma, childhood cerebellar or cerebral; Basal cell carcinoma; Bile duct cancer, extrahepatic: Bladder cancer; Bone cancer, Osteosarcoma/Malignant fibrous histiocytoma; Brainstem glioma; Brain tumor; Brain tumor, cerebellar astrocytoma; Brain tumor, cerebral astrocytoma/malignant glioma; Brain tumor, ependymoma; Brain tumor, medulloblastoma: Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood: Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer: Childhood cancers; Chronic lymphocytic leukemia: Chronic rnyelogenous leukemia: Chronic myeloproliferative disorders; Colon Cancer; Cutaneous T-cell lymphoma: Desmoplastic small round cell tumor; Endometrial cancer; Ependymoma; Esophageal cancer; Ewing's sarcoma in the Ewing family of tumors; Extracranial germ cell tumor, Childhood: Extragonadal Germ cell tumor: Extrahepatic bile duct cancer; Eye Cancer, Intraocular melanoma; Eye Cancer, Retinoblastoma; Gallbladder cancer; Gastric (Stomach) cancer; Gastrointestinal Carcinoid Tumor; Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid: Hairy cell leukemia; Head and neck cancer; Hemi cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma: Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia): Leukemia, chronic lymphocytic (also called chronic lymphocytic leukemia); Leukemia, chronic myelogenous (also called chronic myeloid leukemia); Leukemia, hairy cell; Lip and Oral Cavity Cancer; Liver Cancer (Primary); Lung Cancer, Non-Small Cell; Lung Cancer, Small Cell; Lymphomas; Lymphoma, AIDS-related; Lymphoma, Burkitt; Lymphoma, cutaneous T-Cell; Lymphoma, -Hodgkin; Lymphomas, Non-Hodgkin (an old classification of all lymphomas except Hodgkin's); Lymphoma, Primary Central Nervous System; Marcus Whittle, Deadly Disease; Waldenstrom Macroglobulinemia; Malignant Fibrous Histiocytoma of Bone/Osteosarcoma; Medulloblastoma, Childhood: Melanoma; Melanoma, Intraocular (Eye); Merkel Cell Carcinoma: Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myelodysplastic Syndromes: Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer: Oropharyngeal cancer; Osteosarcoma/malignant fibrous histiocytoma of bone; Ovarian cancer; Ovarian epithelial cancer (Surface epithelial-stroma! tumor); Ovarian germ cell tumor; Ovarian tumor malignant potential tumor; Pancreatic cancer: Pancreatic cancer, islet cell; Paranasal sinus and nasal cavity cancer; Parathyroid cancer; Penile cancer; Pharyngeal cancer; Pheochromocytoma; Pineal astrocytoma; Pineal genninoma: Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer: Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sezary syndrome; Skin cancer (nonmelanoma); Skin cancer (melanoma); Skin carcinoma, Merkel cell: Small cell lung cancer; Small intestine cancer: Soft tissue sarcoma; Squamous cell carcinoma—see Skin cancer (nonmelanoma): Squamous neck cancer with occult primary, metastatic; Stomach cancer; Supratentorial primitive neuroectodennal tumor, childhood; T-Cell lymphoma, cutaneous (Mycosis Fungoides and Sezary syndrome); Testicular cancer; Throat cancer; Thymoma, childhood: Thymoma and Thymic carcinoma: Thyroid cancer; Thyroid cancer, childhood; Transitional cell cancer of the renal pelvis and ureter; Trophoblastic tumor, gestational; Unknown primary site, carcinoma of, adult Unknown primary site, cancer of, childhood: Ureter and renal pelvis, transitional cell cancer; Urethral cancer: Uterine cancer, endometrial; Uterine sarcoma; Vaginal cancer: Visual pathway and hypothalarnic glioma, childhood; Vulvar cancer; and Wilms' tumor (kidney cancer)

In some aspects, the tumor antigen is related to a cancer selected from the group consisting of acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, breast cancer, lung cancer, ovarian cancer, prostate cancer, pancreatic cancer, colon cancer, renal cell carcinoma (RCC), and germ cell tumors.

As disclosed herein is a pharmaceutical composition comprising the CyCMV vectors disclosed herein. As disclosed herein is a immunogenic composition comprising the CyCMV vectors disclosed herein. In some aspects, the CyCMV vector is used in the manufacture of a medicament for use in generating an immune response to a pathogen in a subject. In some aspects, the CyCMV vector is used in generating an immune response in a subject.

Also disclosed herein are methods of generating an immune response in a subject. In some aspects, the method comprises administering the CyCMV vector disclosed herein in an amount effective to elicit a CD8+ and/or CD4+ T cell response. In some aspects, the CyCMV vector is used in the manufacture of a medicament for use in generating an immune response in a subject. In some aspects, the CyCMV vector is used in generating an immune response to a pathogen in a subject.

Also disclosed herein are methods of generating an immune response in a subject to a pathogenic infection. In some aspects, the method comprises administering the CyCMV vector disclosed herein in an amount effective to elicit a CD8+ and/or CD4+ T cell response to a pathogen. In some aspects, the CyCMV vector is used in the manufacture of a medicament for use in treating or preventing a pathogenic infection in a subject. In some aspects, the CyCMV vector is used in treating or preventing a pathogenic infection in a subject.

In some aspects, the pathogen is a pathogen selected from the group consisting of: human immunodeficiency virus, simian immunodeficiency virus, herpes simplex virus type 1, herpes simplex virus type 2, hepatitis B virus, hepatitis C virus, papillomavirus, Plasmodium parasites, and Mycobacterium tuberculosis.

Also disclosed herein are methods of treating a tumor in a subject. In some aspects, the method comprises administering the CyCMV vector disclosed herein in an amount effective to elicit a CD8+ and/or CD4+ T cell response to a tumor. In some aspects, the CyCMV vector is used in the manufacture of a medicament for use in treating a tumor in a subject. In some aspects, the CyCMV vector is used in treating a tumor in a subject.

In some aspects, the tumor is related to a cancer selected from the group consisting of: acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, breast cancer, lung cancer, ovarian cancer, prostate cancer, pancreatic cancer, colon cancer, renal cell carcinoma (RCC), and germ cell tumors.

In some aspects, at least 10% of the CD8+ and/or CD4+ T cells elicited by the recombinant CyCMV vector are restricted by MHC-E or an ortholog thereof. In some aspects, at least 20%, at least 30%, at least 40%, at least 50%, at least 60%, at least 70%, at least 75%, at least 80%, at least 85%, at least 90%, or at least 95% of the CD8+ and/or CD4+ T cells elicited by the recombinant CyCMV vector are restricted by MHC-E or an ortholog thereof.

In some aspects, at least 10% of the CD8+ and/or CD4+ T cells elicited by the recombinant CyCMV vector are restricted by MHC-II or an ortholog thereof. In some aspects, at least 20%, at least 30%, at least 40%, at least 50%, at least 60%, at least 70%, at least 75%, at least 80%, at least 85%, at least 90%, or at least 95% of the CD8+ and/or CD4+ T cells elicited by the recombinant CyCMV vector are restricted by MHC-II or an ortholog thereof.

In some aspects, fewer than 10%, fewer than 20%, fewer than 30%, fewer than 40%, or fewer than 50% of the CD8+ and/or CD4+ T cells elicited by the recombinant CyCMV vector are restricted by MHC-I or an ortholog thereof. In some aspects, fewer than 10% of the CD8+ and/or CD4+ T cells elicited by the recombinant CyCMV vector are restricted by MHC-I or an ortholog thereof.

In some aspects, the subject is a human or non-human primate. In some aspects, administering the CyCMV vector comprises subcutaneous, intravenous, intramuscular, intraperitoneal, or oral administration of the CyCMV vector. In some aspects, the method further comprises identifying a TCR from the CD8+ and/or CD4+ T cells elicited by the CyCMV vector, wherein the TCR recognizes a MHC-E/heterologous antigen-derived peptide complex. In some aspects, the method further comprises identifying a TCR from the CD8+ and/or CD4+ T cells elicited by the CyCMV vector, wherein the TCR recognizes a MHC-II/heterologous antigen-derived peptide complex. In some aspects, the TCR is identified by DNA or RNA sequencing.

Also disclosed herein are methods of generating CD8+ and/or CD4+ T cells that recognize MHC-E-peptide complexes, the method comprising: (1) administering to a first subject the CyCMV vector disclosed herein in an amount effective to generate a set of CD8+ and/or CD4+ T cells that recognize MHC-E/peptide complexes; (2) identifying a first TCR from the set of CD8+ and/or CD4+ T cells, wherein the first TCR recognizes a MHC-E/heterologous antigen-derived peptide complex; (3) isolating one or more CD8+ and/or CD4+ T cells from a second subject; and (4) transfecting the one or more CD8+ and/or CD4+ T cells with an expression vector, wherein the expression vector comprises a nucleic acid sequence encoding a second TCR and a promoter operably linked to the nucleic acid sequence encoding the second TCR, wherein the second TCR comprises CDR3α and CDR3β of the first TCR, thereby generating one or more transfected CD8+ and/or CD4+ T cells that recognize a MHC-E/heterologous antigen-derived peptide complex.

In some aspects, the first TCR is identified by DNA or RNA sequencing. In some aspects, the second TCR comprises CDR1α, CDR2α, CDR3α, CDR1β, CDR2β, and CDR3 of the first TCR. In some aspects, the nucleic acid sequence encoding the second TCR is identical to the nucleic acid sequence encoding the first TCR. In some aspects, the method comprises administering the CyCMV vector to the subject comprises intravenous, intramuscular, intraperitoneal, or oral administration of the CyCMV vector to the subject. In some aspects, the subject is a human or nonhuman primate. In some aspects, the method further comprises administering the transfected CD8+ and/or CD4+ T cells to the subject to treat a pathogenic infection.

In some aspects, the at least one heterologous antigen comprises a pathogen-specific antigen, a tumor antigen, a tissue-specific antigen, or a host self-antigen.

In some aspects, the pathogen specific antigen is derived from a pathogen selected from the group consisting of human immunodeficiency virus, simian immunodeficiency virus, herpes simplex virus type 1, herpes simplex virus type 2, hepatitis B virus, hepatitis C virus, papillomavirus, Plasmodium parasites, and Mycobacterium tuberculosis.

In some aspects, the tumor antigen is related to a cancer selected from the group consisting of acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, breast cancer, lung cancer, ovarian cancer, prostate cancer, pancreatic cancer, colon cancer, renal cell carcinoma (RCC), and germ cell tumors.

Also disclosed herein are methods of generating CD8+ and/or CD4+ T cells that recognize MHC-E-peptide complexes, the method comprising: (1) administering to a first subject the CyCMV vector disclosed herein in an amount effective to generate a set of CD8+ and/or CD4+ T cells that recognize MHC-E/peptide complexes; (2) identifying a first TCR from the set of CD8+ and/or CD4+ T cells, wherein the first TCR recognizes a MHC-E/heterologous antigen-derived peptide complex; (3) isolating one or more CD8+ and/or CD4+ T cells from a second subject; and (4) transfecting the one or more CD8+ and/or CD4+ T cells with an expression vector, wherein the expression vector comprises a nucleic acid sequence encoding a second TCR and a promoter operably linked to the nucleic acid sequence encoding the second TCR, wherein the second TCR comprises CDR3α and CDR3β of the first TCR, thereby generating one or more transfected CD8+ and/or CD4+ T cells that recognize a MHC-E/heterologous antigen-derived peptide complex.

In some aspects, the first TCR is identified by DNA or RNA sequencing. In some aspects, the first subject is a human or nonhuman primate. In some aspects, the second subject is a human or nonhuman primate. In some aspects, the first subject is a non-human primate and the second subject is a human and wherein the second TCR is a chimeric non-human primate-human TCR comprising the non-human primate CDR3α and CDR3β of the first TCR. In some aspects, the second TCR comprises the non-human primate CDR1α, CDR2α, CDR3α, CDR1β, CDR2β, and CDR3β of the first TCR. In some aspects, the nucleic acid sequence encoding the second TCR is identical to the nucleic acid sequence encoding the first TCR. In some aspects, the second TCR is a chimeric TCR. In some aspects, the nucleic acid sequence encoding the second TCR is identical to the nucleic acid sequence encoding the first TCR. In some aspects, the method further comprises administering the CyCMV vector to the subject comprises intravenous, intramuscular, intraperitoneal, or oral administration of the CyCMV vector to the subject. In some aspects, the method further comprises administering the transfected CD8+ and/or CD4+ T cells to the subject to treat a pathogenic infection.

In some aspects, the at least one heterologous antigen comprises a pathogen-specific antigen, a tumor antigen, a tissue-specific antigen, or a host self-antigen.

In some aspects, the pathogen specific antigen is derived from a pathogen selected from the group consisting of human immunodeficiency virus, simian immunodeficiency virus, herpes simplex virus type 1, herpes simplex virus type 2, hepatitis B virus, hepatitis C virus, papillomavirus, Plasmodium parasites, and Mycobacterium tuberculosis.

In some aspects, the tumor antigen is related to a cancer selected from the group consisting of acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, breast cancer, lung cancer, ovarian cancer, prostate cancer, pancreatic cancer, colon cancer, renal cell carcinoma (RCC), and germ cell tumors.

Also disclosed herein are methods of generating CD8+ and/or CD4+ T cells that recognize MHC-II-peptide complexes, the method comprising: (1) administering to a subject the CyCMV vector as disclosed herein in an amount effective to generate a set of CD8+ and/or CD4+ T cells that recognize MHC-II/peptide complexes; (2) identifying a first TCR from the set of CD8+ and/or CD4+ T cells, wherein the first TCR recognizes a MHC-II/heterologous antigen-derived peptide complex; (3) isolating one or more CD8+ and/or CD4+ T cells from the subject; and (4) transfecting the one or more CD8+ and/or CD4+ T cells with an expression vector, wherein the expression vector comprises a nucleic acid sequence encoding a second TCR and a promoter operably linked to the nucleic acid sequence encoding the second TCR, wherein the second TCR comprises CDR3α and CDR3β of the first TCR, thereby generating one or more transfected CD8+ and/or CD4+ T cells that recognize a MHC-II/heterologous antigen-derived peptide complex.

In some aspects, the first TCR is identified by DNA or RNA sequencing. In some aspects, the second TCR comprises CDR1α, CDR2α, CDR3α, CDR1β, CDR2β, and CDR3β of the first TCR. In some aspects, the nucleic acid sequence encoding the second TCR is identical to the nucleic acid sequence encoding the first TCR. In some aspects, the method further comprises administering the CyCMV vector to the subject comprises intravenous, intramuscular, intraperitoneal, or oral administration of the CyCMV vector to the subject. In some aspects, the subject is a human or nonhuman primate. In some aspects, the method further comprises administering the transfected CD8+ and/or CD4+ T cells to the subject to treat a pathogenic infection.

In some aspects, the at least one heterologous antigen comprises a pathogen-specific antigen, a tumor antigen, a tissue-specific antigen, or a host self-antigen.

In some aspects, the pathogen specific antigen is derived from a pathogen selected from the group consisting of human immunodeficiency virus, simian immunodeficiency virus, herpes simplex virus type 1, herpes simplex virus type 2, hepatitis B virus, hepatitis C virus, papillomavirus, Plasmodium parasites, and Mycobacterium tuberculosis.

In some aspects, the tumor antigen is related to a cancer selected from the group consisting of acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, breast cancer, lung cancer, ovarian cancer, prostate cancer, pancreatic cancer, colon cancer, renal cell carcinoma (RCC), and germ cell tumors.

Also disclosed herein are methods of generating CD8+ and/or CD4+ T cells that recognize MHC-II-peptide complexes, the method comprising: (1) administering to a first subject the CyCMV vector as disclosed herein in an amount effective to generate a set of CD8+ and/or CD4+ T cells that recognize MHC-II/peptide complexes; (2) identifying a first TCR from the set of CD8+ and/or CD4+ T cells, wherein the first TCR recognizes a MHC-II/heterologous antigen-derived peptide complex; (3) isolating one or more CD8+ and/or CD4+ T cells from a second subject; and (4) transfecting the one or more CD8+ and/or CD4+ T cells with an expression vector, wherein the expression vector comprises a nucleic acid sequence encoding a second TCR and a promoter operably linked to the nucleic acid sequence encoding the second TCR, wherein the second TCR comprises CDR3α and CDR3β of the first TCR, thereby generating one or more transfected CD8+ and/or CD4+ T cells that recognize a MHC-II/heterologous antigen-derived peptide complex.

In some aspects, the first TCR is identified by DNA or RNA sequencing. In some aspects, the first subject is a non-human primate and the second subject is a human and wherein the second TCR is a chimeric non-human primate-human TCR comprising the non-human primate CDR3α and CDR3 of the first TCR. In some aspects, the second TCR comprises the non-human primate CDR1α, CDR2α, CDR3α, CDR1β, CDR2β, and CDR3β of the first TCR. In some aspects, the nucleic acid sequence encoding the second TCR is identical to the nucleic acid sequence encoding the first TCR. In some aspects, the second TCR is a chimeric TCR. In some aspects, the nucleic acid sequence encoding the second TCR is identical to the nucleic acid sequence encoding the first TCR. In some aspects, the method comprises administering the CyCMV vector to the subject comprises intravenous, intramuscular, intraperitoneal, or oral administration of the CyCMV vector to the subject. In some aspects, the method comprises administering the transfected CD8+ and/or CD4+ T cells to the subject to treat a pathogenic infection.

In some aspects, the at least one heterologous antigen comprises a pathogen-specific antigen, a tumor antigen, a tissue-specific antigen, or a host self-antigen.

In some aspects, the pathogen specific antigen is derived from a pathogen selected from the group consisting of human immunodeficiency virus, simian immunodeficiency virus, herpes simplex virus type 1, herpes simplex virus type 2, hepatitis B virus, hepatitis C virus, papillomavirus, Plasmodium parasites, and Mycobacterium tuberculosis.

In some aspects, the tumor antigen is related to a cancer selected from the group consisting of acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, breast cancer, lung cancer, ovarian cancer, prostate cancer, pancreatic cancer, colon cancer, renal cell carcinoma (RCC), and germ cell tumors.

### EXAMPLES

### Example 1: Generation of a 68-1 Like CyCMV Vector

CyCMV was isolated from the urine of healthy Mauritian-origin cynomolgus macaque (MCM) with the goal to recapitulate in a different non-human primate species the previously reported ability of strain 68-1-derived rhesus CMV recombinants to be genetically programmed to elicit MHC-I, MHC-II or MHC-E-restricted CD8+ T cells. MCM, an insular macaque species with reduced genetic complexity, are the ideal species to study MHC-restricted CD8+ T cell responses due to similar immunogenetics as humans in the MHC locus and conserved function between HLA-E, HLA-I and HLA-II in humans and Mafa-E, Mafa-I and Mafa-II in MCM. To acquire full-length CyCMV isolates with as little in vitro adaptation as possible, passaging of the virus was kept to an absolute minimum, with no more than 4 passages of in vitro culture prior to sequencing analysis and subsequent BAC capture. Indeed, a BAC clone containing full length CyCMV with minimal in vitro adaptation for subsequent genetic manipulation was successfully generated.

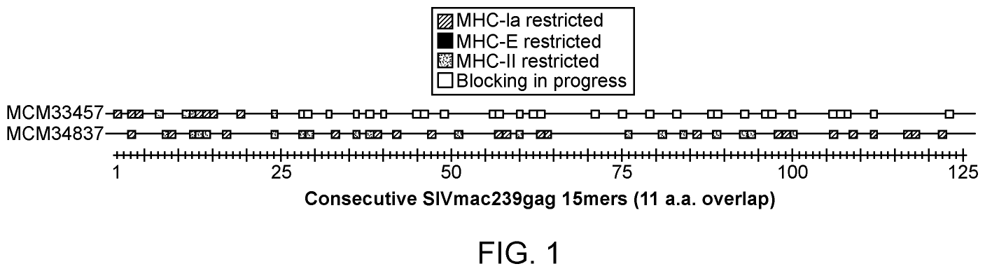

The absence of UL 128 and UL 130 genes was previously identified as being critical for the induction of MHC-E and MHC-II-restricted CD8+ T cells in RM (Hansen, S. G. et al. Broadly targeted CD8 T cell responses restricted by major histocompatibility complex E. Science 351, 714-720 (2016)). Thus, both UL128 and UL130 were deleted from CyCMV and SIVmac239 Gag was inserted to make a ΔUL128, ΔUL130 CyCMV/Gag vaccine vector. Two MCM were vaccinated and the individual CD8+ T cell responses were mapped against Gag and their MHC restriction was defined via the previously described MHC blocking reagents for MHC-I (W6/32 antibody), MHC-II (anti-MHC-II antibody) or MHC-E (VL9 peptide) (Hansen, S. G. et al. Broadly targeted CD8 T cell responses restricted by major histocompatibility complex E. Science 351, 714-720 (2016)). The presence of MHC-II- and MHC-Ia-restricted CD8+ T cells were identified, but no MHC-E-restricted CD8+ T cells were identified in ΔUL128, ΔUL130 CyCMV/Gag vaccinated MCM (FIG. 1). These results indicated that additional viral factors were absent in strain 68-1 RhCMV, which must also be removed from ΔUL128, ΔUL 130 CyCMV to fully recapitulate RhCMV strain 68-1.

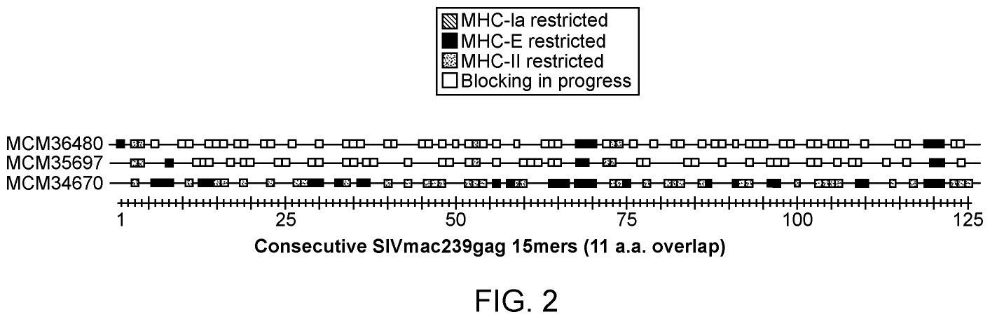

It was identified that UL146 genes, viral proteins with alpha chemokine function (Oxford, K. L. et al. Protein coding content of the UL-b′ region of wild-type rhesus cytomegalovirus. Virology 373, 181-188 (2008); Lüttichau, H. R. The cytomegalovirus UL 146 gene product vCXCLI targets both CXCR1 and CXCR2 as an agonist. J Biol Chem 285, 9137-9146 (2010)), were also deleted by the original genomic inversion that removed UL128 and UL 130 in RhCMV 68-1. It was further shown that the combined deletion of homologs of UL128, UL 130 and all six UL146 homologs from a complete RhCMV genome was required to elicit MHC-E restricted CD8+ T cells. Thus, all six UL 146 homologs were deleted from ΔUL128, ΔUL 130 CyCMV/Gag to generate a “68-1 RhCMV-like” CyCMV/Gag (CyCMV double deleted (dd)). Vaccination of MCM with dd-CyCMV/Gag resulted in both MHC-II- and MHC-E-restricted CD8+ T cells, with broad epitope targeting similar to 68-1 RhCMV/Gag-vaccinated RM (FIG. 2). Remarkably, in these two dd-CyCMV/Gag-vaccinated MCM, targeting of the same MHC-II- and MHC-E-restricted “supertopes”, Gag epitopes targeted by CD8+ T cells in every strain 68-1 RhCMV/Gag vaccinated RM, indicating that dd-CyCMV functions analogously in MCM as strain 68-1 RhCMV does in RM.

### Example 2: Generation of Dissemination Impaired 68-1 Like CyCMV Vectors

With potential clinical applications in mind, and to match clinical vectors that are currently in phase I studies, additional CyCMV vectors were prepared with deletion of the CMV pp71 gene (Cy110) to limiting vivo dissemination and spreading of both FL CyCMV and dd CyCMV vaccine vectors following vaccination and thus unable to be shed from the vaccine recipient.

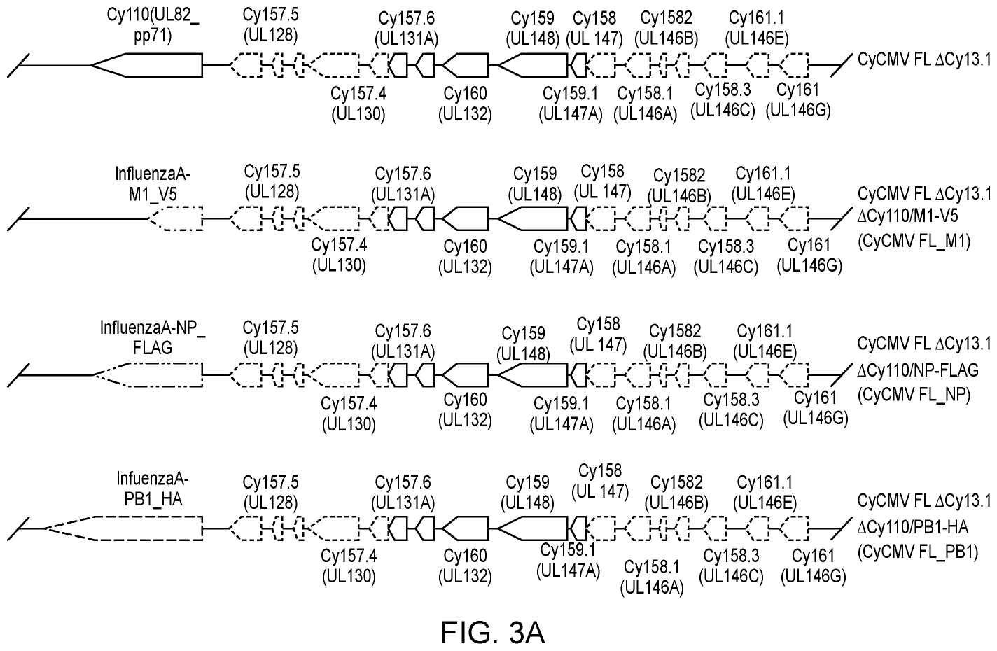

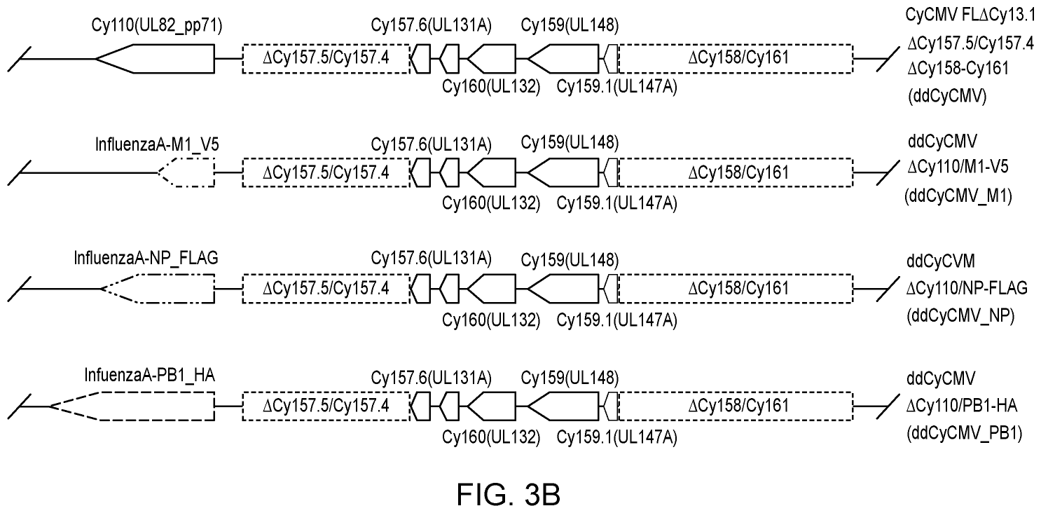

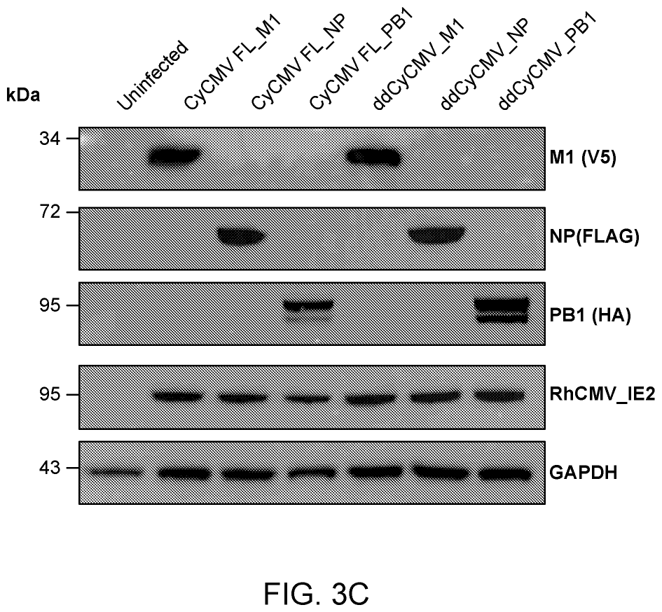

Additionally, three separate vaccine constructs were made by inserting the coding sequence for the 1918 influenza matrix (M), nucleoprotein (NP), and polymerase basic 1 (PB-1) proteins under the Cy110 endogenous promoter in either FL CyCMV or dd CyCMV, yielding two sets of vaccine vectors expressing influenza antigens (FIGS. 3A-3C). These vaccine vector sets were termed FL CyCMVΔCy 110/Flu and dd CyCMVΔCy110/Flu, respectively. This change in challenge strain offered the ability to more stringently test the ability of the vaccine approach to tolerate sequence diversity as this heterologous challenge virus is separated from the 1918 influenza sequences that were used as antigens by approximately 90 years of natural influenza sequence evolution.

### Example 3: Immunology of MCM Vaccinated with Dissemination-Impaired CyCMV Vectors Expressing 1918 Influenza Antigens

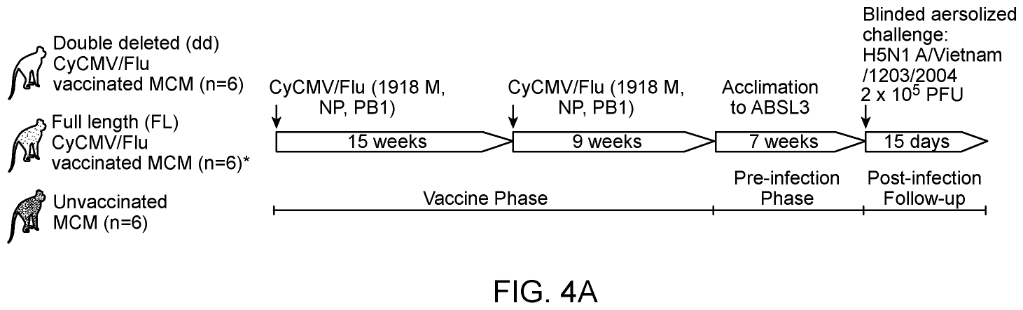

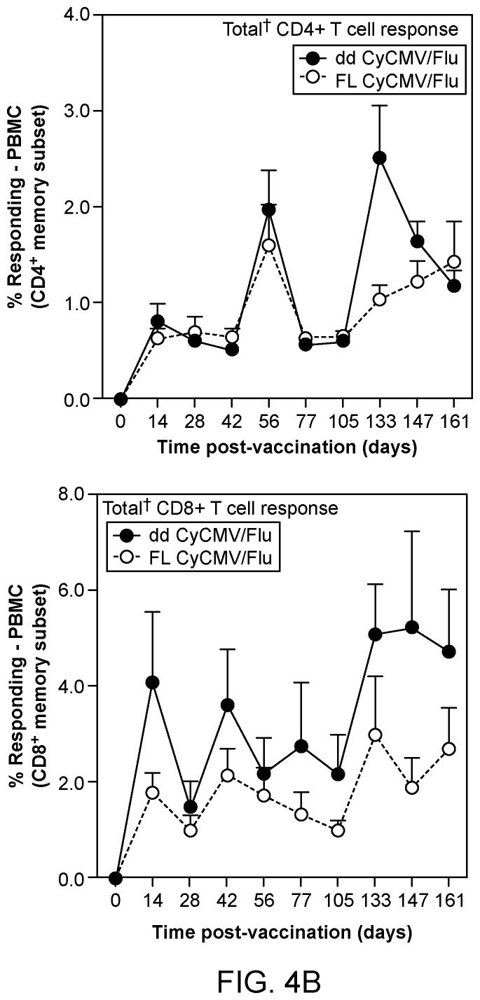

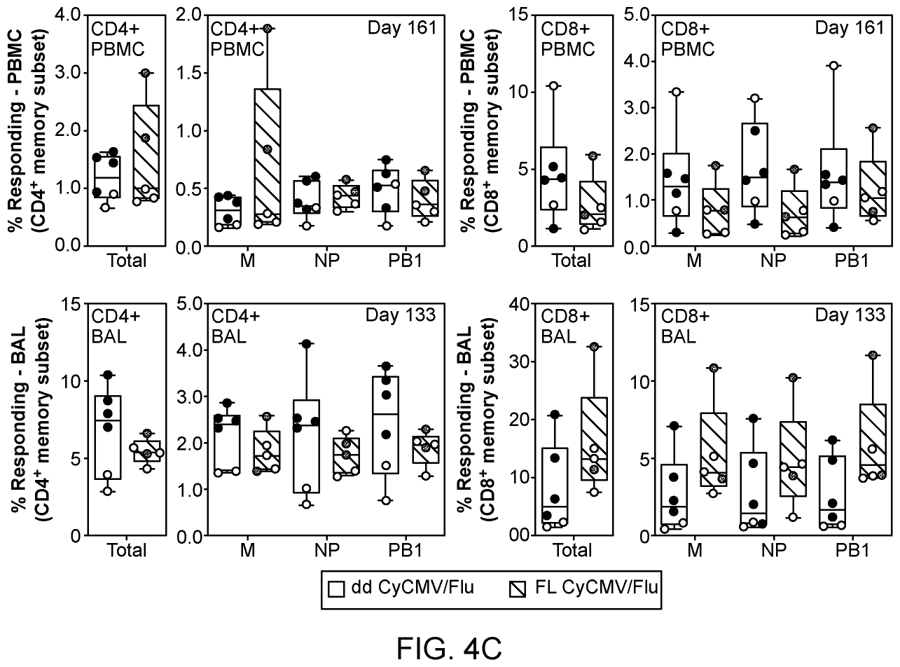

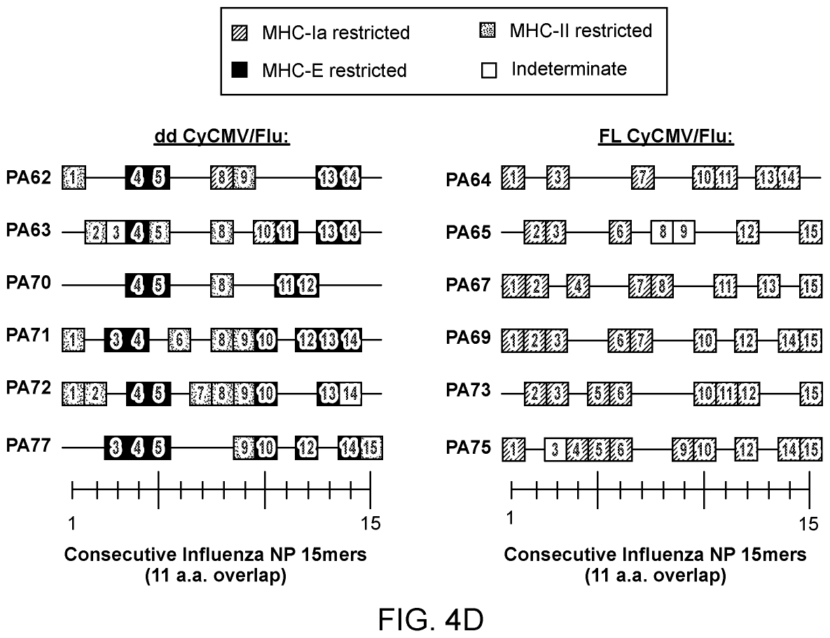

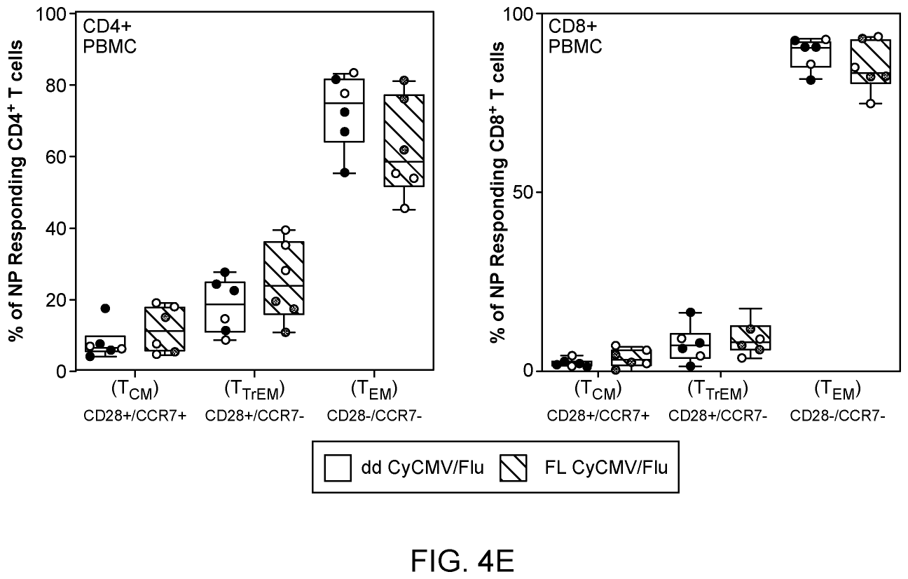

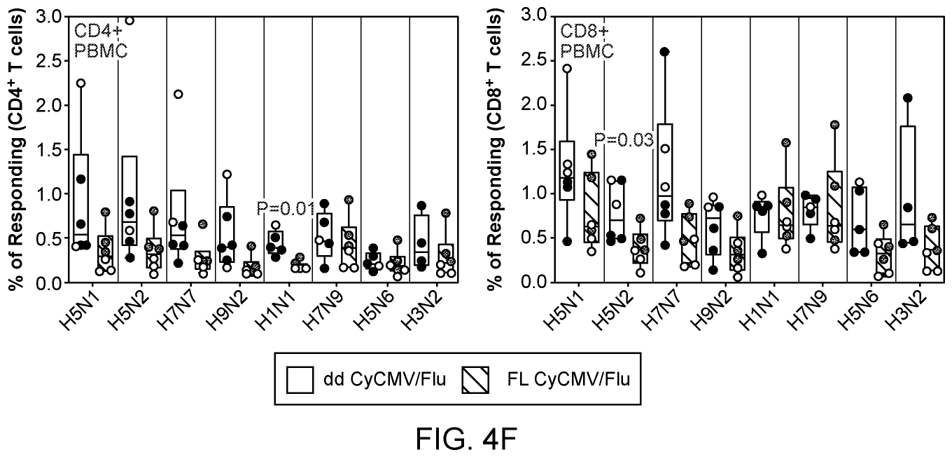

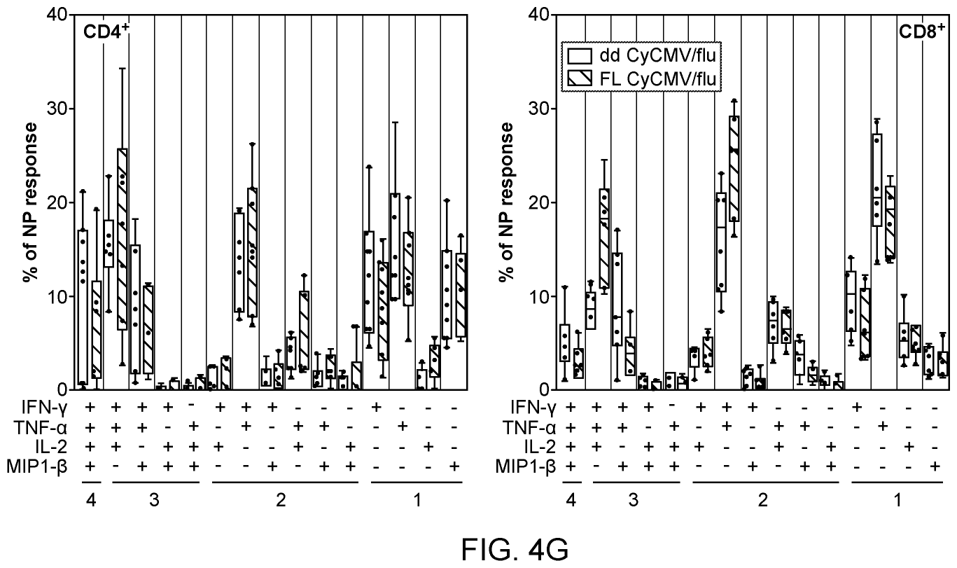

Six MCM were vaccinated subcutaneously with 1×10⁷ PFU each of the FL CyCMV/ΔCy110Flu or dd CyCMVΔCy110/Flu, and boosted at 16 weeks post-prime (FIG. 4A). Unfortunately, one FL CyCMVΔCy110/Flu MCM died from study-unrelated causes approximately one month post boost, limiting downstream analyses. Influenza transgene-specific CD4+ and CD8+ T cells in blood were monitored, and influenza-specific responses were detectable throughout the vaccine induction phase with no significant difference observed in the magnitude of CD4+ or CD8+ T cell responses between FL CyCMVΔCy 110/Flu or dd CyCMVΔCy110/Flu vaccinated MCM (FIG. 4B). These influenza-specific T cell responses were maintained until the MCM underwent influenza challenge. Importantly, robust influenza-specific pulmonary T cell responses were observed in the lung via assessing T cells found in bronchoalveolar lavage (BAL) at the final pre-challenge time point (FIG. 4C).

To understand the MHC restriction of the CD8+ T cell responses induced by FL CyCMVΔCy110/Flu or dd CyCMVΔCy110/Flu vectors, the specificity of NP-specific CD8+ T cells was assessed by stimulating peripheral blood mononuclear cells (PBMC) with 15-mer peptides that span the first 71 amino acids of the NP open reading frame in an intracellular cytokine staining (ICS) assay. The MHC restriction of all peptides capable of inducing a CD8+ T cell response was subsequently defined by performing the above assay in the presence of each of the following: the pan MHC-I-blocking antibody W6/32, the leader sequence-derived MHC-E-blocking VL9 peptide, the MHC-II-blocking G46.6 antibody, or isotype control reagents.

As expected, the FL CyCMVΔCy110/NP vector engendered conventionally MHC-Ia-restricted CD8+ T cells, while dd CyCMVΔCy110/NP vector engendered unconventionally MHC-E- and MHC-II-restricted CD8+ T cells (FIG. 4D). Consistent with the previous observations of dd CyCMV/SIV-vaccinated MCM mounting SIV transgene-specific CD8+ T cells displaying an effector memory (EM) phenotype, both FL and dd CyCMVΔCy110/Flu vectors induced influenza-transgene-specific CD4+ and CD8+ T cells that were predominantly EM in phenotype (FIG. 4E).

Finally, to measure the ability of FL and dd CyCMVΔCy 110/Flu-induced CD4+ and CD8+ T cells to recognize variation within the M, NP, an PB1 proteins present within circulating influenza strains, a recognition assay was performed by co-culturing PBMC from FL and dd CyCMVΔCy110/Flu-vaccinated MCM with eight different inactivated primary influenza isolates (H5N1, H5N2, H7N7, H9N2, H1N1, H7N9, H5N6, and H3N2) and measuring T cell activation via ICS (FIG. 4F). While the magnitude of T cell recognition varied across the eight primary isolates tested, all eight isolates were robustly recognized by both CD4+ and CD8+ T cells from both FL and dd CyCMVΔCy110/Flu-vaccinated MCM. Therefore, these results supported the hypothesis that targeting conserved internal influenza proteins represents a viable pathway towards universal influenza vaccine design. The polyfunctionality profile of CD4+ (FIG. 4G, left) or CD8+ (FIG. 4G, right) T cells targeting the NP insert in either FL or dd CyCMV was assessed.

### Example 4: Aerosolized H5N1 Challenge of Dissemination-Impaired CyCMV/Influenza-Vaccinated and Unvaccinated MCM

Following conclusion of the vaccine immunogenicity phase of the experiment, all vaccinated MCM, along with six unvaccinated MCM, were shipped to the University of Pittsburgh for challenge with aerosolized avian H5N1 influenza (FIG. 4A). This virus strain and route of challenge was selected due to more relevant route of challenge, and documented ability to induce severe disease with uniform lethality (Wonderlich, E. R. et al. Widespread Virus Replication in Alveoli Drives Acute Respiratory Distress Syndrome in Aerosolized H5N1 Influenza Infection of Macaques. J Immuno/198, 1616-1626 (2017)). Importantly, to avoid introducing bias into the challenge experiment, the team at the University of Pittsburgh were blinded to the vaccination status of all MCM (FIG. 4A).

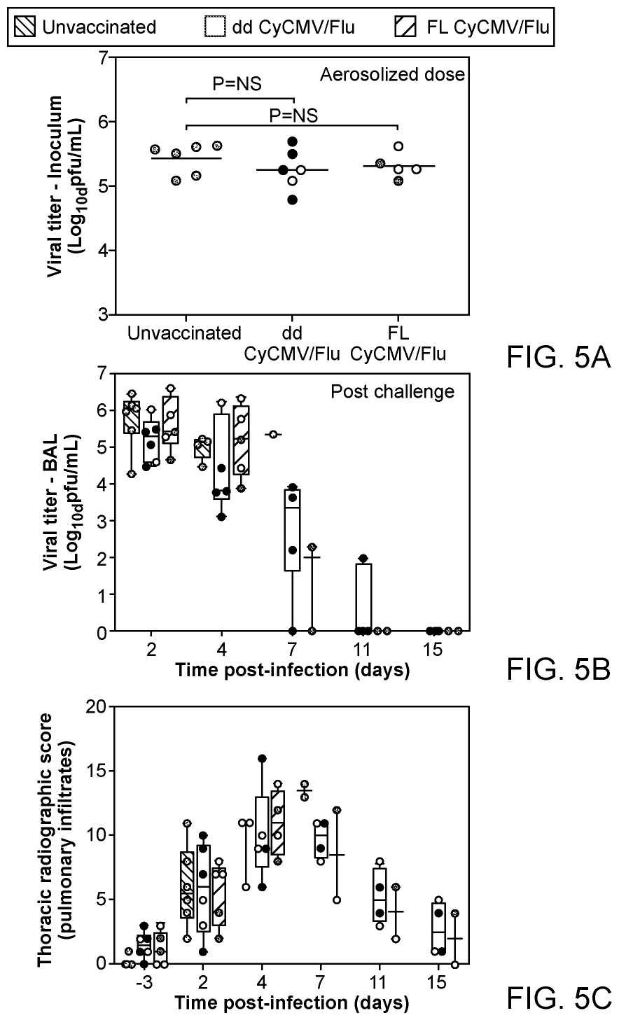

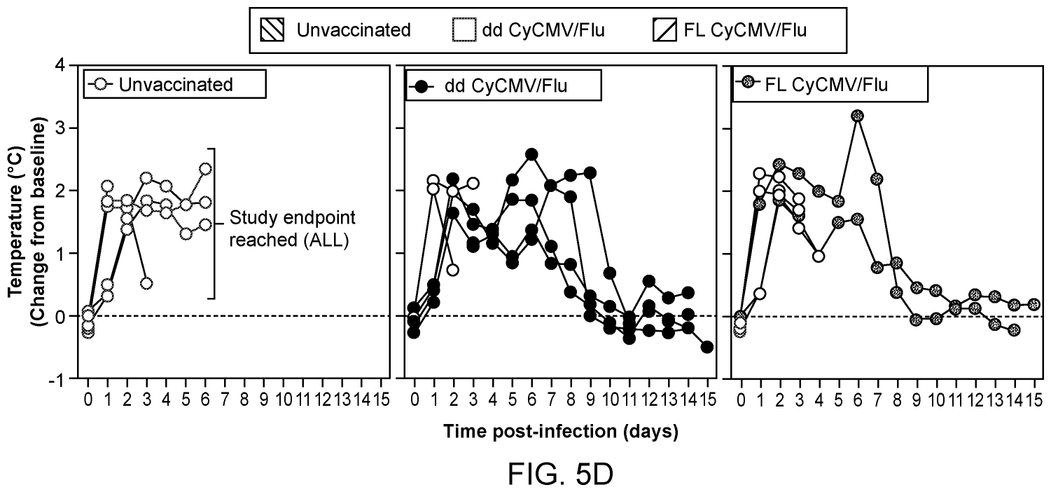

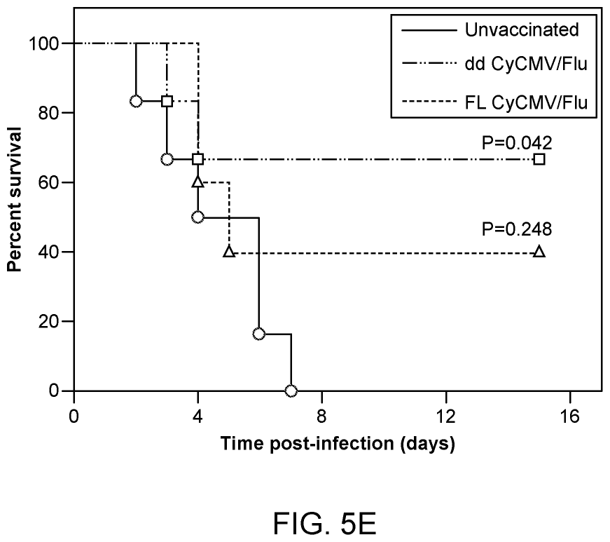

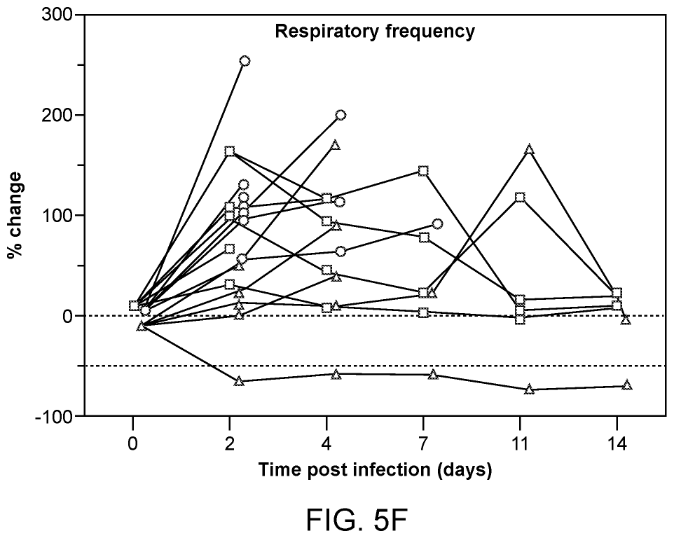

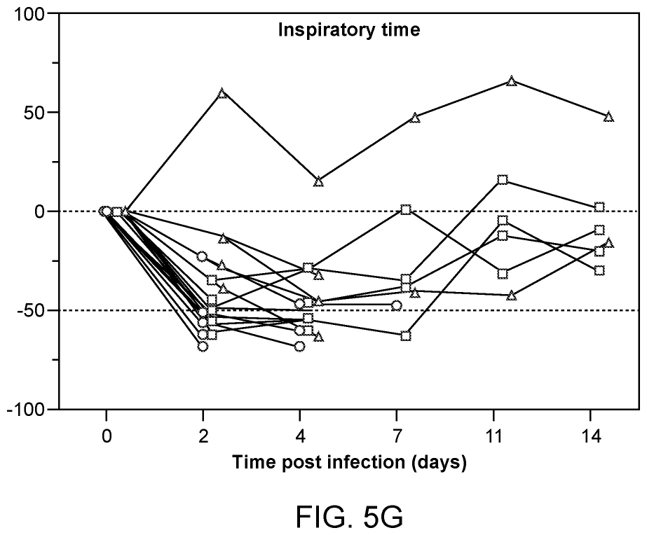

All 17 MCM underwent challenge with small-particle aerosols of the highly pathogenic avian influenza isolate A/Vietnam/1203/2004 (H5N1), with no significant differences measured in the challenge dose inhaled between the three groups (FIG. 5A). All MCM became infected with robust viral replication observed in bronchoalveolar lavage (BAL) of all animals beginning two days after challenge (FIG. 5B). No statistically significant difference was found between the amount of virus in the BAL between the three groups. Thoracic radiographs, scored by a radiologist blinded to the treatment of all animals, revealed inflammation uniformly across all challenged MCM, but again with no statistically significant difference between the three groups (FIG. 5C). All MCM developed a fever following infection, which subsequently resolved in approximately half of the vaccinated MCM, but never resolved in unvaccinated controls (FIG. 5D). In line with the unresolved fever observed in unvaccinated controls, all six of these MCM meeting pre-established criteria for euthanasia by day seven post infection (FIG. 5E). In contrast, CyCMV/Flu-vaccination significantly protected MCM recipients as these MCM resolved infection and survived, while two out of five FL CyCMV/Flu-vaccinated MCM survived (FIG. 5E). Thus, while CMV vector-based vaccination did not prevent infection or pulmonary inflammation, it did protect against death from influenza infection. No Changes in respiratory frequency (FIG. 5F) and inspiratory time (FIG. 5G) were noted between the groups.

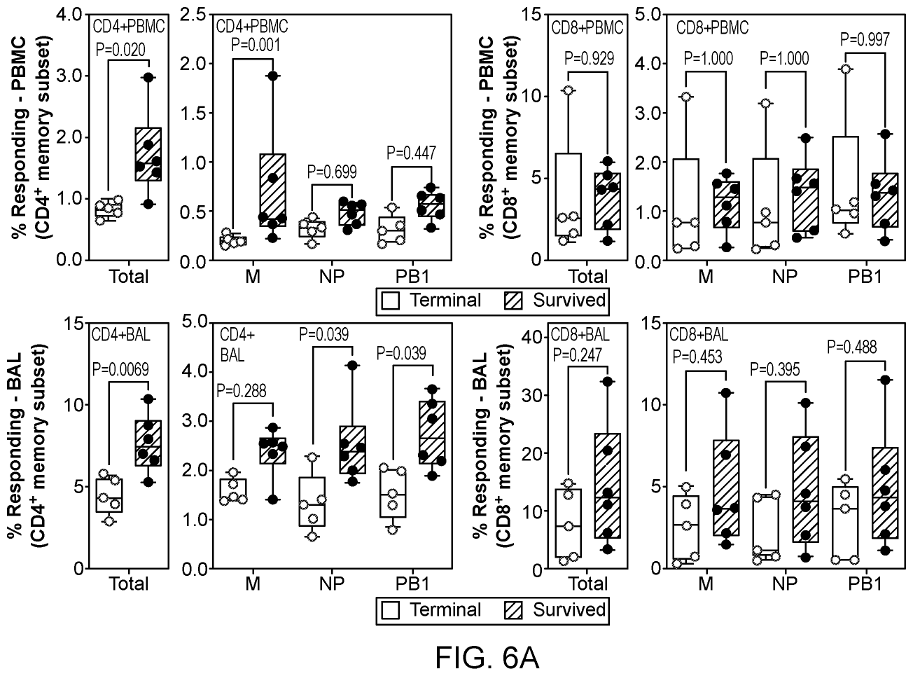

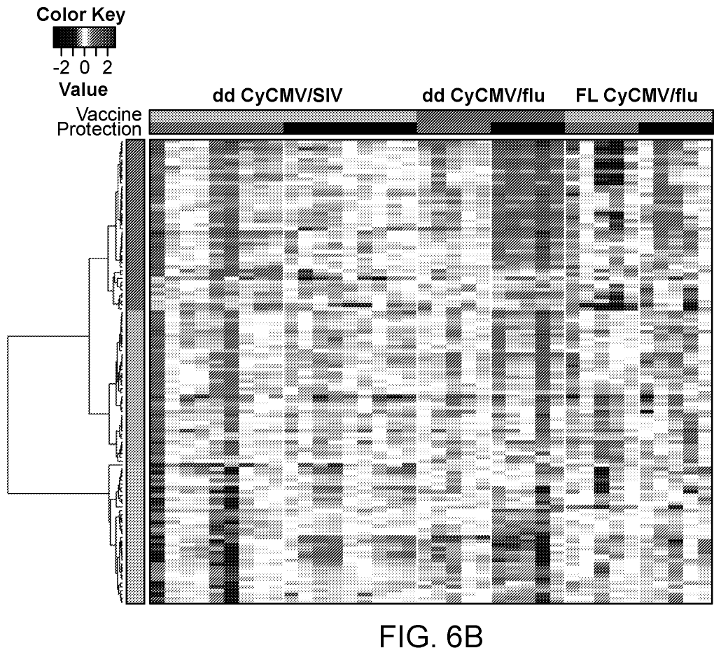

To determine what CyCMV vector-induced immune responses might correlate with protection from death following aerosolized H5N1 infection, all vaccinated MCM were combined into two groups based on survival outcome, regardless of the vaccine vector received, and their influenza transgene-specific T cell responses were analyzed immediately prior to challenge. This analysis revealed that the magnitude of the total influenza transgene-specific CD4+ T cell response in peripheral blood prior to challenge significantly correlated with protection from death (FIG. 6A). In contrast, the magnitude of the CD8+ T cell response was not correlated with protection. When the individual antigen-specific T cell responses were analyzed, the M-specific CD4+ T cell response correlated significantly with protection, while no other individual antigen-specific response did. When the same analysis was performed with the final BAL sample before challenge to examine the response in the lung, the magnitude of the total influenza transgene-specific CD4+ T cell response again correlated with protection while the CD8+ T cell response did not (FIG. 6A). In the BAL, the magnitude of both NP- and PB1-specific CD4+ T cells correlated significantly with protection, while no CD8+ T cell response did. Therefore, this analysis revealed the magnitude of the CD4+ T cell response in both blood and the lung as predictors of vaccine efficacy.

The loss of one FL CyCMV/Flu-vaccinated MCM during the vaccine phase precluded any assessment of differences in protection engendered by the two vaccine vectors in terms of impact of the MHC-restriction profile of the CD8+ T cells. While it remains possible that differences in the MHC restriction of the influenza-specific CD8+ T cells influenced, the observation above indicated that CD4+ T cells, and not CD8+ T cells, regardless of their MHC restriction, were critical mediators of protection from death following influenza infection. It has been previously demonstrated that MHC-E-restricted CD8+ T cells are critical mediators of the protection observed against SIV replication in RhCMV/SIV-vaccinated rhesus macaques (Malouli, D. et al. Cytomegaloviral determinants of CD8″ T cell programming and RhCMV/SIV vaccine efficacy. Sci Immunol 6, (2021)). Furthermore, it has been demonstrated that the MHC-E-restricted CD8+ T cell-mediated protection against SIV replication is accompanied by a strong IL-15-focused transcriptomic signature (Malouli, D. et al. Cytomegalovirus-vaccine-induced unconventional T cell priming and control of SIV replication is conserved between primate species. Cell Host Microbe 30, 1207-1218.e7 (2022)); (Barrenäs, F. et al. Interleukin-15 response signature predicts RhCMV/SIV vaccine efficacy. PLoS Pathog 17, e1009278 (2021)). Therefore, IL-15 whole blood transcriptomics analysis of CyCMV/Flu-vaccinated MCM was performed and found that it was absent from CyCMV/Flu-protected MCM (FIG. 6B). This finding further supports the finding that CyCMV/Flu-mediated protection from death following influenza challenge does not depend on CD8+ T cells, but rather on CD4+ T cells that are induced by both FL and dd CyCMV vectors.

Cumulatively, the results presented here provide solid proof-of-concept data that CMV-vectored vaccine vectors are able to elicit CD4+ and CD8+ T cells capable of protecting against lethal influenza challenge. Of note, approximately 90 years of natural influenza evolution separate the vaccine and challenge sequences utilized in this study, showing this cellular immune response-based vaccine approach may overcome the wide range of sequence variation that exist in seasonal and pandemic influenza. Although it was initially hypothesized that protection would depend on CD8+ T cells, the results support the converse. Instead, vaccine-induced CD4+ T cells emerged as the likely mediators of the protection observed.

What is claimed is:

1. A recombinant cytomegalovirus (CMV) vector comprising:
(a) a first nucleic acid sequence encoding an influenza matrix protein;
(b) a second nucleic acid sequence encoding an influenza nucleoprotein; and
(c) a third nucleic acid sequence encoding an influenza polymerase.

2. The CMV vector of claim 1, wherein the vector does not express an active UL146 protein or ortholog thereof and does not express an active UL147 protein or ortholog thereof.

3. A recombinant cytomegalovirus (CMV) vector comprising:
(a) a first nucleic acid sequence encoding an influenza matrix; and/or
(b) a second nucleic acid sequence encoding an influenza nucleoprotein; and/or
(c) a third nucleic acid sequence encoding an influenza polymerase;
wherein the vector does not express an active UL128 protein or ortholog thereof; does not express an active UL130 protein or ortholog thereof; does not express an active UL 146 protein or ortholog thereof; does not express an active UL 147 protein or ortholog thereof; and does not express an active UL18 protein or ortholog thereof.

4. The CMV vector of claim 2, wherein the CMV vector comprises (a) a first nucleic acid sequence encoding an influenza matrix; (b) a second nucleic acid sequence encoding an influenza nucleoprotein; and (c) a third nucleic acid sequence encoding an influenza polymerase.

5. The CMV vector of any of the preceding claims, wherein the CMV vector does not express an active pp71 protein or ortholog thereof.

6. The CMV vector of any one of the preceding claims, wherein the CMV vector is a human CMV (HCMV) vector, a rhesus macaque CMV (RhCMV) vector, or a cynomolgus macaque CMV (CyCMV) vector.

7. The CMV vector of claim 6, wherein the CMV vector is a cynomolgus macaque CMV (CyCMV) vector.

8. The CMV vector of any of the preceding claims, wherein at least one of the influenza matrix protein, influenza nucleoprotein, or influenza polymerase is derived from a H1N1 influenza virus.

9. The CMV vector of any of the preceding claims, wherein the influenza matrix protein, influenza nucleoprotein, and influenza polymerase are derived from a H1N1 influenza virus.

10. The CMV vector of any of the preceding claims, wherein at least one of the influenza matrix protein, influenza nucleoprotein, or influenza polymerase is derived from a 1918 H1N1 influenza virus.

11. The CMV vector of any of the preceding claims, wherein the influenza matrix protein, influenza nucleoprotein, and influenza polymerase are derived from a 1918 H1N1 influenza virus.

12. The CMV vector of any of the preceding claims, wherein the nucleic acid sequence encoding the matrix protein is a wild-type or a codon-optimized nucleic acid sequence.

13. The CMV vector of claim 12, wherein the nucleic acid sequence encoding the matrix protein is a codon-optimized nucleic acid sequence.

14. The CMV vector of any of the preceding claims, wherein the nucleic acid sequence encoding the matrix protein has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the sequence of SEQ ID NO: 1.

15. The CMV vector of any of the preceding claims, wherein the matrix protein has an amino acid sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the sequence of SEQ ID NO: 10.

16. The CMV vector of any of the preceding claims, wherein the nucleic acid sequence encoding the nucleoprotein is a wild-type or a codon-optimized nucleic acid sequence.

17. The CMV vector of claim 16, wherein the nucleic acid sequence encoding the nucleoprotein is a codon-optimized nucleic acid sequence.

18. The CMV vector of any of the preceding claims, wherein the nucleic acid sequence encoding the nucleoprotein has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the sequence of SEQ ID NO: 3.

19. The CMV vector of any of the preceding claims, wherein the nucleoprotein has an amino acid sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the sequence of SEQ ID NO: 12.

20. The CMV vector of any of the preceding claims, wherein the nucleic acid sequence encoding the polymerase encodes a polymerase basic 1 (PB1) protein, a polymerase basic 2 (PB2) protein, or a polymerase acidic protein.

21. The CMV vector of claim 20, wherein the nucleic acid sequence encoding the polymerase encodes a polymerase basic 1 (PB1) protein.

22. The CMV vector of any one of the preceding claims, wherein the nucleic acid sequence encoding the polymerase is a wild-type or a codon-optimized nucleic acid sequence.

23. The CMV vector of claim 22, wherein the nucleic acid sequence encoding the polymerase is a codon-optimized nucleic acid sequence.

24. The CMV vector of any of the preceding claims, wherein the nucleic acid sequence encoding the polymerase has at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the sequence of SEQ ID NO: 5.

25. The CMV vector of any of the preceding claims, wherein the polymerase has an amino acid sequence with at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to the sequence of SEQ ID NO: 14.

26. The CMV vector of any one of the preceding claims, wherein the nucleic acid sequence encoding the matrix protein, the nucleic acid sequence encoding the nucleoprotein, and the nucleic acid sequence encoding the polymerase are under the transcriptional control of a UL82 promoter, CMV promoter, a CMV-gH promoter, a HCMV promoter, a RhCMV promoter, a EF1α promoter, a CAG promoter, or a MCMV promoter.

27. The CMV vector of claim 26, wherein the matrix, nucleoprotein, and polymerase are under the transcriptional control of a UL82 promoter.

28. A pharmaceutical composition comprising the CMV vector of any of claims 1-27 and a pharmaceutically acceptable carrier.

29. An immunogenic composition comprising the CMV vector of any of claims 1-27 and a pharmaceutically acceptable carrier.

30. A method of generating an immune response in a subject, comprising administering to the subject the CMV vector, pharmaceutical composition, or immunogenic composition of any of claims 1-29 in an amount effective to elicit a CD8+ and/or CD4+ T cell response.

31. Use of the CMV vector, pharmaceutical composition, or immunogenic composition of any of claims 1-29 in the manufacture of a medicament for use in generating an immune response in a subject.

32. The CMV vector, pharmaceutical composition, or immunogenic composition of any of claims 1-29 for use in generating an immune response in a subject.

33. A method of generating an immune response in a subject to a pathogenic infection, comprising administering to the subject the CMV vector, pharmaceutical composition, or immunogenic composition of any of claims 1-29 in an amount effective to elicit a CD8+ and/or CD4+ T cell response to a pathogen.

34. Use of the CMV vector, pharmaceutical composition, or immunogenic composition of any of claims 1-29 in the manufacture of a medicament for use in generating an immune response to a pathogen in a subject.

35. The CMV vector, pharmaceutical composition, or immunogenic composition of any of claims 1-29 for use in generating an immune response to a pathogen in a subject.

36. A method of treating or preventing a pathogenic infection in a subject, comprising administering the CMV vector, pharmaceutical composition, or immunogenic composition of any of claims 1-29 in an amount effective to elicit a CD8+ and/or CD4+ T cell response to the pathogen.

37. Use of the CMV vector, pharmaceutical composition, or immunogenic composition of any of claims 1-29 in the manufacture of a medicament for use in treating or preventing a pathogenic infection in a subject.

38. The CMV vector, pharmaceutical composition, or immunogenic composition of any of claims 1-29 for use in treating or preventing a pathogenic infection in a subject.

39. The method, CMV vector, pharmaceutical composition, or immunogenic composition for use, or use in manufacture of any of claims 1-38, wherein the pathogen is an influenza virus.

40. The method, CMV vector, pharmaceutical composition, or immunogenic composition for use, or use in manufacture of claim 39, wherein the influenza virus is a H1N1 virus, a H5N1 virus, a H2N2 virus, or a H3N2 virus.

41. The method, CMV vector, pharmaceutical composition, or immunogenic composition for use, or use in manufacture of any of claims 1-38, wherein the pathogen is heterologous to the antigens encoded on the CMV vector.

42. The method, CMV vector, pharmaceutical composition, or immunogenic composition for use, or use in manufacture of claim 41, wherein the pathogen is a H5N1 virus, a H2N2 virus, or a H3N2 virus.

43. The method, CMV vector, pharmaceutical composition, or immunogenic composition for use, or use in manufacture of any of claims 1-2 and 4-42, wherein at least 10% of the CD8+ and/or CD4+ T cells elicited by the recombinant CMV vector are restricted by MHC-I or an ortholog thereof.

44. The method, CMV vector, pharmaceutical composition, or immunogenic composition for use, or use in manufacture of claim 43, wherein at least 20%, at least 30%, at least 40%, at least 50%, at least 60%, at least 70%, at least 75%, at least 80%, at least 85%, at least 90%, or at least 95% of the CD8+ and/or CD4+ T cells elicited by the recombinant CMV vector are restricted by MHC-I or an ortholog thereof.

45. The method, CMV vector, pharmaceutical composition, or immunogenic composition for use, or use in manufacture of any of claims 3-42, wherein at least 10% of the CD8+ and/or CD4+ T cells elicited by the recombinant CMV vector are restricted by MHC-E or an ortholog thereof.

46. The method, CMV vector, pharmaceutical composition, or immunogenic composition for use, or use in manufacture of claim 45, wherein at least 20%, at least 30%, at least 40%, at least 50%, at least 60%, at least 70%, at least 75%, at least 80%, at least 85%, at least 90%, or at least 95% of the CD8+ and/or CD4+ T cells elicited by the recombinant CMV vector are restricted by MHC-E or an ortholog thereof.

47. The method, CMV vector, pharmaceutical composition, or immunogenic composition for use, or use in manufacture of any of claims 3-42, wherein at least 10% of the CD8+ and/or CD4+ T cells elicited by the recombinant CMV vector are restricted by MHC-II or an ortholog thereof.

48. The method, CMV vector, pharmaceutical composition, or immunogenic composition for use, or use in manufacture of claim 47, wherein at least 20%, at least 30%, at least 40%, at least 50%, at least 60%, at least 70%, at least 75%, at least 80%, at least 85%, at least 90%, or at least 95% of the CD8+ and/or CD4+ T cells elicited by the recombinant CMV vector are restricted by MHC-II or an ortholog thereof.

49. The method, CMV vector, pharmaceutical composition, or immunogenic composition for use, or use in manufacture of claims 3-42 and 45-48, wherein fewer than 10%, fewer than 20%, fewer than 30%, fewer than 40%, or fewer than 50% of the CD8+ and/or CD4+ T cells elicited by the recombinant CMV vector are restricted by MHC-I or an ortholog thereof.

50. The method, CMV vector, pharmaceutical composition, or immunogenic composition for use, or use in manufacture of claim 49, wherein fewer than 10% of the CD8+ and/or CD4+ T cells elicited by the recombinant CMV vector are restricted by MHC-I or an ortholog thereof.

51. The method, CMV vector, pharmaceutical composition, or immunogenic composition for use, or use in manufacture of any of claims 30-50, wherein the subject is a human or non-human primate.

52. The method, CMV vector, pharmaceutical composition, or immunogenic composition for use, or use in manufacture of any of claims 30-51, wherein administering the CMV vector comprises subcutaneous, intravenous, intramuscular, intraperitoneal, or oral administration of the CMV vector.

53. The method, CMV vector, pharmaceutical composition, or immunogenic composition for use, or use in manufacture of any of claims 30-42, 45-46, and 49-52, further comprising identifying a T cell receptor (TCR) from the CD8+ and/or CD4+ T cells elicited by the CMV vector, wherein the TCR recognizes a MHC-E/influenza antigen-derived peptide complex.

54. The method, CMV vector, pharmaceutical composition, or immunogenic composition for use, or use in manufacture of any of claims 30-42 and 47-52, further comprising identifying a TCR from the CD8+ and/or CD4+ T cells elicited by the CMV vector, wherein the TCR recognizes a MHC-II/influenza antigen-derived peptide complex.

55. The method, CMV vector, pharmaceutical composition, or immunogenic composition for use, or use in manufacture of any of claims 30-44 and 49-52, further comprising identifying a TCR from the CD8+ and/or CD4+ T cells elicited by the CMV vector, wherein the TCR recognizes a MHC-I/influenza antigen-derived peptide complex.

56. The method, CMV vector, pharmaceutical composition, or immunogenic composition for use, or use in manufacture of any of claims 53-55, wherein the TCR is identified by DNA or RNA sequencing.

57. A method of generating CD8+ and/or CD4+ T cells that recognize MHC-E-peptide complexes, the method comprising:
(1) administering to a subject the CMV vector of any of claims 3-27 in an amount effective to generate a set of CD8+ and/or CD4+ T cells that recognize MHC-E/peptide complexes;
(2) identifying a first TCR from the set of CD8+ and/or CD4+ T cells, wherein the first TCR recognizes a MHC-E/influenza antigen-derived peptide complex;
(3) isolating one or more CD8+ and/or CD4+ T cells from the subject; and
(4) transfecting the one or more CD8+ and/or CD4+ T cells with an expression vector, wherein the expression vector comprises a nucleic acid sequence encoding a second TCR and a promoter operably linked to the nucleic acid sequence encoding the second TCR, wherein the second TCR comprises CDR3α and CDR3β of the first TCR, thereby generating one or more transfected CD8+ and/or CD4+ T cells that recognize a MHC-E/influenza antigen-derived peptide complex.

58. The method of claim 57, wherein the first TCR is identified by DNA or RNA sequencing.

59. The method of any of claims 57-58, wherein the second TCR comprises CDR1α, CDR2α, CDR3α, CDR1β, CDR2β, and CDR3β of the first TCR.

60. The method of any of claims 57-59, wherein the nucleic acid sequence encoding the second TCR is identical to the nucleic acid sequence encoding the first TCR.

61. The method of any of claims 57-60, wherein administering the CMV vector to the subject comprises intravenous, intramuscular, intraperitoneal, or oral administration of the CMV vector to the subject.

62. The method of any of claims 57-61, wherein the subject is a human or nonhuman primate.

63. The method of any of claims 57-62, further comprising administering the transfected CD8+ and/or CD4+ T cells to the subject to treat a pathogenic infection.

64. The method of claim 63, wherein the pathogen is an influenza virus.

65. The method of claim 64, wherein the influenza virus is a H1N1 virus, a H5N1 virus, a H2N2 virus, or a H3N2 virus.

66. The method of claim 63, wherein the pathogen is heterologous to the antigens encoded on the CMV vector.

67. The method of claim 66, wherein the pathogen is a H5N1 virus, a H2N2 virus, or a H3N2 virus.

68. A method of generating CD8+ and/or CD4+ T cells that recognize MHC-E-peptide complexes, the method comprising:
(1) administering to a first subject the CMV vector of claims 3-27 in an amount effective to generate a set of CD8+ and/or CD4+ T cells that recognize MHC-E/peptide complexes;
(2) identifying a first TCR from the set of CD8+ T cells and/or CD4+ T cells, wherein the first TCR recognizes a MHC-E/influenza antigen-derived peptide complex;
(3) isolating one or more CD8+ and/or CD4+ T cells from a second subject; and
(4) transfecting the one or more CD8+ and/or CD4+ T cells with an expression vector, wherein the expression vector comprises a nucleic acid sequence encoding a second TCR and a promoter operably linked to the nucleic acid sequence encoding the second TCR, wherein the second TCR comprises CDR3α and CDR3β of the first TCR, thereby generating one or more transfected CD8+ and/or CD4+ T cells that recognize a MHC-E/influenza antigen-derived peptide complex.

69. The method of claim 68, wherein the first TCR is identified by DNA or RNA sequencing.

70. The method of any of claims 68-69, wherein the first subject is a human or nonhuman primate.

71. The method of any of claims 68-70, wherein the second subject is a human or nonhuman primate.

72. The method of any of claims 68-71, wherein the first subject is a non-human primate and the second subject is a human and wherein the second TCR is a chimeric non-human primate-human TCR comprising the non-human primate CDR3α and CDR3β of the first TCR.

73. The method of any of claims 68-71, wherein the second TCR comprises the non-human primate CDR1α, CDR2α, CDR3α, CDR1β, CDR2β, and CDR3β of the first TCR.

74. The method of any of claims 68-71, wherein the nucleic acid sequence encoding the second TCR is identical to the nucleic acid sequence encoding the first TCR.

75. The method of any of claims 68-71, wherein the second TCR is a chimeric TCR.

76. The method of any of claims 68-71, wherein the nucleic acid sequence encoding the second TCR is identical to the nucleic acid sequence encoding the first TCR.

77. The method of any of claims 68-76, wherein administering the CMV vector to the subject comprises intravenous, intramuscular, intraperitoneal, or oral administration of the CMV vector to the subject.

78. The method of any of claims 68-77, further comprising administering the transfected CD8+ and/or CD4+ T cells to the subject to treat a pathogenic infection.

79. The method of any of claims 68-78, wherein the pathogen is an influenza virus.

80. The method of claim 79, wherein the influenza virus is a H1N1 virus, a H5N1 virus, a H2N2 virus, or a H3N2 virus.

81. The method of claim 78, wherein the pathogen is heterologous to the antigens encoded on the CMV vector.

82. The method of claim 81, wherein the pathogen is a H5N1 virus, a H2N2 virus, or a H3N2 virus.

83. A method of generating CD8+ and/or CD4+ T cells that recognize MHC-II-peptide complexes, the method comprising:
(1) administering to a subject the CMV vector of claims 3-27 in an amount effective to generate a set of CD8+ and/or CD4+ T cells that recognize MHC-II/peptide complexes;
(2) identifying a first TCR from the set of CD8+ and/or CD4+ T cells, wherein the first TCR recognizes a MHC-II/influenza antigen-derived peptide complex;
(3) isolating one or more CD8+ and/or CD4+ T cells from the subject; and
(4) transfecting the one or more CD8+ and/or CD4+ T cells with an expression vector, wherein the expression vector comprises a nucleic acid sequence encoding a second TCR and a promoter operably linked to the nucleic acid sequence encoding the second TCR, wherein the second TCR comprises CDR3α and CDR3β of the first TCR, thereby generating one or more transfected CD8+ and/or CD4+ T cells that recognize a MHC-II/influenza antigen-derived peptide complex.

84. The method of claim 83, wherein the first TCR is identified by DNA or RNA sequencing.

85. The method of any of claims 83-84, wherein the second TCR comprises CDR1α, CDR2α, CDR3α, CDR1β, CDR2β, and CDR3β of the first TCR.

86. The method of any of claims 83-85, wherein the nucleic acid sequence encoding the second TCR is identical to the nucleic acid sequence encoding the first TCR.

87. The method of any of claims 83-86, wherein administering the CMV vector to the subject comprises intravenous, intramuscular, intraperitoneal, or oral administration of the CMV vector to the subject.

88. The method of any of claims 83-87, wherein the subject is a human or nonhuman primate.

89. The method of any of claims 83-88, further comprising administering the transfected CD8+ and/or CD4+ T cells to the subject to treat a pathogenic infection.

90. The method of any of claims 83-89, wherein the pathogen is an influenza virus.

91. The method of claim 90, wherein the influenza virus is a H1N1 virus, a H5N1 virus, a H2N2 virus, or a H3N2 virus.

92. The method of claim 89, wherein the pathogen is heterologous to the antigens encoded on the CMV vector.

93. The method of claim 92, wherein the pathogen is a H5N1 virus, a H2N2 virus, or a H3N2 virus.

94. A method of generating CD8+ and/or CD4+ T cells that recognize MHC-II-peptide complexes, the method comprising:
(1) administering to a first subject the CMV vector of claims 3-27 in an amount effective to generate a set of CD8+ and/or CD4+ T cells that recognize MHC-II/peptide complexes;
(2) identifying a first TCR from the set of CD8+ and/or CD4+ T cells, wherein the first TCR recognizes a MHC-II/influenza antigen-derived peptide complex;
(3) isolating one or more CD8+ and/or CD4+ T cells from a second subject; and
(4) transfecting the one or more CD8+ and/or CD4+ T cells with an expression vector, wherein the expression vector comprises a nucleic acid sequence encoding a second TCR and a promoter operably linked to the nucleic acid sequence encoding the second TCR, wherein the second TCR comprises CDR3α and CDR3β of the first TCR, thereby generating one or more transfected CD8+ and/or CD4+ T cells that recognize a MHC-II/influenza antigen-derived peptide complex.

95. The method of claim 94, wherein the first TCR is identified by DNA or RNA sequencing.

96. The method of any of claims 94-95, wherein the first subject is a human or nonhuman primate.

97. The method of any of claims 94-96, wherein the second subject is a human or nonhuman primate.

98. The method of any of claims 94-97, wherein the first subject is a non-human primate and the second subject is a human and wherein the second TCR is a chimeric non-human primate-human TCR comprising the non-human primate CDR3α and CDR3β of the first TCR.

99. The method of any of claims 94-97, wherein the second TCR comprises the non-human primate CDR1α, CDR2α, CDR3α, CDR1β, CDR2β, and CDR3β of the first TCR.

100. The method of any of claims 94-97, wherein the nucleic acid sequence encoding the second TCR is identical to the nucleic acid sequence encoding the first TCR.

101. The method of any of claims 94-97, wherein the second TCR is a chimeric TCR.

102. The method of any of claims 94-97, wherein the nucleic acid sequence encoding the second TCR is identical to the nucleic acid sequence encoding the first TCR.

103. The method of any of claims 94-102, wherein administering the CMV vector to the subject comprises intravenous, intramuscular, intraperitoneal, or oral administration of the CMV vector to the subject.

104. The method of any of claims 94-103, further comprising administering the transfected CD8+ and/or CD4+ T cells to the subject to treat a pathogenic infection.

105. The method of claim 104, wherein the pathogen is an influenza virus.

106. The method of claim 105, wherein the influenza virus a H1N1 virus, a H5N1 virus, a H2N2 virus, or a H3N2 virus.

107. The method of claim 104, wherein the pathogen is heterologous to the antigens encoded on the CMV vector.

108. The method of claim 107, wherein the pathogen a H5N1 virus, a H2N2 virus, or a H3N2 virus.

109. A method of generating CD8+ and/or CD4+ T cells that recognize MHC-I-peptide complexes, the method comprising:
(1) administering to a subject the CMV vector of claims 1-2 and 4-27 in an amount effective to generate a set of CD8+ and/or CD4+ T cells that recognize MHC-I/peptide complexes;
(2) identifying a first TCR from the set of CD8+ and/or CD4+ T cells, wherein the first TCR recognizes a MHC-I/influenza antigen-derived peptide complex;
(3) isolating one or more CD8+ and/or CD4+ T cells from the subject; and
(4) transfecting the one or more CD8+ and/or CD4+ cells with an expression vector, wherein the expression vector comprises a nucleic acid sequence encoding a second TCR and a promoter operably linked to the nucleic acid sequence encoding the second TCR, wherein the second TCR comprises CDR3α and CDR3β of the first TCR, thereby generating one or more transfected CD8+ and/or CD4+ T cells that recognize a MHC-I/influenza antigen-derived peptide complex.

110. The method of claim 109, wherein the first TCR is identified by DNA or RNA sequencing.

111. The method of any of claims 109-110, wherein the second TCR comprises CDR1α, CDR2α, CDR3α, CDR1β, CDR2β, and CDR3β of the first TCR.

112. The method of any of claims 109-111, wherein the nucleic acid sequence encoding the second TCR is identical to the nucleic acid sequence encoding the first TCR.

113. The method of any of claims 109-112, wherein administering the CMV vector to the subject comprises intravenous, intramuscular, intraperitoneal, or oral administration of the CMV vector to the subject.

114. The method of any of claims 109-113, wherein the subject is a human or nonhuman primate.

115. The method of any of claims 109-114, further comprising administering the transfected CD8+ and/or CD4+ T cells to the subject to treat a pathogenic infection.

116. The method of claim 115, wherein the pathogen is an influenza virus.

117. The method of claim 116, wherein the influenza virus is a H1N1 virus, a H5N1 virus, a H2N2 virus, or a H3N2 virus.

118. The method of claim 115, wherein the pathogen is heterologous to the antigens encoded on the CMV vector.

119. The method of claim 118, wherein the pathogen is a H5N1 virus, a H2N2 virus, or a H3N2 virus.

120. A method of generating CD8+ and/or CD4+ T cells that recognize MHC-I-peptide complexes, the method comprising:
(1) administering to a first subject the CMV vector of claims 1-2 and 4-27 in an amount effective to generate a set of CD8+ and/or CD4+ T cells that recognize MHC-I/peptide complexes;
(2) identifying a first TCR from the set of CD8+ and/or CD4+ T cells, wherein the first TCR recognizes a MHC-I/influenza antigen-derived peptide complex;
(3) isolating one or more CD8+ and/or CD4+ T cells from a second subject; and
(4) transfecting the one or more CD8+ and/or CD4+ T cells with an expression vector, wherein the expression vector comprises a nucleic acid sequence encoding a second TCR and a promoter operably linked to the nucleic acid sequence encoding the second TCR, wherein the second TCR comprises CDR3α and CDR3β of the first TCR, thereby generating one or more transfected CD8+ and/or CD4+ T cells that recognize a MHC-I/influenza antigen-derived peptide complex.

121. The method of claim 120, wherein the first TCR is identified by DNA or RNA sequencing.

122. The method of any of claims 120-121, wherein the first subject is a human or nonhuman primate.

123. The method of any of claims 120-122, wherein the second subject is a human or nonhuman primate.

124. The method of any of claims 120-123, wherein the first subject is a non-human primate and the second subject is a human and wherein the second TCR is a chimeric non-human primate-human TCR comprising the non-human primate CDR3α and CDR3β of the first TCR.

125. The method of any of claims 120-123, wherein the second TCR comprises the non-human primate CDR1α, CDR2α, CDR3α, CDR1β, CDR2β, and CDR3β of the first TCR.

126. The method of any of claims 120-123, wherein the nucleic acid sequence encoding the second TCR is identical to the nucleic acid sequence encoding the first TCR.

127. The method of any of claims 120-123, wherein the second TCR is a chimeric TCR.

128. The method of any of claims 120-123, wherein the nucleic acid sequence encoding the second TCR is identical to the nucleic acid sequence encoding the first TCR.

129. The method of any of claims 120-128, wherein administering the CMV vector to the subject comprises intravenous, intramuscular, intraperitoneal, or oral administration of the CMV vector to the subject.

130. The method of any of claims 120-129, further comprising administering the transfected CD8+ and/or CD4+ T cells to the subject to treat a pathogenic infection.

131. The method of claim 130, wherein the pathogen is an influenza virus.

132. The method of claim 131, wherein the influenza virus is a H1N1 virus, a H5N1 virus, a H2N2 virus, or a H3N2 virus.

133. The method of claim 130, wherein the pathogen is heterologous to the antigens encoded on the CMV vector.

134. The method of claim 133, wherein the pathogen is a H5N1 virus, a H2N2 virus, or a H3N2 virus.

135. A CD8+ and/or CD4+ T cell generated by the method of claims 57-134.

136. A method of treating or preventing a pathogenic infection in a subject, the method comprising administering the CD8+ and/or CD4+ T cell of claim 135.

137. Use of the CD8+ and/or CD4+ T cell of claim 135 in the manufacture of a medicament for use in treating or preventing a pathogenic infection in a subject.

138. The CD8+ and/or CD4+ T cell of claim 135 for use in treating or preventing a pathogenic infection in a subject.

139. The method, CMV vector for use, or use in manufacture of any of claims 136-138, wherein the pathogen is an influenza virus.

140. The method, CMV vector for use, or use in manufacture of claim 139, wherein the influenza virus is a H1N1 virus, a H5N1 virus, a H2N2 virus, or a H3N2 virus.

141. The method, CMV vector for use, or use in manufacture of any of claims 136-138, wherein the pathogen is heterologous to the antigens encoded on the CMV vector.

142. The method, CMV vector for use, or use in manufacture of claim 141, wherein the pathogen is a H5N1 virus, a H2N2 virus, or a H3N2 virus.

143. A recombinant cynomolgus cytomegalovirus (CyCMV) vector comprising at least one heterologous antigen, wherein the vector does not express an active UL128 protein or ortholog thereof; does not express an active UL130 protein or ortholog thereof; and does not express an active UL146 protein or ortholog thereof.

144. The CyCMV vector of claim 143, wherein the CyCMV vector does not express an active pp71 protein or ortholog thereof.

145. The CyCMV vector of any of claims 143-144, wherein the at least one heterologous antigen comprises a pathogen-specific antigen, a tumor antigen, a tissue-specific antigen, or a host self-antigen.

146. The CyCMV vector of claim 145, wherein the pathogen specific antigen is derived from a pathogen selected from the group consisting of human immunodeficiency virus, simian immunodeficiency virus, herpes simplex virus type 1, herpes simplex virus type 2, hepatitis B virus, hepatitis C virus, papillomavirus, Plasmodium parasites, and Mycobacterium tuberculosis.

147. The CyCMV vector claim 145, wherein the tumor antigen is related to a cancer selected from the group consisting of acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, breast cancer, lung cancer, ovarian cancer, prostate cancer, pancreatic cancer, colon cancer, renal cell carcinoma (RCC), and germ cell tumors.

148. A pharmaceutical composition comprising the CyCMV vector of any of claims 143-147 and a pharmaceutically acceptable carrier.

149. An immunogenic composition comprising the CyCMV vector of any of claims 143-147 and a pharmaceutically acceptable carrier.

150. A method of generating an immune response in a subject, comprising administering to the subject the CyCMV vector of any of claims 143-147 in an amount effective to elicit a CD8+ and/or CD4+ T cell response.

151. Use of the CyCMV vector of any of claims 143-147 in the manufacture of a medicament for use in generating an immune response in a subject.

152. The CyCMV vector of any of claims 143-147 for use in generating an immune response in a subject.

153. A method of generating an immune response in a subject to a pathogenic infection, comprising administering to the subject the CyCMV vector of any of claims 143-147 in an amount effective to elicit a CD8+ and/or CD4+ T cell response to a pathogen.

154. Use of the CyCMV vector of any of claims 143-147 in the manufacture of a medicament for use in generating an immune response to a pathogen in a subject.

155. The CyCMV vector of any of claims 143-147 for use in generating an immune response to a pathogen in a subject.

156. A method of treating or preventing a pathogenic infection in a subject, comprising administering the CyCMV vector of any of claims 143-147 in an amount effective to elicit a CD8+ and/or CD4+ T cell response to the pathogen.

157. Use of the CyCMV vector of any of claims 143-147 in the manufacture of a medicament for use in treating or preventing a pathogenic infection in a subject.

158. The CyCMV vector of any of claims 143-147 for use in treating or preventing a pathogenic infection in a subject.

159. The CyCMV vector of any of claims 153-158, wherein the pathogen is a pathogen selected from the group consisting of: human immunodeficiency virus, simian immunodeficiency virus, herpes simplex virus type 1, herpes simplex virus type 2, hepatitis B virus, hepatitis C virus, papillomavirus, Plasmodium parasites, and Mycobacterium tuberculosis.

160. A method of treating a tumor in a subject, comprising administering the CyCMV vector of any of claims 143-147 in an amount effective to elicit a CD8+ and/or CD4+ T cell response to the tumor.

161. Use of the CyCMV vector of any of claims 143-147 in the manufacture of a medicament for use in treating a tumor in a subject.

162. The CyCMV vector of any of claims 143-147 for use in treating a tumor in a subject.

163. The CyCMV vector of any of claims 160-162, wherein the tumor is related to a cancer selected from the group consisting of: acute myelogenous leukemia, chronic myelogenous leukemia, myelodysplastic syndrome, acute lymphoblastic leukemia, chronic lymphoblastic leukemia, non-Hodgkin's lymphoma, multiple myeloma, malignant melanoma, breast cancer, lung cancer, ovarian cancer, prostate cancer, pancreatic cancer, colon cancer, renal cell carcinoma (RCC), and germ cell tumors.

164. The method, CMV vector, pharmaceutical composition, or immunogenic composition for use, or use in manufacture of any of claims 143-163, wherein at least 10% of the CD8+ and/or CD4+ T cells elicited by the recombinant CMV vector are restricted by MHC-E or an ortholog thereof.

165. The method, CMV vector, pharmaceutical composition, or immunogenic composition for use, or use in manufacture of claim 164, wherein at least 20%, at least 30%, at least 40%, at least 50%, at least 60%, at least 70%, at least 75%, at least 80%, at least 85%, at least 90%, or at least 95% of the CD8+ and/or CD4+ T cells elicited by the recombinant CMV vector are restricted by MHC-E or an ortholog thereof.

166. The method, CMV vector, pharmaceutical composition, or immunogenic composition for use, or use in manufacture of any of claims 143-163, wherein at least 10% of the CD8+ and/or CD4+ T cells elicited by the recombinant CMV vector are restricted by MHC-II or an ortholog thereof.

167. The method, CMV vector, pharmaceutical composition, or immunogenic composition for use, or use in manufacture of claim 166, wherein at least 20%, at least 30%, at least 40%, at least 50%, at least 60%, at least 70%, at least 75%, at least 80%, at least 85%, at least 90%, or at least 95% of the CD8+ and/or CD4+ T cells elicited by the recombinant CMV vector are restricted by MHC-II or an ortholog thereof.

168. The method, CMV vector, pharmaceutical composition, or immunogenic composition for use, or use in manufacture of claims 143-163, wherein fewer than 10%, fewer than 20%, fewer than 30%, fewer than 40%, or fewer than 50% of the CD8+ and/or CD4+ T cells elicited by the recombinant CMV vector are restricted by MHC-I or an ortholog thereof.

169. The method, CMV vector, pharmaceutical composition, or immunogenic composition for use, or use in manufacture of claim 168, wherein fewer than 10% of the CD8+ and/or CD4+ T cells elicited by the recombinant CMV vector are restricted by MHC-I or an ortholog thereof.

170. The method, CMV vector, pharmaceutical composition, or immunogenic composition for use, or use in manufacture of any of claims 150-169, wherein the subject is a human or non-human primate.

171. The method, CMV vector, pharmaceutical composition, or immunogenic composition for use, or use in manufacture of any of claims 150-170, wherein administering the CMV vector comprises subcutaneous, intravenous, intramuscular, intraperitoneal, or oral administration of the CMV vector.

172. The method, CMV vector, pharmaceutical composition, or immunogenic composition for use, or use in manufacture of any of claims 150-171, further comprising identifying a TCR from the CD8+ and/or CD4+ T cells elicited by the CMV vector, wherein the TCR recognizes a MHC-E/heterologous antigen-derived peptide complex.

173. The method, CMV vector, pharmaceutical composition, or immunogenic composition for use, or use in manufacture of any of claims 150-171, further comprising identifying a TCR from the CD8+ and/or CD4+ T cells elicited by the CMV vector, wherein the TCR recognizes a MHC-II/heterologous antigen-derived peptide complex.

174. The method, CMV vector, pharmaceutical composition, or immunogenic composition for use, or use in manufacture of any of claims 172-173, wherein the TCR is identified by DNA or RNA sequencing.
